(19) United States
(12) Patent Application Publication
(10) Pub. No.: US 2013/0203053 A1
(43) Pub. Date: Aug. 8, 2013

(54) METHODS FOR IMPROVING INFLAMMATORY BOWEL DISEASE DIAGNOSIS

(71) Applicant: Nestec S.A., Vevey (CH)

(72) Inventors: Fred Princen, La Jolla, CA (US); Sharat Singh, Rancho Santa Fe, CA (US)

(73) Assignee: NESTEC S.A., Vevey (CH)

(21) Appl. No.: 13/686,861

(22) Filed: Nov. 27, 2012

Related U.S. Application Data
(63) Continuation of application No. PCT/US2011/039174, filed on Jun. 3, 2011.
(60) Provisional application No. 61/393,588, filed on Oct. 15, 2010.
(60) Provisional application No. 61/354,141, filed on Jun. 11, 2010.
(60) Provisional application No. 61/351,837, filed on Jun. 4, 2010.

(51) Int. Cl. C12Q 1/68

(52) U.S. Cl. CPC C12Q 1/6883
USPC 435/6.11

(57) ABSTRACT

The present invention provides methods and systems to diagnose the ulcerative colitis (UC) subtype of inflammatory bowel disease (IBD) by detecting the presence or absence of one or more variant alleles in the GLI1, MDR1, and/or ATG16L1 genes. Advantageously, with the present invention, it is possible to provide a diagnosis of UC and to differentiate between UC and Crohn's disease (CD) with increased accuracy.

### CROSS REFERENCE TO RELATED APPLICATIONS

This application is a continuation of PCT/US2011/039174, filed Jun. 3, 2011, which application claims priority to U.S. Provisional Application No. 61/351,837, filed Jun. 4, 2010, U.S. Provisional Application No. 61/354,141, filed Jun. 11, 2010, and U.S. Provisional Application No. 61/393,588, filed Oct. 15, 2010, the disclosures of which are hereby incorporated by reference in their entirety for all purposes.

### BACKGROUND OF THE INVENTION

Inflammatory bowel disease (IBD), which occurs world-wide and afflicts millions of people, is the collective term used to describe three gastrointestinal disorders of unknown etiology: Crohn's disease (CD), ulcerative colitis (UC), and indeterminate colitis (IC). IBD, together with irritable bowel syndrome (IBS), will affect one-half of all Americans during their lifetime, at a cost of greater than $2.6 billion dollars for IBD and greater than $8 billion dollars for IBS. A primary determinant of these high medical costs is the difficulty of diagnosing digestive diseases and how these diseases will progress. The cost of IBD and IBS is compounded by lost productivity, with people suffering from these disorders missing at least 8 more days of work annually than the national average.

Inflammatory bowel disease has many symptoms in common with irritable bowel syndrome, including abdominal pain, chronic diarrhea, weight loss, and cramping, making definitive diagnosis extremely difficult. Of the 5 million people suspected of suffering from IBD in the United States, only 1 million are diagnosed as having IBD. The difficulty in differentially diagnosing IBD and determining its outcome hampers early and effective treatment of these diseases. Thus, there is a need for rapid and sensitive testing methods for prognosticating the severity of IBD.

Although some progress has been made in diagnosing clinical subtypes of IBD, there remains a need for methods for use in differentiating between Crohn's disease (CD) and ulcerative colitis (UC). A such, there is a need for improved methods for diagnosing UC as well as differentiating between CD and UC in an individual who has been diagnosed with IBD. Since 70% of CD patients will ultimately need a GI surgical operation, the ability to differentiate between those patients who will need surgery in the future is important. The present invention satisfies these needs and provides related advantages as well.

### BRIEF SUMMARY OF THE INVENTION

In certain aspects, the present invention provides methods and systems to diagnose the ulcerative colitis (UC) subtype of inflammatory bowel disease (IBD). Advantageously, with the present invention, it is possible to aid in, assist in, and/or facilitate diagnosing UC and differentiating between UC and CD with improved clinical parameters such as sensitivity, specificity, negative predictive value, positive predictive value, overall accuracy, and combinations thereof.

In particular embodiments, the present invention provides methods and systems to diagnose UC and/or to differentiate between clinical subtypes of IBD such as UC and CD by analyzing a sample to determine the presence or absence of one, two, three, four, or more variant alleles (e.g., single nucleotide polymorphisms or SNPs) in the GLI1 (e.g., rs2228224 and/or rs2228226), MDR1 (e.g., rs2032582), and/or ATG16L1 (e.g., rs2241880) genes. In certain aspects of these embodiments, the present invention may further include analyzing a sample to determine the presence (or absence) or concentration level of one or more serological markers such as, e.g., ANCA (e.g., by ELISA) and/or pANCA (e.g., by an indirect fluorescent antibody (IFA) assay), to further improve the diagnosis of UC (e.g., by increasing the sensitivity of UC diagnosis) and/or to further improve distinguishing UC from other IBD subtypes such as CD or IC.

In certain embodiments, the present invention provides assay methods which are performed in vitro by analyzing a sample obtained from an individual (e.g., an individual previously diagnosed with IBD) for the presence or absence of one, two, three, four, or more variant alleles (e.g., SNPs) in the GLI1 (e.g., rs2228224 and/or rs2228226), MDR1 (e.g., rs2032582), and/or ATG16L1 (e.g., rs2241880) genes. In preferred embodiments, the assay methods of the invention aid in, assist in, and/or facilitate diagnosing UC and differentiating between UC and CD.

Other objects, features, and advantages of the present invention will be apparent to one of skill in the art from the following detailed description and figures.

### BRIEF DESCRIPTION OF THE DRAWINGS

FIG. 1 shows that the accuracy of the predictions was assessed using a Receiver Operator Characteristic (ROC) curve. In particular, the ROC curve was employed to predict the accuracy of the serological and genetic marker association with UC tests. Under this assessment, the performance of the test is indicated via the AUC (Area Under the Curve) statistic with confidence intervals. For ANCA/pANCA, the area under the ROC curve (AUC) was 0.793 (95% CI: 0.726-0.861). For ANCA/pANCA and the three genetic variants, the AUC was 0.856 (95% CI: 0.799-0.912), thus confirming the increased accuracy of the model in discriminating healthy control from UC when adding the three genetic variants to ANCA/pANCA.

FIG. 2 shows that the accuracy of the predictions was assessed using a ROC curve. In particular, the ROC curve was employed to predict the accuracy of the serological and genetic marker association with UC tests. Under this assessment, the performance of the test is indicated via the AUC statistic with confidence intervals. For ANCA/pANCA, the area under the ROC curve (AUC) was 0.793 (95% CI: 0.726-0.861). For ANCA/pANCA and the two genetic variants, the AUC was 0.853 (95% CI: 0.801-0.905), thus confirming the increased accuracy of the model in discriminating healthy control from UC when adding the two genetic variants to ANCA/pANCA.

FIG. 3 shows the pANCA staining pattern by immunofluorescence followed by DNAse treatment on fixed neutrophils.

FIG. 4 shows the use of ROC analysis to compare the diagnostic accuracy of ANCA/pANCA alone to the two gene variants, GLI1 (G933D) and MDR1 (A893S), when combined with ANCA/pANCA. The addition of the two gene variants to ANCA/pANCA increased the area under the curve from 0.802 (95% CI: 0.737-0.868) to 0.853 (95% CI: 0.801-0.905).

### DETAILED DESCRIPTION OF THE INVENTION

### I. Introduction

The present invention is based, in part, upon the surprising discovery that the accuracy of diagnosing UC or differentiating between UC and CD can be substantially improved by determining the genotype of certain markers in a biological sample from an individual. As such, in one embodiment, the present invention provides diagnostic platforms based on a genetic panel of markers.

In certain aspects, the present invention provides methods and systems to diagnose UC and to differentiate between UC and other clinical subtypes of IBD such as CD or IC. In particular embodiments, the methods and systems of the present invention utilize one or a plurality of (e.g., multiple) genetic markers, alone or in combination with one or a plurality of (e.g., multiple) serological and/or protein markers, and alone or in combination with one or a plurality of (e.g., multiple) algorithms or other types of statistical analysis (e.g., quartile analysis), to aid or assist in identifying patients with UC and providing physicians with valuable diagnostic insight. In other embodiments, the methods and systems of the present invention find utility in guiding therapeutic decisions of patients with advanced disease.

In certain instances, the methods and systems of the present invention comprise a step having a “transformation” or “machine” associated therewith. For example, an ELISA technique may be performed to measure the presence or concentration level of many of the markers described herein. An ELISA includes transformation of the marker, e.g., an auto-antibody, into a complex between the marker (e.g., auto-antibody) and a binding agent (e.g., antigen), which then can be measured with a labeled secondary antibody. In many instances, the label is an enzyme which transforms a substrate into a detectable product. The detectable product measurement can be performed using a plate reader such as a spectrophotometer. In other instances, genetic markers are determined using various amplification techniques such as PCR. Method steps including amplification such as PCR result in the transformation of single or double strands of nucleic acid into multiple strands for detection. The detection can include the use of a fluorophore, which is performed using a machine such as a fluorometer.

### II. Definitions

As used herein, the following terms have the meanings ascribed to them unless specified otherwise.

The term “classifying” includes “associating” or “categorizing” a sample or an individual with a disease state or prognosis. In certain instances, “classifying” is based on statistical evidence, empirical evidence, or both. In certain embodiments, the methods and systems of classifying use a so-called training set of samples from individuals with known disease states or prognoses. Once established, the training data set serves as a basis, model, or template against which the features of an unknown sample from an individual are compared, in order to classify the unknown disease state or provide a prognosis of the disease state in the individual. In some instances, “classifying” is akin to diagnosing the disease state and/or differentiating the disease state from another disease state. In other instances, “classifying” is akin to providing a prognosis of the disease state in an individual diagnosed with the disease state.

The term “inflammatory bowel disease” or “IBD” includes gastrointestinal disorders such as, e.g., Crohn's disease (CD), ulcerative colitis (UC), and indeterminate colitis (IC). Inflammatory bowel diseases (e.g., CD, UC, and IC) are distinguished from all other disorders, syndromes, and abnormalities of the gastroenterological tract, including irritable bowel syndrome (IBS). U.S. Patent Publication No. 20080131439, entitled “Methods of Diagnosing Inflammatory Bowel Disease” and U.S. Patent Publication No. 20100099083 are both incorporated herein by reference in their entirety for all purposes.

The term “biological sample,” “sample” and variants thereof is used herein to include a biological specimen obtained or isolated from an individual. Suitable samples include for example but are not limited to blood, whole blood, portions of blood, tissue, saliva, cheek cells, hair, bodily fluids, urine, plasma, serum, cerebrospinal fluid, buccal swabs, mucus, urine, stools, spermatozoids, vaginal secretions, lymph, amniotic fluid, pleural liquid, tears, any other bodily fluid, tissue samples (e.g., biopsy), and cellular extracts thereof (e.g., red blood cellular extract). In a preferred embodiment, the sample is a serum sample. The use of samples such as serum, saliva, and urine is well known in the art (see, e.g., Hashida et al., J. Clin. Lab. Anal., 11:267-86 (1997)). One skilled in the art will appreciate that samples such as serum samples can be diluted prior to the analysis of marker levels.

The term “marker” includes any biochemical marker, serological marker, genetic marker, or other clinical or echographic characteristic that can be used in aiding, assisting, and/or improving the diagnosis of IBD, CD, or UC, in the prediction of the probable course and outcome of IBD, CD, or UC, and/or in the prediction of the likelihood of recovery from the disease. Non-limiting examples of such markers include genetic markers such as variant alleles in the GLI1 (e.g., rs2228224 and/or rs2228226), MDR1 (e.g., rs2032582), ATG16L1 (e.g., rs2241880), and/or NOD2/CARD15 genes; serological markers such as an anti-neutrophil antibody (e.g., ANCA, pANCA, and the like), an anti-Saccharomyces cerevisiae antibody (e.g., ASCA-IgA, ASCA-IgG), an antimicrobial antibody (e.g., anti-OmpC antibody, anti-I2 antibody, anti-flagellin antibody), an acute phase protein (e.g., CRP), an apolipoprotein (e.g., SAA), a defensin (e.g., β defensin), a growth factor (e.g., EGF), a cytokine (e.g., TWEAK, IL-1β, IL-6), a cadherin (e.g., E-cadherin), a cellular adhesion molecule (e.g., ICAM-1, VCAM-1); and combinations thereof. In some embodiments, the markers are utilized in combination with a statistical analysis to provide a diagnosis or prognosis of IBD, CD, or UC in an individual. In certain instances, the diagnosis can be IBD or a clinical subtype thereof such as CD, UC, or IC. In certain other instances, the prognosis can be the need for surgery (e.g., the likelihood or risk of needing small bowel surgery), development of a clinical subtype of CD or UC (e.g., the likelihood or risk of being susceptible to a particular clinical subtype CD or UC such as the stricturing, penetrating, or inflammatory CD subtype), development of one or more clinical factors (e.g., the likelihood or risk of being susceptible to a particular clinical factor), development of intestinal cancer (e.g., the likelihood or risk of being susceptible to intestinal cancer), or recovery from the disease (e.g., the likelihood of remission).

The present invention relies, in part, on determining the presence (or absence) or level (e.g., concentration) of at least one marker in a sample obtained from an individual. As used herein, the term “detecting the presence of at least one marker” includes determining the presence of each marker of interest by using any quantitative or qualitative assay known to one of skill in the art. In certain instances, qualitative assays that determine the presence or absence of a particular trait, variable, genotype, and/or biochemical or serological substance (e.g., protein or antibody) are suitable for detecting each marker of interest. In certain other instances, quantitative assays that determine the presence or absence of DNA, RNA, protein, antibody, or activity are suitable for detecting each marker of interest. As used herein, the term “detecting the level of at least one marker” includes determining the level of each marker of interest by using any direct or indirect quantitative assay known to one of skill in the art. In certain instances, quantitative assays that determine, for example, the relative or absolute amount of DNA, RNA, protein, antibody, or activity are suitable for detecting the level of each marker of interest. One skilled in the art will appreciate that any assay useful for detecting the level of a marker is also useful for detecting the presence or absence of the marker.

The term “individual,” “subject,” or “patient” typically includes humans, but also includes other animals such as, e.g., other primates, rodents, canines, felines, equines, ovines, porcines, and the like.

The term “clinical factor” includes a symptom in an individual that is associated with IBD, CD, or UC. Examples of clinical factors include, without limitation, diarrhea, abdominal pain, cramping, fever, anemia, weight loss, anxiety, depression, and combinations thereof. In some embodiments, a diagnosis or prognosis of IBD, CD, or UC is based upon a combination of analyzing a sample obtained from an individual to determine the presence, level, or genotype of one or more markers by applying one or more statistical analyses and determining whether the individual has one or more clinical factors.

The term “symptom” or “symptoms” and variants thereof includes any sensation, change or perceived change in bodily function that is experienced by an individual and is associated with a particular diseases or that accompanies a disease and is regarded as an indication of the disease. Disease for which symptoms in the context of the present invention can be associated with include inflammatory bowel disease (IBD), ulcerative colitis (UC) or Crohn's disease (CD).

In a preferred aspect, the methods of invention are used after an individual has been diagnosed with IBD. However, in other instances, the methods can be used to diagnose IBD or can be used as a “second opinion” if, for example, IBD is suspected or has been previously diagnosed using other methods. In preferred aspects, the methods can be used to diagnose UC or differentiate between UC and CD. The term “diagnosing IBD” and variants thereof includes the use of the methods and systems described herein to determine the presence or absence of IBD. The term “diagnosing UC” includes the use of the methods and systems described herein to determine the presence or absence of UC, as well as to differentiate between UC and CD. The terms can also include assessing the level of disease activity in an individual. In some embodiments, a statistical analysis is used to diagnose a mild, moderate, severe, or fulminant form of IBD or UC based upon the criteria developed by Truelove et al., Br. Med. J., 12:1041-1048 (1955). In other embodiments, a statistical analysis is used to diagnose a mild to moderate, moderate to severe, or severe to fulminant form of IBD or UC based upon the criteria developed by Hanauer et al., Am. J. Gastroenterol., 92:559-566 (1997). One skilled in the art will know of other methods for evaluating the severity of IBD or UC in an individual.

In certain instances, the methods of the invention are used in order to diagnose IBD, diagnose UC or differentiate between UC and CD. The methods can be used to monitor the disease, both progression and regression. The term “monitoring the progression or regression of IBD or UC” includes the use of the methods and marker profiles to determine the disease state (e.g., presence or severity of IBD or the presence of UC) of an individual. In certain instances, the results of a statistical analysis are compared to those results obtained for the same individual at an earlier time. In some aspects, the methods of the present invention can also be used to predict the progression of IBD or UC, e.g., by determining a likelihood for IBD or UC to progress either rapidly or slowly in an individual based on the presence or level of at least one marker in a sample. In other aspects, the methods of the present invention can also be used to predict the regression of IBD or UC, e.g., by determining a likelihood for IBD or UC to regress either rapidly or slowly in an individual based on the presence or level of at least one marker in a sample.

The term “gene” and variants thereof refers to the segment of DNA involved in producing a polypeptide chain; it includes regions preceding and following the coding region, such as the promoter and 3′-untranslated region, respectively, as well as intervening sequences (introns) between individual coding segments (exons).

The term “genotype” and variants thereof refers to the genetic composition of an organism, including, for example, whether a diploid organism is heterozygous or homozygous for one or more variant alleles of interest.

The terms “miRNA,” “microRNA” or “miR” and variants thereof are used interchangeably and include single-stranded RNA molecules of 21-23 nucleotides in length, which regulate gene expression. miRNAs are encoded by genes from whose DNA they are transcribed but miRNAs are not translated into protein (non-coding RNA); instead each primary transcript (a pri-miRNA) is processed into a short stem-loop structure called a pre-miRNA and finally into a functional miRNA. Mature miRs are partially complementary to one or more messenger RNA (mRNA) molecules, and their main function is to down-regulate gene expression. Embodiments described herein include both diagnostic and therapeutic applications.

The term “polymorphism” and variants thereof refers to the occurrence of two or more genetically determined alternative sequences or alleles in a population. A “polymorphic site” refers to the locus at which divergence occurs. Preferred polymorphic sites have at least two alleles, each occurring at a particular frequency in a population. A polymorphic locus may be as small as one base pair (e.g., single nucleotide polymorphism or SNP). Polymorphic markers include restriction fragment length polymorphisms, variable number of tandem repeats (VNTR's), hypervariable regions, minisatellites, dinucleotide repeats, trinucleotide repeats, tetranucleotide repeats, simple sequence repeats, and insertion elements such as Alu. The first identified allele is arbitrarily designated as the reference allele, and other alleles are designated as alternative alleles, “variant alleles,” or “variances.” The allele occurring most frequently in a selected population can sometimes be referred to as the “wild-type” allele. Diploid organisms may be homozygous or heterozygous for the variant alleles. The variant allele may or may not produce an observable physical or biochemical characteristic (“phenotype”) in an individual carrying the variant allele. For example, a variant allele may alter the enzymatic activity of a protein encoded by a gene of interest or in the alternative the variant allele may have no effect on the enzymatic activity of an encoded protein.

The term “single nucleotide polymorphism (SNP)” and variants thereof refers to a change of a single nucleotide with a polynucleotide, including within an allele. This can include the replacement of one nucleotide by another, as well as deletion or insertion of a single nucleotide. Most typically, SNPs are biallelic markers although tri- and tetra-allelic markers can also exist. By way of non-limiting example, a nucleic acid molecule comprising SNP A\C may include a C or A at the polymorphic position. For combinations of SNPs, the term “haplotype” is used, e.g. the genotype of the SNPs in a single DNA strand that are linked to one another. In some embodiments, the term “haplotype” can be used to describe a combination of SNP alleles, e.g., the alleles of the SNPs found together on a single DNA molecule. In further embodiments, the SNPs in a haplotype can be in linkage disequilibrium with one another.

The term “linkage disequilibrium” or “LD” and variants thereof refers to the situation wherein the alleles for two or more loci do not occur together in individuals sampled from a population at frequencies predicted by the product of their individual allele frequencies. In other words, markers that are in LD do not follow Mendel's second law of independent random segregation. Further, markers that are in high LD can be assumed to be located near each other and a marker or haplotype that is in high LD with a genetic trait can be assumed to be located near the gene that affects that trait. The physical proximity of markers can be measured in family studies where it is called linkage or in population studies where it is called linkage disequilibrium.

The term “skewed genotype distribution” and variants thereof refers to the situation where the genotype does not follow standard statistical parameters for being associated with a specific disease or control population; i.e., does not follow a standard, normal symmetric distribution pattern.

The term “specific” or “specificity” and variants thereof, when used in the context of polynucleotides capable of detecting variant alleles (e.g., polynucleotides that are capable of discriminating between different alleles), includes the ability to bind or hybridize or detect one variant allele without binding or hybridizing or detecting the other variant allele. In some embodiments, specificity can refer to the ability of a polynucleotide to detect the wild-type and not the mutant or variant allele. In other embodiments, specificity can refer to the ability of a polynucleotide to detect the mutant or variant allele and not the wild-type allele.

As used herein, the term “antibody” includes a population of immunoglobulin molecules, which can be polyclonal or monoclonal and of any isotype, or an immunologically active fragment of an immunoglobulin molecule. Such an immunologically active fragment contains the heavy and light chain variable regions, which make up the portion of the antibody molecule that specifically binds an antigen. For example, an immunologically active fragment of an immunoglobulin molecule known in the art as Fab, Fab′ or F(ab′)₂ is included within the meaning of the term antibody.

### III. Description of the Embodiments

The present invention provides methods and systems to diagnose ulcerative colitis (UC) and to differentiate between UC and Crohn's disease (CD). By identifying patients with complicated disease and assisting in assessing the specific disease type, the methods and systems described herein provide invaluable information to assess the severity of the disease and treatment options. In some embodiments, applying a statistical analysis to a profile of serological, protein, and/or genetic markers improves the accuracy of predicting IBD and UC, and also enables the selection of appropriate treatment options, including therapy such as biological, conventional, surgery, or some combination thereof.

In one aspect, the present invention provides a method for diagnosing ulcerative colitis (UC) in an individual diagnosed with inflammatory bowel disease (IBD) and/or suspected of having UC. In some embodiments, the method comprises: (i) analyzing a biological sample obtained from the individual to determine the presence or absence of a variant allele in a gene in a biological sample, wherein the gene is one or more of GLI1, MDR1, or ATG16L1; and (ii) associating the presence of the variant allele with a diagnosis of UC.

In some embodiments, the method of diagnosing UC employs detection of the GLI1 (rs2228224) variant allele. In other embodiments, the method of diagnosing UC employs detection of the GLI1 (rs2228226) variant allele. In some embodiments, the method of diagnosing UC employs detection of the MDR1 (rs2032582) variant allele. In further embodiments, the method of diagnosing UC employs detection of the ATG16L1 (rs2241880) variant allele.

In other embodiments, the method of diagnosing UC employs detection of one or more variant alleles selected from the group consisting of GLI1 (rs2228224), GLI1 (rs2228226), MDR1 (rs2032582), and ATG16L1 (rs2241880). In one particular embodiment, the method of diagnosing UC comprises detecting the GLI1 (rs2228224) and MDR1 (rs2032582) variant alleles. In another particular embodiment, the method of diagnosing UC comprises detecting the GLI1 (rs2228224) and ATG16L1 (rs2241880) variant alleles. In yet another particular embodiment, the method of diagnosing UC comprises detecting the MDR1 (rs2032582) and ATG16L1 (rs2241880) variant alleles. In still yet another particular embodiment, the method of diagnosing UC comprises detecting the GLI1 (rs2228224), MDR1 (rs2032582), and ATG16L1 (rs2241880) variant alleles.

In particular embodiments, the method described herein improves the diagnosis of UC compared to ANCA and/or pANCA-based methods of diagnosing UC.

In other embodiments, the method of diagnosing UC employs an additional step of analyzing the biological sample for the presence or level of a serological marker, wherein detection of the presence or level of the serological marker in conjunction with the presence of one or more variant alleles further improves the diagnosis of UC.

In yet other embodiments, the method of diagnosing UC employs detection of a serological marker selected from an anti-neutrophil antibody, an anti-Saccharomyces cerevisiae antibody, an antimicrobial antibody, an acute phase protein, an apolipoprotein, a defensin, a growth factor, a cytokine, a cadherin, or any combination of the markers described herein.

In further embodiments, the method of diagnosing UC utilizes an anti-neutrophil antibody that is selected from one of ANCA and pANCA, or a combination of ANCA and pANCA. In one embodiment, the anti-neutrophil antibody comprises an anti-neutrophil cytoplasmic antibody (ANCA) such as ANCA detected by an immunoassay (e.g., ELISA), a perinuclear anti-neutrophil cytoplasmic antibody (pANCA) such as pANCA detected by an immunohistochemical assay (e.g., IFA) or a DNAse-sensitive immunohistochemical assay, or a combination thereof.

In yet further additional embodiments, the method of diagnosing UC utilizes an anti-Saccharomyces cerevisiae antibody that is selected from the group consisting of anti-Saccharomyces cerevisiae immunoglobulin A (ASCA-IgA), anti-Saccharomyces cerevisiae immunoglobulin G (ASCA-IgG), and a combination thereof.

In yet other embodiments, the method of diagnosing UC utilizes an antimicrobial antibody that is selected from the group consisting of an anti-outer membrane protein C (anti-OmpC) antibody, an anti-I2 antibody, an anti-flagellin antibody, and a combination thereof.

In particular embodiments, the serological marker comprises or consists of ANCA, pANCA (e.g., pANCA IFA and/or DNAse-sensitive pANCA IFA), ASCA-IgA, ASCA-IgG, anti-OmpC antibody, anti-CBir-1 antibody, anti-I2 antibody, or a combination thereof.

In certain instances, the presence or absence of one, two, three, or more of the GLI1 (rs2228224), GLI1 (rs2228226), MDR1 (rs2032582), and/or ATG16L1 (rs2241880) SNPs is determined in combination with the presence (or absence) or (concentration) level of one, two, three, or more serological markers, e.g., ANCA (e.g., ANCA ELISA), pANCA (e.g., pANCA IFA and/or DNAse-sensitive pANCA IFA), ASCA-IgA, ASCA-IgG, anti-OmpC antibody, anti-CBir-1 antibody, anti-I2 antibody, or a combination thereof.

In one particular embodiment, the presence of the GLI1 (rs2228224), MDR1 (rs2032582), and ATG16L1 (rs2241880) SNPs in combination with the presence or level of ANCA (e.g., high ANCA levels by ELISA) and/or pANCA (e.g., pANCA-positive staining of alcohol-fixed neutrophils) can be employed to increase the sensitivity and/or accuracy of UC diagnosis. In another particular embodiment, the presence of the GLI1 (rs2228224) and MDR1 (rs2032582) SNPs in combination with the presence or level of ANCA (e.g., high ANCA levels by ELISA) and/or pANCA (e.g., pANCA-positive staining of alcohol-fixed neutrophils) can be employed to increase the sensitivity and/or accuracy of UC diagnosis.

The presence or absence of a variant allele in a genetic marker can be determined using an assay described in Section VI below. Assays that can be used to determine variant allele status include, but are not limited to, electrophoretic analysis assays, restriction length polymorphism analysis assays, sequence analysis assays, hybridization analysis assays, PCR analysis assays, allele-specific hybridization, oligonucleotide ligation allele-specific elongation/ligation, allele-specific amplification, single-base extension, molecular inversion probe, invasive cleavage, selective termination, restriction length polymorphism, sequencing, single strand conformation polymorphism (SSCP), single strand chain polymorphism, mismatch-cleaving, denaturing gradient gel electrophoresis, and combinations thereof. These assays have been well-described and standard methods are known in the art. See, e.g., Ausubel et al., Current Protocols in Molecular Biology, John Wiley & Sons, Inc. New York (1984-2008), Chapter 7 and Supplement 47; Theophilus et al., “PCR Mutation Detection Protocols,” Humana Press, (2002); Innis et al., PCR Protocols, San Diego, Academic Press, Inc. (1990); Maniatis, et al., Molecular Cloning: A Laboratory Manual, Cold Spring Harbor Lab., New York, (1982); Ausubel et al., Current Protocols in Genetics and Genomics, John Wiley & Sons, Inc. New York (1984-2008); and Ausubel et al., Current Protocols in Human Genetics, John Wiley & Sons, Inc. New York (1984-2008); all incorporated herein by reference in their entirety for all purposes.

The presence or (concentration) level of the serological marker can be detected (e.g., determined, measured, analyzed, etc.) with a hybridization assay, amplification-based assay, immunoassay, immunohistochemical assay, or a combination thereof. Non-limiting examples of assays, techniques, and kits for detecting or determining the presence or level of one or more serological markers in a sample are described in Section VII below.

In other embodiments, the method of diagnosing UC is performed in an individual with symptoms of UC. In additional embodiments, the symptoms of UC include, but are not limited to, rectal inflammation, rectal bleeding, rectal pain, diarrhea, abdominal cramps, abdominal pain, fatigue, weight loss, fever, colon rupture, and combinations thereof.

In some embodiments, the method of diagnosing UC entails analysis of a biological sample selected from the group consisting of whole blood, tissue, saliva, cheek cells, hair, fluid, plasma, serum, cerebrospinal fluid, buccal swabs, mucus, urine, stools, spermatozoids, vaginal secretions, lymph, amniotic fluid, pleural liquid, tears, and combinations thereof.

In other aspects, the present invention provides a method for differentiating between ulcerative colitis (UC) and Crohn's disease (CD) in an individual diagnosed with IBD and/or suspected of having UC. In particular embodiments, the method involves the steps of: (i) analyzing a biological sample obtained from the individual to determine the presence or absence of one or more variant alleles in the GLI1 and/or MDR1 genes; and (ii) associating the presence of the variant allele with a diagnosis of UC.

In particular embodiments, the method of differentiating between UC and CD involves detection of the presence or absence of the GLI1 (rs2228224) variant allele. In other embodiments, the method of differentiating between UC and CD involves detection of the presence or absence of the MDR1 (rs2032582) variant allele. In preferred embodiments, the detection of the presence of the GLI1 (rs2228224) and/or MDR1 (rs2032582) variant alleles is indicative of UC and not indicative of CD.

In other embodiments, the method of differentiating between UC and CD employs an additional step of analyzing the biological sample for the presence or level of a serological marker, wherein detection of the presence or level of the serological marker in conjunction with the presence of one or more variant alleles further improves the differentiation between the UC and CD subtypes of IBD.

In yet other embodiments, the method of differentiating between UC and CD employs detection of a serological marker selected from the group consisting of an anti-neutrophil antibody, an anti-Saccharomyces cerevisiae antibody, an antimicrobial antibody, an acute phase protein, an apolipoprotein, a defensin, a growth factor, a cytokine, a cadherin, and any combination of the markers described herein. Non-limiting examples of serological markers are described herein.

In additional embodiments, the method of differentiating between UC and CD involves analysis of a biological sample. In some embodiments, the biological sample can be obtained from blood, tissue, saliva, cheek cells, hair, fluid, plasma, serum, cerebrospinal fluid, buccal swabs, mucus, urine, stools, spermatozoids, vaginal secretions, lymph, amniotic fluid, pleural liquid, tears, and combinations thereof.

The presence or absence of a variant allele in a genetic marker can be determined using an assay described in Section VI below. Assays that can be used to determine variant allele status include, but are not limited to, electrophoretic analysis assays, restriction length polymorphism analysis assays, sequence analysis assays, hybridization analysis assays, PCR analysis assays, allele-specific hybridization, oligonucleotide ligation allele-specific elongation/ligation, allele-specific amplification, single-base extension, molecular inversion probe, invasive cleavage, selective termination, restriction length polymorphism, sequencing, single strand conformation polymorphism (SSCP), single strand chain polymorphism, mismatch-cleaving, denaturing gradient gel electrophoresis, and combinations thereof.

In yet further additional embodiments, the method of differentiating between UC and CD is performed in a patient with symptoms of UC. In additional embodiments, the symptoms of UC include, but are not limited to, rectal inflammation, rectal bleeding, rectal pain, diarrhea, abdominal cramps, abdominal pain, fatigue, weight loss, fever, colon rupture, and combinations thereof.

In other embodiments, the present invention provides methods for detecting the association of at least one allelic variant in one or more genes selected from GLI1, MDR1, or ATG16L1 with the presence of ulcerative colitis (UC) in a group of individuals. In some specific embodiments, the method comprises: (i) obtaining biological samples from a group of individuals diagnosed with IBD and/or suspected of having UC; (ii) screening the biological samples to determine the presence or absence of a variant allele selected from GLI1 (rs2228224), GLI1 (rs2228226), MDR1 (rs2032582), ATG16L1 (rs2241880), or a combination thereof; and (iii) evaluating whether one or more of the allelic variants show a statistically significant skewed genotype distribution that is skewed towards a group of individuals diagnosed with IBD and/or suspected of having UC, wherein the comparison is between a group of individuals diagnosed with IBD and/or suspected of having UC and a group of healthy individuals.

In more preferred embodiments, the method for detecting the association of at least one allelic variant in one or more genes selected from GLI1, MDR1, or ATG16L1 with the presence of UC in a group of individuals entails detection of the GLI1 (rs2228224) variant allele. In some embodiments, the method entails detection of the GLI1 (rs2228226) variant allele. In other embodiments, the method entails detection of the MDR1 (rs2032582) variant allele. In yet other embodiments, the method entails detection of the ATG16L1 (rs2241880) variant allele. In further embodiments, the method of the invention entails detection of one, two, three, or more variant alleles selected from the group consisting of GLI1 (rs2228224), GLI1 (rs2228226), MDR1 (rs2032582), and ATG16L1 (rs2241880).

In other embodiments, the method for detecting the association of at least one allelic variant in one or more genes selected from GLI1, MDR1, or ATG16L1 with the presence of UC in a group of individuals entails detection of the allelic variant in a biological sample. In yet other embodiments, the biological is selected from blood, tissue, saliva, cheek cells, hair, fluid, plasma, serum, cerebrospinal fluid, buccal swabs, mucus, urine, stools, spermatozoids, vaginal secretions, lymph, amniotic fluid, pleural liquid, tears, and combinations thereof.

In other preferred embodiments, the method for detecting the association of at least one allelic variant in one or more genes selected from GLI1, MDR1, or ATG16L1 with the presence of UC is performed in human populations of individuals diagnosed with IBD and/or suspected of having UC and populations of control individuals.

In additional embodiments, the method for detecting the association of at least one allelic variant in one or more genes selected from GLI1, MDR1, or ATG16L1 with the presence of UC involves screening for the presence or absence of the variant allele. In yet additional embodiments, screening is performed using an assay selected from the group consisting of electrophoretic analysis assays, restriction length polymorphism analysis assays, sequence analysis assays, hybridization analysis assays, PCR analysis assays, allele-specific hybridization, oligonucleotide ligation allele-specific elongation/ligation, allele-specific amplification, single-base extension, molecular inversion probe, invasive cleavage, selective termination, restriction length polymorphism, sequencing, single strand conformation polymorphism (SSCP), single strand chain polymorphism, mismatch-cleaving, denaturing gradient gel electrophoresis, and combinations thereof.

In additional embodiments, the screening is carried out on each individual of a group at one or more allelic variants selected from GLI1 (rs2228224), GLI1 (rs2228226), MDR1 (rs2032582), ATG16L1 (rs2241880), and combinations thereof. In yet additional embodiments, screening is carried out on pools of individuals and pools of controls.

In further embodiments, the method for detecting the association of at least one allelic variant in one or more genes selected from GLI1, MDR1, or ATG16L1 with the presence of UC further entails evaluating whether the allelic variant shows a statistically significant skewed genotype distribution. In yet further embodiments, evaluating consists of evaluating one allelic variant selected from the group consisting of GLI1 (rs2228224), GLI1 (rs2228226), MDR1 (rs2032582), and ATG16L1 (rs2241880) for its distribution in control versus UC populations to determine whether there is a correlation between the presence of absence of the variant allele and presence or absence of UC (e.g., as exemplified in the Examples section below). In yet other further embodiments, the genotype distribution compares more than one allelic variant selected from the group consisting of GLI1 (rs2228224), GLI1 (rs2228226), MDR1 (rs2032582), and ATG16L1 (rs2241880) between control and populations of individuals diagnosed with IBD and/or suspected of having UC. In some embodiments, the genotype distribution is compared using an odds ratio analysis between the individual pools and control pools.

In some embodiments, the present invention also provides kits containing nucleic acid probes specific for one or more allelic variants selected from GLI1 (rs2228224), GLI1 (rs2228226), MDR1 (rs2032582) and ATG16L1 (rs2241880). In particular embodiments, the kit may contain one or more probes selected from the group consisting of:

(SEQ ID NO: 39)
TACCAGAGTCCCAAGTTTCTGGGGG[A/G]TTCCCAGGTTAGCCCAAGCCGTGCT;
(SEQ ID NO: 40)
TATTTAGTTTGACTCACCTTCCCAG[C/A]ACCTTCTAGTTCTTTCTTATCTTTC;
(SEQ ID NO: 41)
TATTTAGTTTGACTCACCTTCCCAG[C/T]ACCTTCTAGTTCTTTCTTATCTTTC;
and
(SEQ ID NO: 42)
CCCAGTCCCCCAGGACAATGTGGAT[A/G]CTCATCCTGGTTCTGGTAAAGAAGT.

In some other embodiments, the present invention also provides an array containing nucleic acid probes specific for one or more allelic variants selected from GLI1 (rs2228224), GLI1 (rs2228226), MDR1 (rs2032582), and ATG16L1 (rs2241880). In other embodiments, an array may contain one or more probes selected from the group consisting of

(SEQ ID NO: 39)
TACCAGAGTCCCAAGTTTCTGGGGG[A/G]TTCCCAGGTTAGCCCAAGCCGTGCT;
(SEQ ID NO: 40)
TATTTAGTTTGACTCACCTTCCCAG[C/A]ACCTTCTAGTTCTTTCTTATCTTTC;
(SEQ ID NO: 41)
TATTTAGTTTGACTCACCTTCCCAG[C/T]ACCTTCTAGTTCTTTCTTATCTTTC;
and
(SEQ ID NO: 42)
CCCAGTCCCCCAGGACAATGTGGAT[A/G]CTCATCCTGGTTCTGGTAAAGAAGT.

In further aspects, a panel for measuring one or more of the markers described herein may be constructed to provide relevant information related to the approach of the invention for diagnosing UC or differentiating between UC and CD. Such a panel may be constructed to detect or determine the presence (or absence) or level of at least 1, 2, 3, 4, 5, 6, 7, 8, 9, 10, 11, 12, 13, 14, 15, 16, 17, 18, 19, 20, 25, 30, 35, 40, or more individual markers such as the genetic, biochemical, serological, protein, or other markers described herein. The analysis of a single marker or subsets of markers can also be carried out by one skilled in the art in various clinical settings. These include, but are not limited to, ambulatory, urgent care, critical care, intensive care, monitoring unit, inpatient, outpatient, physician office, medical clinic, and health screening settings.

In some embodiments, the analysis of markers could be carried out in a variety of physical formats. For example, microtiter plates or automation could be used to facilitate the processing of large numbers of test samples. Alternatively, single sample formats could be developed to facilitate treatment, diagnosis, and prognosis in a timely fashion.

### IV. Inflammatory Bowel Disease

In certain embodiments, the present invention provides methods and systems for diagnosing the ulcerative colitis (UC) subtype of inflammatory bowel disease (IBD). In certain other embodiments, the present invention provides methods and systems for differentiating between UC and other IBD subtypes such as Crohn's disease (CD).

A. Crohn's Disease

Crohn's disease (CD) is a disease of chronic inflammation that can involve any part of the gastrointestinal tract. Commonly, the distal portion of the small intestine, i.e., the ileum, and the cecum are affected. In other cases, the disease is confined to the small intestine, colon, or anorectal region. CD occasionally involves the duodenum and stomach, and more rarely the esophagus and oral cavity.

The variable clinical manifestations of CD are, in part, a result of the varying anatomic localization of the disease. The most frequent symptoms of CD are abdominal pain, diarrhea, and recurrent fever. CD is commonly associated with intestinal obstruction or fistula, an abnormal passage between diseased loops of bowel. CD also includes complications such as inflammation of the eye, joints, and skin, liver disease, kidney stones, and amyloidosis. In addition, CD is associated with an increased risk of intestinal cancer.

Several features are characteristic of the pathology of CD. The inflammation associated with CD, known as transmural inflammation, involves all layers of the bowel wall. Thickening and edema, for example, typically also appear throughout the bowel wall, with fibrosis present in long-standing forms of the disease. The inflammation characteristic of CD is discontinuous in that segments of inflamed tissue, known as “skip lesions,” are separated by apparently normal intestine. Furthermore, linear ulcerations, edema, and inflammation of the intervening tissue lead to a “cobblestone” appearance of the intestinal mucosa, which is distinctive of CD.

A hallmark of CD is the presence of discrete aggregations of inflammatory cells, known as granulomas, which are generally found in the submucosa. Some CD cases display typical discrete granulomas, while others show a diffuse granulomatous reaction or a nonspecific transmural inflammation. As a result, the presence of discrete granulomas is indicative of CD, although the absence of granulomas is also consistent with the disease. Thus, transmural or discontinuous inflammation, rather than the presence of granulomas, is a preferred diagnostic indicator of CD (Rubin and Farber, Essential Pathology (Third Edition), Philadelphia, Lippincott Williams & Wilkins (2001)).

Crohn's disease may be categorized by the behavior of disease as it progresses. This was formalized in the Vienna classification of Crohn's disease. See, Gasche et al., Inflamm. Bowel Dis., 6:8-15 (2000). There are three categories of disease presentation in Crohn's disease: (1) stricturing, (2) penetrating, and (3) inflammatory. Stricturing disease causes narrowing of the bowel which may lead to bowel obstruction or changes in the caliber of the feces. Penetrating disease creates abnormal passageways (fistulae) between the bowel and other structures such as the skin. Inflammatory disease (also known as non-stricturing, non-penetrating disease) causes inflammation without causing strictures or fistulae.

As such, Crohn's disease represents a number of heterogeneous disease subtypes that affect the gastrointestinal tract and may produce similar symptoms. As used herein in reference to CD, the term “clinical subtype” includes a classification of CD defined by a set of clinical criteria that distinguish one classification of CD from another. As non-limiting examples, subjects with CD can be classified as having stricturing (e.g., internal stricturing), penetrating (e.g., internal penetrating), or inflammatory disease as described herein, or these subjects can additionally or alternatively be classified as having fibrostenotic disease, small bowel disease, internal perforating disease, perianal fistulizing disease, UC-like disease, the need for small bowel surgery, the absence of features of UC, or combinations thereof.

In certain instances, subjects with CD can be classified as having complicated CD, which is a clinical subtype characterized by stricturing or penetrating phenotypes. In certain other instances, subjects with CD can be classified as having a form of CD characterized by one or more of the following complications: fibrostenosis, internal perforating disease, and the need for small bowel surgery. In further instances, subjects with CD can be classified as having an aggressive form of fibrostenotic disease requiring small bowel surgery. Criteria relating to these subtypes have been described, for example, in Gasche et al., Inflamm. Bowel Dis., 6:8-15 (2000); Abreu et al., Gastroenterology, 123:679-688 (2002); Vasiliauskas et al., Gut, 47:487-496 (2000); Vasiliauskas et al., Gastroenterology, 110:1810-1819 (1996); and Greenstein et al., Gut, 29:588-592 (1988).

The “fibrostenotic subtype” of CD is a classification of CD characterized by one or more accepted characteristics of fibrostenosing disease. Such characteristics of fibrostenosing disease include, but are not limited to, documented persistent intestinal obstruction or an intestinal resection for an intestinal obstruction. The fibrostenotic subtype of CD can be accompanied by other symptoms such as perforations, abscesses, or fistulae, and can further be characterized by persistent symptoms of intestinal blockage such as nausea, vomiting, abdominal distention, and inability to eat solid food. Intestinal X-rays of patients with the fibrostenotic subtype of CD can show, for example, distention of the bowel before the point of blockage.

The requirement for small bowel surgery in a subject with the fibrostenotic subtype of CD can indicate a more aggressive form of this subtype. Additional subtypes of CD are also known in the art and can be identified using defined clinical criteria. For example, internal perforating disease is a clinical subtype of CD defined by current or previous evidence of entero-enteric or entero-vesicular fistulae, intra-abdominal abscesses, or small bowel perforation. Perianal perforating disease is a clinical subtype of CD defined by current or previous evidence of either perianal fistulae or abscesses or rectovaginal fistula. The UC-like clinical subtype of CD can be defined by current or previous evidence of left-sided colonic involvement, symptoms of bleeding or urgency, and crypt abscesses on colonic biopsies. Disease location can be classified based on one or more endoscopic, radiologic, or pathologic studies.

One skilled in the art understands that overlap can exist between clinical subtypes of CD and that a subject having CD can have more than one clinical subtype of CD. For example, a subject having CD can have the fibrostenotic subtype of CD and can also meet clinical criteria for a clinical subtype characterized by the need for small bowel surgery or the internal perforating disease subtype. Similarly, the markers described herein can be associated with more than one clinical subtype of CD.

B. Ulcerative Colitis

Ulcerative colitis (UC) is a disease of the large intestine characterized by chronic diarrhea with cramping, abdominal pain, rectal bleeding, loose discharges of blood, pus, and mucus. The manifestations of UC vary widely. A pattern of exacerbations and remissions typifies the clinical course for about 70% of UC patients, although continuous symptoms without remission are present in some patients with UC. Local and systemic complications of UC include arthritis, eye inflammation such as uveitis, skin ulcers, and liver disease. In addition, UC, and especially the long-standing, extensive form of the disease is associated with an increased risk of colon carcinoma.

UC is a diffuse disease that usually extends from the most distal part of the rectum for a variable distance proximally. The term “left-sided colitis” describes an inflammation that involves the distal portion of the colon, extending as far as the splenic flexure. Sparing of the rectum or involvement of the right side (proximal portion) of the colon alone is unusual in UC. The inflammatory process of UC is limited to the colon and does not involve, for example, the small intestine, stomach, or esophagus. In addition, UC is distinguished by a superficial inflammation of the mucosa that generally spares the deeper layers of the bowel wall. Crypt abscesses, in which degenerated intestinal crypts are filled with neutrophils, are also typical of UC (Rubin and Farber, supra).

In certain instances, with respect to UC, the variability of symptoms reflect differences in the extent of disease (i.e., the amount of the colon and rectum that are inflamed) and the intensity of inflammation. Disease starts at the rectum and moves “up” the colon to involve more of the organ. UC can be categorized by the amount of colon involved. Typically, patients with inflammation confined to the rectum and a short segment of the colon adjacent to the rectum have milder symptoms and a better prognosis than patients with more widespread inflammation of the colon.

In comparison with CD, which is a patchy disease with frequent sparing of the rectum, UC is characterized by a continuous inflammation of the colon that usually is more severe distally than proximally. The inflammation in UC is superficial in that it is usually limited to the mucosal layer and is characterized by an acute inflammatory infiltrate with neutrophils and crypt abscesses. In contrast, CD affects the entire thickness of the bowel wall with granulomas often, although not always, present. Disease that terminates at the ileocecal valve, or in the colon distal to it, is indicative of UC, while involvement of the terminal ileum, a cobblestone-like appearance, discrete ulcers, or fistulas suggests CD.

The different types of ulcerative colitis are classified according to the location and the extent of inflammation. As used herein in reference to UC, the term “clinical subtype” includes a classification of UC defined by a set of clinical criteria that distinguish one classification of UC from another. As non-limiting examples, subjects with UC can be classified as having ulcerative proctitis, proctosigmoiditis, left-sided colitis, pancolitis, fulminant colitis, and combinations thereof. Criteria relating to these subtypes have been described, for example, in Kornbluth et al., Am. J. Gastroenterol., 99: 1371-85 (2004).

Ulcerative proctitis is a clinical subtype of UC defined by inflammation that is limited to the rectum. Proctosigmoiditis is a clinical subtype of UC which affects the rectum and the sigmoid colon. Left-sided colitis is a clinical subtype of UC which affects the entire left side of the colon, from the rectum to the place where the colon bends near the spleen and begins to run across the upper abdomen (the splenic flexure). Pancolitis is a clinical subtype of UC which affects the entire colon. Fulminant colitis is a rare, but severe form of pancolitis. Patients with fulminant colitis are extremely ill with dehydration, severe abdominal pain, protracted diarrhea with bleeding, and even shock.

In some embodiments, classification of the clinical subtype of UC is important in planning an effective course of treatment. While ulcerative proctitis, proctosigmoiditis, and left-sided colitis can be treated with local agents introduced through the anus, including steroid-based or other enemas and foams, pancolitis must be treated with oral medication so that active ingredients can reach all of the affected portions of the colon.

One skilled in the art understands that overlap can exist between clinical subtypes of UC and that a subject having UC can have more than one clinical subtype of UC. Similarly, the markers described herein can be associated with more than one clinical subtype of UC.

C. Indeterminate Colitis

Indeterminate colitis (IC) is a clinical subtype of IBD that includes both features of CD and UC. Such an overlap in the symptoms of both diseases can occur temporarily (e.g., in the early stages of the disease) or persistently (e.g., throughout the progression of the disease) in patients with IC. Clinically, IC is characterized by abdominal pain and diarrhea with or without rectal bleeding. For example, colitis with intermittent multiple ulcerations separated by normal mucosa is found in patients with the disease. Histologically, there is a pattern of severe ulceration with transmural inflammation. The rectum is typically free of the disease and the lymphoid inflammatory cells do not show aggregation. Although deep slit-like fissures are observed with foci of myocytolysis, the intervening mucosa is typically minimally congested with the preservation of goblet cells in patients with IC.

### V. IBD Markers

A variety of IBD markers, including biochemical markers, serological markers, protein markers, genetic markers, and other clinical or echographic characteristics, are suitable for use in the methods of the present invention for diagnosing IBD, diagnosing UC and differentiating between UC and CD. In certain aspects, the diagnostic and prognostic methods described herein utilize the application of an algorithm (e.g., statistical analysis) to the presence, concentration level, or genotype determined for one or more of the IBD markers to aid or assist in the diagnosis of IBD, the diagnosis of UC, and/or to facilitate differentiation between UC and CD.

Non-limiting examples of IBD markers include: (i) genetic markers such as, e.g., any of the genes set forth in Tables 1-2 (e.g., GLI1, MDR1, and/or ATG16L1) and the NOD2 gene; and (ii) biochemical, serological, and protein markers such as, e.g., cytokines, growth factors, anti-neutrophil antibodies, anti-Saccharomyces cerevisiae antibodies, antimicrobial antibodies, acute phase proteins, apolipoproteins, defensins, cadherins, cellular adhesion molecules, and combinations thereof.

A. Genetic Markers

The determination of the presence or absence of allelic variants in one or more genetic markers in a sample is particularly useful in the present invention. Non-limiting examples of genetic markers include, but are not limited to, any of the genes set forth in Tables 1 and 2. In preferred embodiments, the presence or absence of at least one single nucleotide polymorphism (SNP) in the GLI1, MDR1, and/or ATG16L1 genes is determined. See, e.g., Barrett et al., Nat. Genet., 40:955-62 (2008) and Wang et al., Amer. J. Hum. Genet., 84:399-405 (2009), the disclosures of which are hereby incorporated by reference in their entirety for all purposes.

Table 1 provides an exemplary list of genes wherein genotyping for the presence or absence of one or more allelic variants (e.g., SNPs) therein is useful in the diagnosis of UC. Table 2 provides an exemplary list of genetic markers and corresponding SNPs that find use in differentiating between UC and CD.

TABLE 1
Ulcerative Colitis SNPs

| Gene | SNP |
|---|---|
| GLI1 | rs2228224 |
| MDR1 | rs2032582 |
| ATG16L1 | rs224180 |

TABLE 2
Ulcerative Colitis vs. Crohn's Disease SNPs

| Gene | SNP |
|---|---|
| GLI1 | rs2228224 |
| MDR1 | rs2032582 |

1. GLI1

The Gli proteins are involved in the Hedgehog (Hh) signaling pathway. These proteins have been shown to be involved in cell fate determination, proliferation and patterning in many cell types and most organs during embryo development (see, e.g., Altaba et al., Development 126(14):3205-16 (1999)). The Gli genes act as transcription factors and containing zinc finger binding domains. Specifically, GLI1 (also known as glioma associated oncogene homolog 1) is involved as a transcription factor in the hedgehog signaling pathway and contains C2-H2 zinc fingers domains and a consensus histidine/cysteine linker sequence between zinc fingers. In humans, GLI1 is known to encode an oncogene, and may act as both an inhibitor as well as an activator of transcription (see, e.g., Jacob et al., EMBO Rep. 4(8):761-765 (2003). Some of the downstream gene targets of human GLI1 include regulators of the cell cycle and apoptosis such as cyclin D2 and plakoglobin, respectively (see, e.g., Yoon et al., J. Biol. Chem. 277:5548-5555 (2002)). GLI1 also upregulates FoxM1 in basal cell carcinomas (BCCs) (see, e.g., Teh et al., Cancer Res. 62(16):4773-4780 (2002)). GLI1 expression can also mimic Shh expression in certain cell types (see, e.g., Dahmane et al., Nature 389:876-881 (1997)).

The determination of the presence of absence of allelic variants such as SNPs in the GLI1 (Gli1) gene is particularly useful in the present invention. As used herein, the term “GLI1 variant” or variants thereof includes a nucleotide sequence of a GLI1 gene containing one or more changes as compared to the wild-type GLI1 gene or an amino acid sequence of a GLI1 polypeptide containing one or more changes as compared to the wild-type GLI1 polypeptide sequence. GLI1 has been localized to be within the IBD2 linkage region chromosome 12 (12q13). The rs2228226 SNP, which is a transition from C to G (located in Exon 12 of GLI1) mutation, was identified as a germline variation in GLI1 in patients with IBD (see, e.g., Lees et al., PLOS 5(12):1761-1775 (2008)). The rs2228226 mutation in GLI1 produces a protein with reduced function. See, e.g., Lees, supra and Bentley et al., Genes Immun. (May 2010).

Gene location information for GL1 is set forth in, e.g., GeneID:2735. The mRNA (coding) and polypeptide sequences of human GLI1 are set forth in, e.g., NM_{—}005269.2 (SEQ ID NO:25) and NP_{—}005260.1 (SEQ ID NO:26), respectively. In addition, the complete sequence of human chromosome 12, GRCh37 primary reference assembly, which includes GLI1, is set forth in, e.g., GenBank Accession No. NC_{—}000012.11. Furthermore, the sequence of GLI1 from other species can be found in the GenBank database.

The rs2228224 SNP is particularly useful in the methods of the present invention and is located at nucleotide position 2672 of GenBank Accession Number NM_{—}001160045.1 (SEQ ID NO:37), as a G to A transition, corresponding to a change from a glycine to an aspartic acid at position 805 of GenBank Accession Number NP_{—}001153517.1 (SEQ ID NO:38); position 2753 of GenBank Accession Number NM_{—}001167609.1 (SEQ ID NO:35), as a G to A transition, corresponding to a change from a glycine to an aspartic acid at position 892 of GenBank Accession Number NP_{—}001161081.1 (SEQ ID NO:36); or position 2876 of GenBank Accession Number NM_{—}005269.2 (SEQ ID NO:25), as a G to A transition, corresponding to a change from a glycine to an aspartic acid at position 933 of GenBank Accession Number NP_{—}005260.1 (SEQ ID NO:26).

The rs2228226 SNP is located at nucleotide position 3172 of GenBank Accession Number NM_{—}001160045.1 (SEQ ID NO:33), as a G to C transversion, corresponding to a change from a glutamic acid to a glutamine at position 972 of GenBank Accession Number NP_{—}001153517.1 (SEQ ID NO:34); position 3253 of GenBank Accession Number NM_{—}001167609.1 (SEQ ID NO:35), as a G to C transversion, corresponding to a change from a glutamic acid to a glutamine at position 1059 of GenBank Accession Number NP_{—}001161081.1 (SEQ ID NO:36); or position 3376 of GenBank Accession Number NM_{—}005269.2 (SEQ ID NO:37), as a G to C transversion, corresponding to a change from a glutamic acid to a glutamine at position 1100 of GenBank Accession Number NP_{—}005260.1 (SEQ ID NO:38).

2. MDR1

MDR1 is a member of the ATP-binding cassette (ABC) transporter family of proteins. MDR1 is also known as multi-drug resistance or ATP-binding cassette, sub-family B (MDR/TAP) member 1 (ABCB1), P-glycoprotein (permeability-glycoprotein), and PGY1. ABC proteins transport a variety of molecules across both extracellular and intracellular membranes. There are seven distinct subfamilies of ABC transports: ABC1, MDR/TAP, MRP, ALD, OABP, GCN20 and White. MDR1 is member of the MDR/TAP family and these proteins are involved in multidrug resistance. MDR1 is involved specifically in the decreased drug accumulation in multi-drug resistant cells and can mediate resistance to anticancer drugs. MDR1 functions as a transporter in the blood-brain barrier, working as an ATP-dependent efflux pump for a variety of substances. See., e.g., Aller et al., Science 323 (5922):1718-22 (2009); van Helvoort, et al., Cell 87(3):507-517 (1996); Ueda et al., J. Biol. Chem. 262 (2):505-508 (1987); and Thiebaut et al., PNAS 84(21):7735-7738 (1987).

The determination of the presence of absence of allelic variants such as SNPs in the MDR1 gene is particularly useful in the present invention. As used herein, the term “MDR1 variant” or variants thereof includes a nucleotide sequence of a MDR1 gene containing one or more changes as compared to the wild-type MDR1 gene or an amino acid sequence of a MDR1 polypeptide containing one or more changes as compared to the wild-type MDR1 polypeptide sequence. MDR1 has been localized to human chromosome 7. MDR1 is a membrane transporter protein for which human polymorphisms have been reported in Ala893Ser/Thr and C3435T that alter pharmacokinetic profiles for a variety of drugs. See, e.g., Brant et al., Am. J. Hum. Genet. 73:1282-1292 (2003) and Wang et al., Curr. Pharmacogenomics and Personalized Medicine 7:40-58 (2009).

Gene location information for MDR1 is set forth in, e.g., GeneID: 5243. The mRNA (coding) and polypeptide sequences of human MDR1 are set forth in, e.g., NM_{—}000927.3 (SEQ ID NO:27) and NP_{—}000918.2 (SEQ ID NO:28) respectively. In addition, the complete sequence of human chromosome 7 (7q21.12), GRCh37 primary reference assembly, which includes MDR1, is set forth in, e.g., GenBank Accession No. NT_{—}007933.15. Furthermore, the sequence of MDR1 from other species can be found in the GenBank database.

The rs2032582 SNP is particularly useful in the methods of the present invention and is located at nucleotide position 3095 of SEQ ID NO:27 (NM_{—}000927.3), as either a T to A transversion or a T to G transversion. The T to A transversion corresponds to a change from a serine to a threonine at position 893 of SEQ ID NO:28 (NP_{—}000918.2), whereas the T to G transversion corresponds to a change from a serine to an alanine at position 893 of SEQ ID NO:28 (NP_{—}000918.2).

3. ATG16L1

ATG16L1, also known as autophagy related 16-like 1, is a protein involved the intracellular process of delivering cytoplasmic components to lysosomes, a process called autophagy. Autophagy is a process used by cells to recycle cellular components. Autophagy processes are also involved in the inflammatory response and facilitates immune system destruction of bacteria. The ATG16L1 protein is a WD repeated containing component of a large protein complex and associates with the autophagic isolation membrane throughout autophagosome formation (see, e.g., Mizushima et al., Journal of Cell Science 116(9):1679-1688 (2003) and Hampe et al., Nature Genetics 39:207-211 (2006)). ATG16L1 has been implicated in Crohn's Disease (see, e.g., Rioux et al., Nature Genetics 39(5):596-604 (2007)). See also, e.g., Márquez et al., Inflamm. Bowel Disease 15(11):1697-1704 (2009); Mizushima et al., J. Cell Science 116:1679-1688 (2003); and Zheng et al., DNA Sequence: The J of DNA Sequencing and Mapping 15(4): 303-5 (2004)).

The determination of the presence of absence of allelic variants such as SNPs in the ATG16L1 gene is particularly useful in the present invention. As used herein, the term “ATG16L1 variant” or variants thereof includes a nucleotide sequence of an ATG16L1 gene containing one or more changes as compared to the wild-type ATG16L1 gene or an amino acid sequence of an ATG16L1 polypeptide containing one or more changes as compared to the wild-type ATG16L1 polypeptide sequence. ATG16L1, also known as autophagy related 16-like 1, has been localized to human chromosome 2.

Gene location information for ATG16L1 is set forth in, e.g., GeneID:55054. The mRNA (coding) and polypeptide sequences of human ATG16L1 are set forth in, e.g., NM_{—}017974.3 (SEQ ID NO:29) or NM_{—}030803.6 (SEQ ID NO:31) and NP_{—}060444.3 (SEQ ID NO:30) or NP_{—}110430.5 (SEQ ID NO:32), respectively. In addition, the complete sequence of human chromosome 2 (2q37.1), GRCh37 primary reference assembly, which includes ATG16L1, is set forth in, e.g., GenBank Accession No. NT_{—}005120.16. Furthermore, the sequence of ATG16L1 from other species can be found in the GenBank database.

The rs2241880 SNP is particularly useful in the methods of the present invention and is located at nucleotide position 1098 of SEQ ID NO:29 (NM_{—}017974.3), as an A to G transition, corresponding to a change from threonine to alanine at position 281 of SEQ ID NO:30 (NP_{—}060444.3) or at position 1155 of SEQ ID NO:31 (NM_{—}030803.6), as an A to G transition, corresponding to a change from threonine to alanine at position 300 of SEQ ID NO:32 (NP_{—}110430.5).

B. Cytokines

The determination of the presence or level of at least one cytokine in a sample is useful in the present invention. As used herein, the term “cytokine” includes any of a variety of polypeptides or proteins secreted by immune cells that regulate a range of immune system functions and encompasses small cytokines such as chemokines. The term “cytokine” also includes adipocytokines, which comprise a group of cytokines secreted by adipocytes that function, for example, in the regulation of body weight, hematopoiesis, angiogenesis, wound healing, insulin resistance, the immune response, and the inflammatory response.

In certain aspects, the presence or level of at least one cytokine including, but not limited to, TNF-α, TNF-related weak inducer of apoptosis (TWEAK), osteoprotegerin (OPG), IFN-α, IFN-β, IFN-γ, IL-1α, IL-1β, IL-1 receptor antagonist (IL-1ra), IL-2, IL-4, IL-5, IL-6, soluble IL-6 receptor (sIL-6R), IL-7, IL-8, IL-9, IL-10, IL-12, IL-13, IL-15, IL-17, IL-23, and IL-27 is determined in a sample. In certain other aspects, the presence or level of at least one chemokine such as, for example, CXCL1/GRO1/GROα, CXCL2/GRO2, CXCL3/GRO3, CXCL4/PF-4, CXCL5/ENA-78, CXCL6/GCP-2, CXCL7/NAP-2, CXCL9/MIG, CXCL10/IP-10, CXCL11/I-TAC, CXCL12/SDF-1, CXCL13/BCA-1, CXCL14/BRAK, CXCL15, CXCL16, CXCL17/DMC, CCL1, CCL2/MCP-1, CCL3/MIP-1α, CCL4/MIP-1β, CCL5/RANTES, CCL6/C10, CCL7/MCP-3, CCL8/MCP-2, CCL9/CCL10, CCL11/Eotaxin, CCL12/MCP-5, CCL13/MCP-4, CCL14/HCC-1, CCL15/MIP-5, CCL16/LEC, CCL17/TARC, CCL18/MIP-4, CCL19/MIP-3β, CCL20/MIP-3α, CCL21/SLC, CCL22/MDC, CCL23/MPIF1, CCL24/Eotaxin-2, CCL25/TECK, CCL26/Eotaxin-3, CCL27/CTACK, CCL28/MEC, CL1, CL2, and CX₃CL1 is determined in a sample. In certain further aspects, the presence or level of at least one adipocytokine including, but not limited to, leptin, adiponectin, resistin, active or total plasminogen activator inhibitor-1 (PAI-1), visfatin, and retinol binding protein 4 (RBP4) is determined in a sample. Preferably, the presence or level of IL-6, IL-1β, and/or TWEAK is determined.

In certain instances, the presence or level of a particular cytokine is detected at the level of mRNA expression with an assay such as, for example, a hybridization assay or an amplification-based assay. In certain other instances, the presence or level of a particular cytokine is detected at the level of protein expression using, for example, an immunoassay (e.g., ELISA) or an immunohistochemical assay. Suitable ELISA kits for determining the presence or level of a cytokine such as IL-6, IL-1β, or TWEAK in a serum, plasma, saliva, or urine sample are available from, e.g., R&D Systems, Inc. (Minneapolis, Minn.), Neogen Corp. (Lexington, Ky.), Alpco Diagnostics (Salem, N.H.), Assay Designs, Inc. (Ann Arbor, Mich.), BD Biosciences Pharmingen (San Diego, Calif.), Invitrogen (Camarillo, Calif.), Calbiochem (San Diego, Calif.), CHEMICON International, Inc. (Temecula, Calif.), Antigenix America Inc. (Huntington Station, N.Y.), QIAGEN Inc. (Valencia, Calif.), Bio-Rad Laboratories, Inc. (Hercules, Calif.), and/or Bender MedSystems Inc. (Burlingame, Calif.).

The human IL-6 polypeptide sequence is set forth in, e.g., Genbank Accession No. NP_{—}000591 (SEQ ID NO:1). The human IL-6 mRNA (coding) sequence is set forth in, e.g., Genbank Accession No. NM_{—}000600 (SEQ ID NO:2). One skilled in the art will appreciate that IL-6 is also known as interferon beta 2 (IFNB2), HGF, HSF, and BSF2.

The human IL-1β polypeptide sequence is set forth in, e.g., Genbank Accession No. NP_{—}000567 (SEQ ID NO:3). The human IL-1β mRNA (coding) sequence is set forth in, e.g., Genbank Accession No. NM_{—}000576 (SEQ ID NO:4). One skilled in the art will appreciate that IL-1β is also known as IL1F2 and IL-1beta.

The human TWEAK polypeptide sequence is set forth in, e.g., Genbank Accession Nos. NP_{—}003800 (SEQ ID NO:5) and AAC51923. The human TWEAK mRNA (coding) sequence is set forth in, e.g., Genbank Accession Nos. NM_{—}003809 (SEQ ID NO:6) and BC104420. One skilled in the art will appreciate that TWEAK is also known as tumor necrosis factor ligand superfamily member 12 (TNFSF12), APO3 ligand (APO3L), CD255, DR3 ligand, growth factor-inducible 14 (Fn14) ligand, and UNQ181/PRO207.

C. Growth Factors

The determination of the presence or level of one or more growth factors in a sample is also useful in the present invention. As used herein, the term “growth factor” includes any of a variety of peptides, polypeptides, or proteins that are capable of stimulating cellular proliferation and/or cellular differentiation.

In certain aspects, the presence or level of at least one growth factor including, but not limited to, epidermal growth factor (EGF), heparin-binding epidermal growth factor (HB-EGF), vascular endothelial growth factor (VEGF), pigment epithelium-derived factor (PEDF; also known as SERPINF1), amphiregulin (AREG; also known as schwannoma-derived growth factor (SDGF)), basic fibroblast growth factor (bFGF), hepatocyte growth factor (HGF), transforming growth factor-α (TGF-α), transforming growth factor-β (TGF-β), bone morphogenetic proteins (e.g., BMP1-BMP15), platelet-derived growth factor (PDGF), nerve growth factor (NGF), β-nerve growth factor (β-NGF), neurotrophic factors (e.g., brain-derived neurotrophic factor (BDNF), neurotrophin 3 (NT3), neurotrophin 4 (NT4), etc.), growth differentiation factor-9 (GDF-9), granulocyte-colony stimulating factor (G-CSF), granulocyte-macrophage colony stimulating factor (GM-CSF), myostatin (GDF-8), erythropoietin (EPO), and thrombopoietin (TPO) is determined in a sample. Preferably, the presence or level of EGF is determined.

In certain instances, the presence or level of a particular growth factor is detected at the level of mRNA expression with an assay such as, for example, a hybridization assay or an amplification-based assay. In certain other instances, the presence or level of a particular growth factor is detected at the level of protein expression using, for example, an immunoassay (e.g., ELISA) or an immunohistochemical assay. Suitable ELISA kits for determining the presence or level of a growth factor such as EGF in a serum, plasma, saliva, or urine sample are available from, e.g., Antigenix America Inc. (Huntington Station, N.Y.), Promega (Madison, Wis.), R&D Systems, Inc. (Minneapolis, Minn.), Invitrogen (Camarillo, Calif.), CHEMICON International, Inc. (Temecula, Calif.), Neogen Corp. (Lexington, Ky.), PeproTech (Rocky Hill, N.J.), Alpco Diagnostics (Salem, N.H.), Pierce Biotechnology, Inc. (Rockford, Ill.), and/or Abazyme (Needham, Mass.).

The human epidermal growth factor (EGF) polypeptide sequence is set forth in, e.g., Genbank Accession No. NP_{—}001954 (SEQ ID NO:7). The human EGF mRNA (coding) sequence is set forth in, e.g., Genbank Accession No. NM_{—}001963 (SEQ ID NO:8). One skilled in the art will appreciate that EGF is also known as beta-urogastrone, URG, and HOMG4.

D. Anti-Neutrophil Antibodies

The determination of ANCA levels and/or the presence or absence of pANCA in a sample is also useful in the present invention. As used herein, the term “anti-neutrophil cytoplasmic antibody” or “ANCA” includes antibodies directed to cytoplasmic and/or nuclear components of neutrophils. ANCA activity can be divided into several broad categories based upon the ANCA staining pattern in neutrophils: (1) cytoplasmic neutrophil staining without perinuclear highlighting (cANCA); (2) perinuclear staining around the outside edge of the nucleus (pANCA); (3) perinuclear staining around the inside edge of the nucleus (NSNA); and (4) diffuse staining with speckling across the entire neutrophil (SAPPA). In certain instances, pANCA staining is sensitive to DNase treatment. The term ANCA encompasses all varieties of anti-neutrophil reactivity, including, but not limited to, cANCA, pANCA, NSNA, and SAPPA. Similarly, the term ANCA encompasses all immunoglobulin isotypes including, without limitation, immunoglobulin A and G.

ANCA levels in a sample from an individual can be determined, for example, using an immunoassay such as an enzyme-linked immunosorbent assay (ELISA) with alcohol-fixed neutrophils (see, e.g., Example 1 of PCT Publication No. WO 2010/120814). The presence or absence of a particular category of ANCA such as pANCA can be determined, for example, using an immunohistochemical assay such as an indirect fluorescent antibody (IFA) assay. In certain embodiments, the presence or absence of pANCA in a sample is determined using an immunofluorescence assay with DNase-treated, fixed neutrophils (see, e.g., Example 2 of PCT Publication No. WO 2010/120814). In addition to fixed neutrophils, antibodies directed against human antibodies can be used for detection. Antigens specific for ANCA are also suitable for determining ANCA levels, including, without limitation, unpurified or partially purified neutrophil extracts; purified proteins, protein fragments, or synthetic peptides such as histone H1 or ANCA-reactive fragments thereof (see, e.g., U.S. Pat. No. 6,074,835); histone H1-like antigens, porin antigens, Bacteroides antigens, or ANCA-reactive fragments thereof (see, e.g., U.S. Pat. No. 6,033,864); secretory vesicle antigens or ANCA-reactive fragments thereof (see, e.g., U.S. patent application Ser. No. 08/804,106); and anti-ANCA idiotypic antibodies. One skilled in the art will appreciate that the use of additional antigens specific for ANCA is within the scope of the present invention. The disclosures of each of the above-described patent documents are hereby incorporated by reference in their entirety for all purposes.

E. Anti-Saccharomyces cerevisiae Antibodies

The determination of the presence or level of ASCA (e.g., ASCA-IgA, ASCA-IgG, ASCA-IgM, etc.) in a sample is also useful in the present invention. The term “anti-Saccharomyces cerevisiae immunoglobulin A” or “ASCA-IgA” includes antibodies of the immunoglobulin A isotype that react specifically with S. cerevisiae. Similarly, the term “anti-Saccharomyces cerevisiae immunoglobulin G” or “ASCA-IgG” includes antibodies of the immunoglobulin G isotype that react specifically with S. cerevisiae.

The determination of whether a sample is positive for ASCA-IgA or ASCA-IgG is made using an antibody specific for human antibody sequences or an antigen specific for ASCA. Such an antigen can be any antigen or mixture of antigens that is bound specifically by ASCA-IgA and/or ASCA-IgG. Although ASCA antibodies were initially characterized by their ability to bind S. cerevisiae, those of skill in the art will understand that an antigen that is bound specifically by ASCA can be obtained from S. cerevisiae or from a variety of other sources so long as the antigen is capable of binding specifically to ASCA antibodies. Accordingly, exemplary sources of an antigen specific for ASCA, which can be used to determine the levels of ASCA-IgA and/or ASCA-IgG in a sample, include, without limitation, whole killed yeast cells such as Saccharomyces or Candida cells; yeast cell wall mannan such as phosphopeptidomannan (PPM); oligosachharides such as oligomannosides; neoglycolipids; anti-ASCA idiotypic antibodies; and the like. Different species and strains of yeast, such as S. cerevisiae strain Su1, Su2, CBS 1315, or BM 156, or Candida albicans strain VW32, are suitable for use as an antigen specific for ASCA-IgA and/or ASCA-IgG. Purified and synthetic antigens specific for ASCA are also suitable for use in determining the levels of ASCA-IgA and/or ASCA-IgG in a sample. Examples of purified antigens include, without limitation, purified oligosaccharide antigens such as oligomannosides. Examples of synthetic antigens include, without limitation, synthetic oligomannosides such as those described in U.S. Patent Publication No. 20030105060, e.g., D-Man β(1-2) D-Man β(1-2) D-Man β(1-2) D-Man-OR, D-Man α(1-2) D-Man α(1-2) D-Man α(1-2) D-Man-OR, and D-Man α(1-3) D-Man α(1-2) D-Man α(1-2) D-Man-OR, wherein R is a hydrogen atom, a C₁ to C₂₀ alkyl, or an optionally labeled connector group.

Preparations of yeast cell wall mannans, e.g., PPM, can be used in determining the levels of ASCA-IgA and/or ASCA-IgG in a sample. Such water-soluble surface antigens can be prepared by any appropriate extraction technique known in the art, including, for example, by autoclaving, or can be obtained commercially (see, e.g., Lindberg et al., Gut, 33:909-913 (1992)). The acid-stable fraction of PPM is also useful in the statistical algorithms of the present invention (Sendid et al., Clin. Diag. Lab. Immunol., 3:219-226 (1996)). An exemplary PPM that is useful in determining ASCA levels in a sample is derived from S. uvarum strain ATCC #38926. Example 3 of PCT Publication No. WO 2010/120814, the disclosure of which is hereby incorporated by reference in its entirety for all purposes, describes the preparation of yeast cell well mannan and an analysis of ASCA levels in a sample using an ELISA assay.

Purified oligosaccharide antigens such as oligomannosides can also be useful in determining the levels of ASCA-IgA and/or ASCA-IgG in a sample. The purified oligomannoside antigens are preferably converted into neoglycolipids as described in, for example, Faille et al., Eur. J. Microbiol. Infect. Dis., 11:438-446 (1992). One skilled in the art understands that the reactivity of such an oligomannoside antigen with ASCA can be optimized by varying the mannosyl chain length (Frosh et al., Proc Natl. Acad. Sci. USA, 82:1194-1198 (1985)); the anomeric configuration (Fukazawa et al., In “Immunology of Fungal Disease,” E. Kurstak (ed.), Marcel Dekker Inc., New York, pp. 37-62 (1989); Nishikawa et al., Microbiol. Immunol., 34:825-840 (1990); Poulain et al., Eur. J. Clin. Microbiol., 23:46-52 (1993); Shibata et al., Arch. Biochem. Biophys., 243:338-348 (1985); Trinel et al., Infect. Immun., 60:3845-3851 (1992)); or the position of the linkage (Kikuchi et al., Planta, 190:525-535 (1993)).

Suitable oligomannosides for use in the methods of the present invention include, without limitation, an oligomannoside having the mannotetraose Man(1-3) Man(1-2) Man(1-2) Man. Such an oligomannoside can be purified from PPM as described in, e.g., Faille et al., supra. An exemplary neoglycolipid specific for ASCA can be constructed by releasing the oligomannoside from its respective PPM and subsequently coupling the released oligomannoside to 4-hexadecylaniline or the like.

F. Anti-Microbial Antibodies

The determination of the presence or level of anti-OmpC antibody in a sample is also useful in the present invention. As used herein, the term “anti-outer membrane protein C antibody” or “anti-OmpC antibody” includes antibodies directed to a bacterial outer membrane porin as described in, e.g., U.S. Pat. No. 7,138,237 and PCT Publication No. WO 01/89361, the disclosures of which are hereby incorporated by reference in their entirety for all purposes. The term “outer membrane protein C” or “OmpC” refers to a bacterial porin that is immunoreactive with an anti-OmpC antibody.

The level of anti-OmpC antibody present in a sample from an individual can be determined using an OmpC protein or a fragment thereof such as an immunoreactive fragment thereof. Suitable OmpC antigens useful in determining anti-OmpC antibody levels in a sample include, without limitation, an OmpC protein, an OmpC polypeptide having substantially the same amino acid sequence as the OmpC protein, or a fragment thereof such as an immunoreactive fragment thereof. As used herein, an OmpC polypeptide generally describes polypeptides having an amino acid sequence with greater than about 50% identity, preferably greater than about 60% identity, more preferably greater than about 70% identity, still more preferably greater than about 80%, 85%, 90%, 95%, 96%, 97%, 98%, or 99% amino acid sequence identity with an OmpC protein, with the amino acid identity determined using a sequence alignment program such as CLUSTALW. Such antigens can be prepared, for example, by purification from enteric bacteria such as E. coli, by recombinant expression of a nucleic acid such as Genbank Accession No. K00541, by synthetic means such as solution or solid phase peptide synthesis, or by using phage display. Example 4 of PCT Publication No. WO 2010/120814, the disclosure of which is hereby incorporated by reference in its entirety for all purposes, describes the preparation of OmpC protein and an analysis of anti-OmpC antibody levels in a sample using an ELISA assay.

The determination of the presence or level of anti-I2 antibody in a sample is also useful in the present invention. As used herein, the term “anti-I2 antibody” includes antibodies directed to a microbial antigen sharing homology to bacterial transcriptional regulators as described in, e.g., U.S. Pat. No. 6,309,643, the disclosure of which is hereby incorporated by reference in its entirety for all purposes. The term “I2” refers to a microbial antigen that is immunoreactive with an anti-I2 antibody. The microbial I2 protein is a polypeptide of 100 amino acids sharing some similarity weak homology with the predicted protein 4 from C. pasteurianum, Rv3557c from Mycobacterium tuberculosis, and a transcriptional regulator from Aquifex aeolicus. The nucleic acid and protein sequences for the I2 protein are described in, e.g., U.S. Pat. No. 6,309,643.

The level of anti-I2 antibody present in a sample from an individual can be determined using an I2 protein or a fragment thereof such as an immunoreactive fragment thereof. Suitable I2 antigens useful in determining anti-I2 antibody levels in a sample include, without limitation, an I2 protein, an I2 polypeptide having substantially the same amino acid sequence as the I2 protein, or a fragment thereof such as an immunoreactive fragment thereof. Such I2 polypeptides exhibit greater sequence similarity to the I2 protein than to the C. pasteurianum protein 4 and include isotype variants and homologs thereof. As used herein, an I2 polypeptide generally describes polypeptides having an amino acid sequence with greater than about 50% identity, preferably greater than about 60% identity, more preferably greater than about 70% identity, still more preferably greater than about 80%, 85%, 90%, 95%, 96%, 97%, 98%, or 99% amino acid sequence identity with a naturally-occurring I2 protein, with the amino acid identity determined using a sequence alignment program such as CLUSTALW. Such I2 antigens can be prepared, for example, by purification from microbes, by recombinant expression of a nucleic acid encoding an I2 antigen, by synthetic means such as solution or solid phase peptide synthesis, or by using phage display. Determination of anti-I2 antibody levels in a sample can be performed using an ELISA assay (see, e.g., Examples 5, 20, and 22 of PCT Publication No. WO 2010/120814, the disclosure of which is hereby incorporated by reference in its entirety for all purposes) or a histological assay.

The determination of the presence or level of anti-flagellin antibody in a sample is also useful in the present invention. As used herein, the term “anti-flagellin antibody” includes antibodies directed to a protein component of bacterial flagella as described in, e.g., U.S. Pat. No. 7,361,733 and PCT Patent Publication No. WO 03/053220, the disclosures of which are hereby incorporated by reference in their entirety for all purposes. The term “flagellin” refers to a bacterial flagellum protein that is immunoreactive with an anti-flagellin antibody. Microbial flagellins include, e.g., proteins found in bacterial flagellum that arrange themselves in a hollow cylinder to form the filament.

The level of anti-flagellin antibody present in a sample from an individual can be determined using a flagellin protein or a fragment thereof such as an immunoreactive fragment thereof. Suitable flagellin antigens useful in determining anti-flagellin antibody levels in a sample include, without limitation, a flagellin protein such as Cbir-1 flagellin, flagellin X, flagellin A, flagellin B, fragments thereof, and combinations thereof, a flagellin polypeptide having substantially the same amino acid sequence as the flagellin protein, or a fragment thereof such as an immunoreactive fragment thereof. As used herein, a flagellin polypeptide generally describes polypeptides having an amino acid sequence with greater than about 50% identity, preferably greater than about 60% identity, more preferably greater than about 70% identity, still more preferably greater than about 80%, 85%, 90%, 95%, 96%, 97%, 98%, or 99% amino acid sequence identity with a naturally-occurring flagellin protein, with the amino acid identity determined using a sequence alignment program such as CLUSTALW. Such flagellin antigens can be prepared, e.g., by purification from bacterium such as Helicobacter Bilis, Helicobacter mustelae, Helicobacter pylori, Butyrivibrio fibrisolvens, and bacterium found in the cecum, by recombinant expression of a nucleic acid encoding a flagellin antigen, by synthetic means such as solution or solid phase peptide synthesis, or by using phage display. Determination of anti-flagellin (e.g., anti-Cbir-1) antibody levels in a sample can be performed by using an ELISA assay or a histological assay.

G. Acute Phase Proteins

The determination of the presence or level of one or more acute-phase proteins in a sample is also useful in the present invention. Acute-phase proteins are a class of proteins whose plasma concentrations increase (positive acute-phase proteins) or decrease (negative acute-phase proteins) in response to inflammation. This response is called the acute-phase reaction (also called acute-phase response). Examples of positive acute-phase proteins include, but are not limited to, C-reactive protein (CRP), D-dimer protein, mannose-binding protein, alpha 1-antitrypsin, alpha 1-antichymotrypsin, alpha 2-macroglobulin, fibrinogen, prothrombin, factor VIII, von Willebrand factor, plasminogen, complement factors, ferritin, serum amyloid P component, serum amyloid A (SAA), orosomucoid (alpha 1-acid glycoprotein, AGP), ceruloplasmin, haptoglobin, and combinations thereof. Non-limiting examples of negative acute-phase proteins include albumin, transferrin, transthyretin, transcortin, retinol-binding protein, and combinations thereof. Preferably, the presence or level of CRP and/or SAA is determined.

In certain instances, the presence or level of a particular acute-phase protein is detected at the level of mRNA expression with an assay such as, for example, a hybridization assay or an amplification-based assay. In certain other instances, the presence or level of a particular acute-phase protein is detected at the level of protein expression using, for example, an immunoassay (e.g., ELISA) or an immunohistochemical assay. For example, a sandwich colorimetric ELISA assay available from Alpco Diagnostics (Salem, N.H.) can be used to determine the level of CRP in a serum, plasma, urine, or stool sample. Similarly, an ELISA kit available from Biomeda Corporation (Foster City, Calif.) can be used to detect CRP levels in a sample. Other methods for determining CRP levels in a sample are described in, e.g., U.S. Pat. Nos. 6,838,250 and 6,406,862; and U.S. Patent Publication Nos. 20060024682 and 20060019410, the disclosures of which are hereby incorporated by reference in their entirety for all purposes. Additional methods for determining CRP levels include, e.g., immunoturbidimetry assays, rapid immunodiffusion assays, and visual agglutination assays.

C-reactive protein (CRP) is a protein found in the blood in response to inflammation (an acute-phase protein). CRP is typically produced by the liver and by fat cells (adipocytes). It is a member of the pentraxin family of proteins. The human CRP polypeptide sequence is set forth in, e.g., Genbank Accession No. NP_{—}000558 (SEQ ID NO:9). The human CRP mRNA (coding) sequence is set forth in, e.g., Genbank Accession No. NM_{—}000567 (SEQ ID NO:10). One skilled in the art will appreciate that CRP is also known as PTX1, MGC88244, and MGC149895.

H. Apolipoproteins

The determination of the presence or level of one or more apolipoproteins in a sample is also useful in the present invention. Apolipoproteins are proteins that bind to fats (lipids). They form lipoproteins, which transport dietary fats through the bloodstream. Dietary fats are digested in the intestine and carried to the liver. Fats are also synthesized in the liver itself. Fats are stored in fat cells (adipocytes). Fats are metabolized as needed for energy in the skeletal muscle, heart, and other organs and are secreted in breast milk. Apolipoproteins also serve as enzyme co-factors, receptor ligands, and lipid transfer carriers that regulate the metabolism of lipoproteins and their uptake in tissues. Examples of apolipoproteins include, but are not limited to, ApoA (e.g., ApoA-I, ApoA-II, ApoA-IV, ApoA-V), ApoB (e.g., ApoB48, ApoB100), ApoC (e.g., ApoC-I, ApoC-II, ApoC-III, ApoC-IV), ApoD, ApoE, ApoH, serum amyloid A (SAA), and combinations thereof. Preferably, the presence or level of SAA is determined.

In certain instances, the presence or level of a particular apolipoprotein is detected at the level of mRNA expression with an assay such as, for example, a hybridization assay or an amplification-based assay. In certain other instances, the presence or level of a particular apolipoprotein is detected at the level of protein expression using, for example, an immunoassay (e.g., ELISA) or an immunohistochemical assay. Suitable ELISA kits for determining the presence or level of SAA in a sample such as serum, plasma, saliva, urine, or stool are available from, e.g., Antigenix America Inc. (Huntington Station, N.Y.), Abazyme (Needham, Mass.), USCN Life (Missouri City, Tex.), and/or U.S. Biological (Swampscott, Mass.).

Serum amyloid A (SAA) proteins are a family of apolipoproteins associated with high-density lipoprotein (HDL) in plasma. Different isoforms of SAA are expressed constitutively (constitutive SAAs) at different levels or in response to inflammatory stimuli (acute phase SAAs). These proteins are predominantly produced by the liver. The conservation of these proteins throughout invertebrates and vertebrates suggests SAAs play a highly essential role in all animals. Acute phase serum amyloid A proteins (A-SAAs) are secreted during the acute phase of inflammation. The human SAA polypeptide sequence is set forth in, e.g., Genbank Accession No. NP_{—}000322 (SEQ ID NO:11). The human SAA mRNA (coding) sequence is set forth in, e.g., Genbank Accession No. NM_{—}000331 (SEQ ID NO:12). One skilled in the art will appreciate that SAA is also known as PIG4, TP5314, MGC111216, and SAA1.

I. Defensins

The determination of the presence or level of one or more defensins in a sample is also useful in the present invention. Defensins are small cysteine-rich cationic proteins found in both vertebrates and invertebrates. They are active against bacteria, fungi, and many enveloped and nonenveloped viruses. They typically consist of 18-45 amino acids, including 6 (in vertebrates) to 8 conserved cysteine residues. Cells of the immune system contain these peptides to assist in killing phagocytized bacteria, for example, in neutrophil granulocytes and almost all epithelial cells. Most defensins function by binding to microbial cell membranes, and once embedded, forming pore-like membrane defects that allow efflux of essential ions and nutrients. Non-limiting examples of defensins include α-defensins (e.g., DEFA1, DEFA1A3, DEFA3, DEFA4), β-defensins (e.g., β defensin-1 (DEFB1), β defensin-2 (DEFB2), DEFB103A/DEFB103B to DEFB107A/DEFB107B, DEFB110 to DEFB133), and combinations thereof. Preferably, the presence or level of DEFB1 and/or DEFB2 is determined.

In certain instances, the presence or level of a particular defensin is detected at the level of mRNA expression with an assay such as, for example, a hybridization assay or an amplification-based assay. In certain other instances, the presence or level of a particular defensin is detected at the level of protein expression using, for example, an immunoassay (e.g., ELISA) or an immunohistochemical assay. Suitable ELISA kits for determining the presence or level of DEFB1 and/or DEFB2 in a sample such as serum, plasma, saliva, urine, or stool are available from, e.g., Alpco Diagnostics (Salem, N.H.), Antigenix America Inc. (Huntington Station, N.Y.), PeproTech (Rocky Hill, N.J.), and/or Alpha Diagnostic Intl. Inc. (San Antonio, Tex.).

β-defensins are antimicrobial peptides implicated in the resistance of epithelial surfaces to microbial colonization. They are the most widely distributed of all defensins, being secreted by leukocytes and epithelial cells of many kinds. For example, they can be found on the tongue, skin, cornea, salivary glands, kidneys, esophagus, and respiratory tract. The human DEFB 1 polypeptide sequence is set forth in, e.g., Genbank Accession No. NP_{—}005209 (SEQ ID NO:13). The human DEFB1 mRNA (coding) sequence is set forth in, e.g., Genbank Accession No. NM_{—}005218 (SEQ ID NO:14). One skilled in the art will appreciate that DEFB1 is also known as BD1, HBD1, DEFB-1, DEFB101, and MGC51822. The human DEFB2 polypeptide sequence is set forth in, e.g., Genbank Accession No. NP_{—}004933 (SEQ ID NO:15). The human DEFB2 mRNA (coding) sequence is set forth in, e.g., Genbank Accession No. NM_{—}004942 (SEQ ID NO:16). One skilled in the art will appreciate that DEFB2 is also known as SAP1, HBD-2, DEFB-2, DEFB102, and DEFB4.

J. Cadherins

The determination of the presence or level of one or more cadherins in a sample is also useful in the present invention. Cadherins are a class of type-1 transmembrane proteins which play important roles in cell adhesion, ensuring that cells within tissues are bound together. They are dependent on calcium (Ca²⁺) ions to function. The cadherin superfamily includes cadherins, protocadherins, desmogleins, and desmocollins, and more. In structure, they share cadherin repeats, which are the extracellular Ca²⁺-binding domains. Cadherins suitable for use in the present invention include, but are not limited to, CDH1-E-cadherin (epithelial), CDH2-N-cadherin (neural), CDH12-cadherin 12, type 2 (N-cadherin 2), CDH3-P-cadherin (placental), CDH4-R-cadherin (retinal), CDH5-VE-cadherin (vascular endothelial), CDH6-K-cadherin (kidney), CDH7-cadherin 7, type 2, CDH8-cadherin 8, type 2, CDH9-cadherin 9, type 2 (T1-cadherin), CDH10-cadherin 10, type 2 (T2-cadherin), CDH11-OB-cadherin (osteoblast), CDH13-T-cadherin-H-cadherin (heart), CDH15-M-cadherin (myotubule), CDH16-KSP-cadherin, CDH17-LI cadherin (liver-intestine), CDH18-cadherin 18, type 2, CDH19-cadherin 19, type 2, CDH20-cadherin 20, type 2, and CDH23-cadherin 23, (neurosensory epithelium). Preferably, the presence or level of E-cadherin is determined.

In certain instances, the presence or level of a particular cadherin is detected at the level of mRNA expression with an assay such as, for example, a hybridization assay or an amplification-based assay. In certain other instances, the presence or level of a particular cadherin is detected at the level of protein expression using, for example, an immunoassay (e.g., ELISA) or an immunohistochemical assay. Suitable ELISA kits for determining the presence or level of E-cadherin in a sample such as serum, plasma, saliva, urine, or stool are available from, e.g., R&D Systems, Inc. (Minneapolis, Minn.) and/or GenWay Biotech, Inc. (San Diego, Calif.).

E-cadherin is a classical cadherin from the cadherin superfamily. It is a calcium dependent cell-cell adhesion glycoprotein comprised of five extracellular cadherin repeats, a transmembrane region, and a highly conserved cytoplasmic tail. The ectodomain of E-cadherin mediates bacterial adhesion to mammalian cells and the cytoplasmic domain is required for internalization. The human E-cadherin polypeptide sequence is set forth in, e.g., Genbank Accession No. NP_{—}004351 (SEQ ID NO:17). The human E-cadherin mRNA (coding) sequence is set forth in, e.g., Genbank Accession No. NM_{—}004360 (SEQ ID NO:18). One skilled in the art will appreciate that E-cadherin is also known as UVO, CDHE, ECAD, LCAM, Arc-1, CD324, and CDH1.

K. Cellular Adhesion Molecules (IgSF CAMs)

The determination of the presence or level of one or more immunoglobulin superfamily cellular adhesion molecules in a sample is also useful in the present invention. As used herein, the term “immunoglobulin superfamily cellular adhesion molecule” (IgSF CAM) includes any of a variety of polypeptides or proteins located on the surface of a cell that have one or more immunoglobulin-like fold domains, and which function in intercellular adhesion and/or signal transduction. In many cases, IgSF CAMs are transmembrane proteins. Non-limiting examples of IgSF CAMs include Neural Cell Adhesion Molecules (NCAMs; e.g., NCAM-120, NCAM-125, NCAM-140, NCAM-145, NCAM-180, NCAM-185, etc.), Intercellular Adhesion Molecules (ICAMs, e.g., ICAM-1, ICAM-2, ICAM-3, ICAM-4, and ICAM-5), Vascular Cell Adhesion Molecule-1 (VCAM-1), Platelet-Endothelial Cell Adhesion Molecule-1 (PECAM-1), L1 Cell Adhesion Molecule (L1CAM), cell adhesion molecule with homology to L1CAM (close homolog of L1) (CHL1), sialic acid binding Ig-like lectins (SIGLECs; e.g., SIGLEC-1, SIGLEC-2, SIGLEC-3, SIGLEC-4, etc.), Nectins (e.g., Nectin-1, Nectin-2, Nectin-3, etc.), and Nectin-like molecules (e.g., Necl-1, Necl-2, Necl-3, Necl-4, and Necl-5). Preferably, the presence or level of ICAM-1 and/or VCAM-1 is determined.

1. Intercellular Adhesion Molecule-1 (ICAM-1)

ICAM-1 is a transmembrane cellular adhesion protein that is continuously present in low concentrations in the membranes of leukocytes and endothelial cells. Upon cytokine stimulation, the concentrations greatly increase. ICAM-1 can be induced by IL-1 and TNFα and is expressed by the vascular endothelium, macrophages, and lymphocytes. In IBD, proinflammatory cytokines cause inflammation by upregulating expression of adhesion molecules such as ICAM-1 and VCAM-1. The increased expression of adhesion molecules recruit more lymphocytes to the infected tissue, resulting in tissue inflammation (see, Goke et al., J., Gastroenterol., 32:480 (1997); and Rijcken et al., Gut, 51:529 (2002)). ICAM-1 is encoded by the intercellular adhesion molecule 1 gene (ICAM1; Entrez GeneID:3383; Genbank Accession No. NM_{—}000201 (SEQ ID NO:19)) and is produced after processing of the intercellular adhesion molecule 1 precursor polypeptide (Genbank Accession No. NP_{—}000192 (SEQ ID NO:20)).

2. Vascular Cell Adhesion Molecule-1 (VCAM-1)

VCAM-1 is a transmembrane cellular adhesion protein that mediates the adhesion of lymphocytes, monocytes, eosinophils, and basophils to vascular endothelium. Upregulation of VCAM-1 in endothelial cells by cytokines occurs as a result of increased gene transcription (e.g., in response to Tumor necrosis factor-alpha (TNFα) and Interleukin-1 (IL-1)). VCAM-1 is encoded by the vascular cell adhesion molecule 1 gene (VCAM1; Entrez GeneID:7412) and is produced after differential splicing of the transcript (Genbank Accession No. NM_{—}001078 (variant 1; SEQ ID NO:21) or NM_{—}080682 (variant 2)), and processing of the precursor polypeptide splice isoform (Genbank Accession No. NP_{—}001069 (isoform a; SEQ ID NO:22) or NP_{—}542413 (isoform b)).

In certain instances, the presence or level of an IgSF CAM is detected at the level of mRNA expression with an assay such as, for example, a hybridization assay or an amplification-based assay. In certain other instances, the presence or level of an IgSF CAM is detected at the level of protein expression using, for example, an immunoassay (e.g., ELISA) or an immunohistochemical assay. Suitable antibodies and/or ELISA kits for determining the presence or level of ICAM-1 and/or VCAM-1 in a sample such as a tissue sample, biopsy, serum, plasma, saliva, urine, or stool are available from, e.g., Invitrogen (Camarillo, Calif.), Santa Cruz Biotechnology, Inc. (Santa Cruz, Calif.), and/or Abcam Inc. (Cambridge, Mass.).

### VI. Methods of Genotyping

A variety of means can be used to genotype an individual at a polymorphic site in the GLI1 gene, MDR1 gene, ATG16L1 gene or any other genetic marker described herein to determine whether a sample (e.g., a nucleic acid sample) contains a specific variant allele or haplotype. For example, enzymatic amplification of nucleic acid from an individual can be conveniently used to obtain nucleic acid for subsequent analysis. The presence or absence of a specific variant allele or haplotype in one or more genetic markers of interest can also be determined directly from the individual's nucleic acid without enzymatic amplification. In certain preferred embodiments, an individual is genotyped at one, two or more of the GLI1, MDR1, and/or ATG16L1 loci.

Genotyping may be used to detect a variety or polymorphisms, including SNPs. In some instances, genotyping assays may be used to detect one or more of the following SNPs: rs2228224 (GLI1); rs2228226 (GLI1); rs2032582 (MDR1); and/or rs2241880 (ATG16L1).

Genotyping of nucleic acid from an individual, whether amplified or not, can be performed using any of various techniques. Useful techniques include, without limitation, polymerase chain reaction (PCR) based analysis assays, sequence analysis assays, and electrophoretic analysis assays, restriction length polymorphism analysis assays, hybridization analysis assays, allele-specific hybridization, oligonucleotide ligation allele-specific elongation/ligation, allele-specific amplification, single-base extension, molecular inversion probe, invasive cleavage, selective termination, restriction length polymorphism, sequencing, single strand conformation polymorphism (SSCP), single strand chain polymorphism, mismatch-cleaving, and denaturing gradient gel electrophoresis, all of which can be used alone or in combination. As used herein, the term “nucleic acid” includes a polynucleotide such as a single- or double-stranded DNA or RNA molecule including, for example, genomic DNA, cDNA and mRNA. This term encompasses nucleic acid molecules of both natural and synthetic origin as well as molecules of linear, circular, or branched configuration representing either the sense or antisense strand, or both, of a native nucleic acid molecule. It is understood that such nucleic acids can be unpurified, purified, or attached, for example, to a synthetic material such as a bead or column matrix.

Material containing nucleic acid is routinely obtained from individuals. Such material is any biological matter from which nucleic acid can be prepared. As non-limiting examples, material can be whole blood, serum, plasma, saliva, cheek swab, sputum, or other bodily fluid or tissue that contains nucleic acid. In one embodiment, a method of the present invention is practiced with whole blood, which can be obtained readily by non-invasive means and used to prepare genomic DNA. In another embodiment, genotyping involves amplification of an individual's nucleic acid using the polymerase chain reaction (PCR). Use of PCR for the amplification of nucleic acids is well known in the art (see, e.g., Mullis et al. (Eds.), The Polymerase Chain Reaction, Birkhäuser, Boston, (1994)). In yet another embodiment, PCR amplification is performed using one or more fluorescently labeled primers. In a further embodiment, PCR amplification is performed using one or more labeled or unlabeled primers that contain a DNA minor groove binder.

Any of a variety of different primers can be used to amplify an individual's nucleic acid by PCR in order to determine the presence or absence of a variant allele in the GLI1 gene, MDR1 gene or ATG16L1 gene or other genetic marker in a method of the invention. As understood by one skilled in the art, primers for PCR analysis can be designed based on the sequence flanking the polymorphic site(s) of interest in the GLI1 gene, MDR1 gene or ATG16L1 gene or other genetic marker. As a non-limiting example, a sequence primer can contain from about 15 to about 30 nucleotides of a sequence upstream or downstream of the polymorphic site of interest in the GLI1 gene, MDR1 gene or ATG16L1 gene or other genetic marker. Such primers generally are designed to have sufficient guanine and cytosine content to attain a high melting temperature which allows for a stable annealing step in the amplification reaction. Several computer programs, such as Primer Select, are available to aid in the design of PCR primers.

A Taqman® allelic discrimination assay available from Applied Biosystems can be useful for genotyping an individual at a polymorphic site and thereby determining the presence or absence of a particular variant allele or haplotype in the GLI1 gene, MDR1 gene or ATG16L1 gene or other genetic marker described herein. In a Taqman® allelic discrimination assay, a specific fluorescent dye-labeled probe for each allele is constructed. The probes contain different fluorescent reporter dyes such as FAM and VIC™ to differentiate amplification of each allele. In addition, each probe has a quencher dye at one end which quenches fluorescence by fluorescence resonance energy transfer. During PCR, each probe anneals specifically to complementary sequences in the nucleic acid from the individual. The 5′ nuclease activity of Taq polymerase is used to cleave only probe that hybridizes to the allele. Cleavage separates the reporter dye from the quencher dye, resulting in increased fluorescence by the reporter dye. Thus, the fluorescence signal generated by PCR amplification indicates which alleles are present in the sample. Mismatches between a probe and allele reduce the efficiency of both probe hybridization and cleavage by Taq polymerase, resulting in little to no fluorescent signal. Those skilled in the art understand that improved specificity in allelic discrimination assays can be achieved by conjugating a DNA minor groove binder (MGB) group to a DNA probe as described, e.g., in Kutyavin et al., Nuc. Acids Research 28:655-661 (2000). Minor groove binders include, but are not limited to, compounds such as dihydrocyclopyrroloindole tripeptide (DPI3).

Sequence analysis can also be useful for genotyping an individual according to the methods described herein to determine the presence or absence of a particular variant allele or haplotype in the GLI1 gene, MDR1 gene or ATG16L1 gene or other genetic marker. As is known by those skilled in the art, a variant allele of interest can be detected by sequence analysis using the appropriate primers, which are designed based on the sequence flanking the polymorphic site of interest in the GLI1 gene, MDR1 gene or ATG16L1 gene or other genetic marker. For example, a GLI1 gene, MDR1 gene or ATG16L1 variant allele can be detected by sequence analysis using primers designed by one of skill in the art. Additional or alternative sequence primers can contain from about 15 to about 30 nucleotides of a sequence that corresponds to a sequence about 40 to about 400 base pairs upstream or downstream of the polymorphic site of interest in the GLI1 gene, MDR1 gene or ATG16L1 gene or other genetic marker. Such primers are generally designed to have sufficient guanine and cytosine content to attain a high melting temperature which allows for a stable annealing step in the sequencing reaction.

The term “sequence analysis” includes any manual or automated process by which the order of nucleotides in a nucleic acid is determined. As an example, sequence analysis can be used to determine the nucleotide sequence of a sample of DNA. The term sequence analysis encompasses, without limitation, chemical and enzymatic methods such as dideoxy enzymatic methods including, for example, Maxam-Gilbert and Sanger sequencing as well as variations thereof. The term sequence analysis further encompasses, but is not limited to, capillary array DNA sequencing, which relies on capillary electrophoresis and laser-induced fluorescence detection and can be performed using instruments such as the MegaBACE 1000 or ABI 3700. As additional non-limiting examples, the term sequence analysis encompasses thermal cycle sequencing (see, Sears et al., Biotechniques 13:626-633 (1992)); solid-phase sequencing (see, Zimmerman et al., Methods Mol. Cell Biol. 3:39-42 (1992); and sequencing with mass spectrometry, such as matrix-assisted laser desorption/ionization time-of-flight mass spectrometry (see, MALDI-TOF MS; Fu et al., Nature Biotech. 16:381-384 (1998)). The term sequence analysis further includes, but is not limited to, sequencing by hybridization (SBH), which relies on an array of all possible short oligonucleotides to identify a segment of sequence (see, Chee et al., Science 274:610-614 (1996); Drmanac et al., Science 260:1649-1652 (1993); and Drmanac et al., Nature Biotech. 16:54-58 (1998)). One skilled in the art understands that these and additional variations are encompassed by the term sequence analysis as defined herein.

Electrophoretic analysis also can be useful in genotyping an individual according to the methods of the present invention to determine the presence or absence of a particular variant allele or haplotype in the GLI1 gene, MDR1 gene or ATG16L1 gene or other genetic marker. “Electrophoretic analysis” as used herein in reference to one or more nucleic acids such as amplified fragments includes a process whereby charged molecules are moved through a stationary medium under the influence of an electric field. Electrophoretic migration separates nucleic acids primarily on the basis of their charge, which is in proportion to their size, with smaller molecules migrating more quickly. The term electrophoretic analysis includes, without limitation, analysis using slab gel electrophoresis, such as agarose or polyacrylamide gel electrophoresis, or capillary electrophoresis. Capillary electrophoretic analysis generally occurs inside a small-diameter (50-100 m) quartz capillary in the presence of high (kilovolt-level) separating voltages with separation times of a few minutes. Using capillary electrophoretic analysis, nucleic acids are conveniently detected by UV absorption or fluorescent labeling, and single-base resolution can be obtained on fragments up to several hundred base pairs. Such methods of electrophoretic analysis, and variations thereof, are well known in the art, as described, for example, in Ausubel et al., Current Protocols in Molecular Biology Chapter 2 (Supplement 45) John Wiley & Sons, Inc. New York (1999).

Restriction fragment length polymorphism (RFLP) analysis can also be useful for genotyping an individual according to the methods of the present invention to determine the presence or absence of a particular variant allele or haplotype in the GLI1 gene, MDR1 gene or ATG16L1 gene or other genetic marker (see, Jarcho et al. in Dracopoli et al., Current Protocols in Human Genetics pages 2.7.1-2.7.5, John Wiley & Sons, New York; Innis et al., (Ed.), PCR Protocols, San Diego: Academic Press, Inc. (1990)). As used herein, “restriction fragment length polymorphism analysis” includes any method for distinguishing polymorphic alleles using a restriction enzyme, which is an endonuclease that catalyzes degradation of nucleic acid following recognition of a specific base sequence, generally a palindrome or inverted repeat. One skilled in the art understands that the use of RFLP analysis depends upon an enzyme that can differentiate a variant allele from a wild-type or other allele at a polymorphic site.

In addition, allele-specific oligonucleotide hybridization can be useful for genotyping an individual in the methods described herein to determine the presence or absence of a particular variant allele or haplotype in the GLI1 gene, MDR1 gene or ATG16L1 gene or other genetic marker. Allele-specific oligonucleotide hybridization is based on the use of a labeled oligonucleotide probe having a sequence perfectly complementary, for example, to the sequence encompassing the variant allele. Under appropriate conditions, the variant allele-specific probe hybridizes to a nucleic acid containing the variant allele but does not hybridize to the one or more other alleles, which have one or more nucleotide mismatches as compared to the probe. If desired, a second allele-specific oligonucleotide probe that matches an alternate (e.g., wild-type) allele can also be used. Similarly, the technique of allele-specific oligonucleotide amplification can be used to selectively amplify, for example, a variant allele by using an allele-specific oligonucleotide primer that is perfectly complementary to the nucleotide sequence of the variant allele but which has one or more mismatches as compared to other alleles (Mullis et al., supra). One skilled in the art understands that the one or more nucleotide mismatches that distinguish between the variant allele and other alleles are often located in the center of an allele-specific oligonucleotide primer to be used in the allele-specific oligonucleotide hybridization. In contrast, an allele-specific oligonucleotide primer to be used in PCR amplification generally contains the one or more nucleotide mismatches that distinguish between the variant and other alleles at the 3′ end of the primer.

A heteroduplex mobility assay (HMA) is another well-known assay that can be used for genotyping in the methods of the present invention to determine the presence or absence of a particular variant allele or haplotype in the GLI1 gene, MDR1 gene or ATG16L1 gene or other genetic marker. HMA is useful for detecting the presence of a variant allele since a DNA duplex carrying a mismatch has reduced mobility in a polyacrylamide gel compared to the mobility of a perfectly base-paired duplex (see, Delwart et al., Science, 262:1257-1261 (1993); White et al., Genomics, 12:301-306 (1992)).

The technique of single strand conformational polymorphism (SSCP) can also be useful for genotyping in the methods described herein to determine the presence or absence of a particular variant allele or haplotype in the GLI1 gene, MDR1 gene or ATG16L1 gene or other genetic marker (see, Hayashi, Methods Applic., 1:34-38 (1991)). This technique is used to detect variant alleles based on differences in the secondary structure of single-stranded DNA that produce an altered electrophoretic mobility upon non-denaturing gel electrophoresis. Variant alleles are detected by comparison of the electrophoretic pattern of the test fragment to corresponding standard fragments containing known alleles.

Denaturing gradient gel electrophoresis (DGGE) can also be useful in the methods of the invention to determine the presence or absence of a particular variant allele or haplotype in the GLI1 gene, MDR1 gene or ATG16L1 gene or other genetic marker. In DGGE, double-stranded DNA is electrophoresed in a gel containing an increasing concentration of denaturant; double-stranded fragments made up of mismatched alleles have segments that melt more rapidly, causing such fragments to migrate differently as compared to perfectly complementary sequences (see, Sheffield et al., “Identifying DNA Polymorphisms by Denaturing Gradient Gel Electrophoresis” in Innis et al., supra, 1990).

Other molecular methods useful for genotyping an individual are known in the art and useful in the methods of the present invention. Such well-known genotyping approaches include, without limitation, automated sequencing and RNase mismatch techniques (see, Winter et al., Proc. Natl. Acad. Sci., 82:7575-7579 (1985)). Furthermore, one skilled in the art understands that, where the presence or absence of multiple variant alleles is to be determined, individual variant alleles can be detected by any combination of molecular methods. See, in general, Birren et al. (Eds.) Genome Analysis: A Laboratory Manual Volume 1 (Analyzing DNA) New York, Cold Spring Harbor Laboratory Press (1997). In addition, one skilled in the art understands that multiple variant alleles can be detected in individual reactions or in a single reaction (a “multiplex” assay).

In view of the above, one skilled in the art realizes that the methods of the present invention for diagnosing IBD, diagnosing UC, or differentiating between UC and CD (e.g., by determining the presence or absence of one or more GLI1, MDR1, or ATG16L1 variant alleles) can be practiced using one or any combination of the well-known genotyping assays described above or other assays known in the art.

### VII. Assays

Any of a variety of assays, techniques, and kits known in the art can be used to detect or determine the presence (or absence) or level (e.g., concentration) of one or more biochemical, serological, or protein markers in a sample to diagnose IBD, to classify the diagnosis of IBD (e.g., CD or UC), or to differentiate between UC and CD.

Flow cytometry can be used to detect the presence or level of one or more markers in a sample. Such flow cytometric assays, including bead based immunoassays, can be used to determine, e.g., antibody marker levels in the same manner as described for detecting serum antibodies to Candida albicans and HIV proteins (see, e.g., Bishop and Davis, J. Immunol. Methods, 210:79-87 (1997); McHugh et al., J. Immunol. Methods, 116:213 (1989); Scillian et al., Blood, 73:2041 (1989)).

Phage display technology for expressing a recombinant antigen specific for a marker can also be used to detect the presence or level of one or more markers in a sample. Phage particles expressing an antigen specific for, e.g., an antibody marker can be anchored, if desired, to a multi-well plate using an antibody such as an anti-phage monoclonal antibody (Felici et al., “Phage-Displayed Peptides as Tools for Characterization of Human Sera” in Abelson (Ed.), Methods in Enzymol., 267, San Diego: Academic Press, Inc. (1996)).

A variety of immunoassay techniques, including competitive and non-competitive immunoassays, can be used to detect the presence or level of one or more markers in a sample (see, e.g., Self and Cook, Curr. Opin. Biotechnol., 7:60-65 (1996)). The term immunoassay encompasses techniques including, without limitation, enzyme immunoassays (EIA) such as enzyme multiplied immunoassay technique (EMIT), enzyme-linked immunosorbent assay (ELISA), antigen capture ELISA, sandwich ELISA, IgM antibody capture ELISA (MAC ELISA), and microparticle enzyme immunoassay (MEIA); capillary electrophoresis immunoassays (CEIA); radioimmunoassays (RIA); immunoradiometric assays (IRMA); fluorescence polarization immunoassays (FPIA); and chemiluminescence assays (CL). If desired, such immunoassays can be automated. Immunoassays can also be used in conjunction with laser induced fluorescence (see, e.g., Schmalzing and Nashabeh, Electrophoresis, 18:2184-2193 (1997); Bao, J. Chromatogr. B. Biomed. Sci., 699:463-480 (1997)). Liposome immunoassays, such as flow-injection liposome immunoassays and liposome immunosensors, are also suitable for use in the present invention (see, e.g., Rongen et al., J. Immunol. Methods, 204:105-133 (1997)). In addition, nephelometry assays, in which the formation of protein/antibody complexes results in increased light scatter that is converted to a peak rate signal as a function of the marker concentration, are suitable for use in the present invention. Nephelometry assays are commercially available from Beckman Coulter (Brea, Calif.; Kit #449430) and can be performed using a Behring Nephelometer Analyzer (Fink et al., J. Clin. Chem. Clin. Biol. Chem., 27:261-276 (1989)).

Antigen capture ELISA can be useful for detecting the presence or level of one or more markers in a sample. For example, in an antigen capture ELISA, an antibody directed to a marker of interest is bound to a solid phase and sample is added such that the marker is bound by the antibody. After unbound proteins are removed by washing, the amount of bound marker can be quantitated using, e.g., a radioimmunoassay (see, e.g., Harlow and Lane, Antibodies: A Laboratory Manual, Cold Spring Harbor Laboratory, New York, 1988)). Sandwich ELISA can also be suitable for use in the present invention. For example, in a two-antibody sandwich assay, a first antibody is bound to a solid support, and the marker of interest is allowed to bind to the first antibody. The amount of the marker is quantitated by measuring the amount of a second antibody that binds the marker. The antibodies can be immobilized onto a variety of solid supports, such as magnetic or chromatographic matrix particles, the surface of an assay plate (e.g., microtiter wells), pieces of a solid substrate material or membrane (e.g., plastic, nylon, paper), and the like. An assay strip can be prepared by coating the antibody or a plurality of antibodies in an array on a solid support. This strip can then be dipped into the test sample and processed quickly through washes and detection steps to generate a measurable signal, such as a colored spot.

A radioimmunoassay using, for example, an iodine-125 (¹²⁵I) labeled secondary antibody (Harlow and Lane, supra) is also suitable for detecting the presence or level of one or more markers in a sample. A secondary antibody labeled with a chemiluminescent marker can also be suitable for use in the present invention. A chemiluminescence assay using a chemiluminescent secondary antibody is suitable for sensitive, non-radioactive detection of marker levels. Such secondary antibodies can be obtained commercially from various sources, e.g., Amersham Lifesciences, Inc. (Arlington Heights, Ill.).

The immunoassays described above are particularly useful for detecting the presence (or absence) or level of one or more serological markers in a sample. As a non-limiting example, a fixed neutrophil ELISA is useful for determining whether a sample is positive for ANCA or for determining ANCA levels in a sample. Similarly, an ELISA using yeast cell wall phosphopeptidomannan is useful for determining whether a sample is positive for ASCA-IgA and/or ASCA-IgG, or for determining ASCA-IgA and/or ASCA-IgG levels in a sample. An ELISA using OmpC protein or a fragment thereof is useful for determining whether a sample is positive for anti-OmpC antibodies, or for determining anti-OmpC antibody levels in a sample. An ELISA using I2 protein or a fragment thereof is useful for determining whether a sample is positive for anti-I2 antibodies, or for determining anti-I2 antibody levels in a sample. An ELISA using flagellin protein (e.g., Cbir-1 flagellin) or a fragment thereof is useful for determining whether a sample is positive for anti-flagellin antibodies, or for determining anti-flagellin antibody levels in a sample. In addition, the immunoassays described above are particularly useful for detecting the presence or level of other serological markers in a sample.

Specific immunological binding of the antibody to the marker of interest can be detected directly or indirectly. Direct labels include fluorescent or luminescent tags, metals, dyes, radionuclides, and the like, attached to the antibody. An antibody labeled with iodine-125 (¹²⁵I) can be used for determining the levels of one or more markers in a sample. A chemiluminescence assay using a chemiluminescent antibody specific for the marker is suitable for sensitive, non-radioactive detection of marker levels. An antibody labeled with fluorochrome is also suitable for determining the levels of one or more markers in a sample. Examples of fluorochromes include, without limitation, DAPI, fluorescein, Hoechst 33258, R-phycocyanin, B-phycoerythrin, R-phycoerythrin, rhodamine, Texas red, and lissamine. Secondary antibodies linked to fluorochromes can be obtained commercially, e.g., goat F(ab′)₂ anti-human IgG-FITC is available from Tago Immunologicals (Burlingame, Calif.).

Indirect labels include various enzymes well-known in the art, such as horseradish peroxidase (HRP), alkaline phosphatase (AP), β-galactosidase, urease, and the like. A horseradish-peroxidase detection system can be used, for example, with the chromogenic substrate tetramethylbenzidine (TMB), which yields a soluble product in the presence of hydrogen peroxide that is detectable at 450 nm. An alkaline phosphatase detection system can be used with the chromogenic substrate p-nitrophenyl phosphate, for example, which yields a soluble product readily detectable at 405 nm. Similarly, a β-galactosidase detection system can be used with the chromogenic substrate o-nitrophenyl-β-D-galactopyranoside (ONPG), which yields a soluble product detectable at 410 nm. An urease detection system can be used with a substrate such as urea-bromocresol purple (Sigma Immunochemicals; St. Louis, Mo.). A useful secondary antibody linked to an enzyme can be obtained from a number of commercial sources, e.g., goat F(ab′)₂ anti-human IgG-alkaline phosphatase can be purchased from Jackson ImmunoResearch (West Grove, Pa.).

A signal from the direct or indirect label can be analyzed, for example, using a spectrophotometer to detect color from a chromogenic substrate; a radiation counter to detect radiation such as a gamma counter for detection of ¹²⁵I; or a fluorometer to detect fluorescence in the presence of light of a certain wavelength. For detection of enzyme-linked antibodies, a quantitative analysis of the amount of marker levels can be made using a spectrophotometer such as an EMAX Microplate Reader (Molecular Devices; Menlo Park, Calif.) in accordance with the manufacturer's instructions. If desired, the assays described herein can be automated or performed robotically, and the signal from multiple samples can be detected simultaneously.

Quantitative Western blotting can also be used to detect or determine the presence or level of one or more markers in a sample. Western blots can be quantitated by well-known methods such as scanning densitometry or phosphorimaging. As a non-limiting example, protein samples are electrophoresed on 10% SDS-PAGE Laemmli gels. Primary murine monoclonal antibodies are reacted with the blot, and antibody binding can be confirmed to be linear using a preliminary slot blot experiment. Goat anti-mouse horseradish peroxidase-coupled antibodies (BioRad) are used as the secondary antibody, and signal detection performed using chemiluminescence, for example, with the Renaissance chemiluminescence kit (New England Nuclear; Boston, Mass.) according to the manufacturer's instructions. Autoradiographs of the blots are analyzed using a scanning densitometer (Molecular Dynamics; Sunnyvale, Calif.) and normalized to a positive control. Values are reported, for example, as a ratio between the actual value to the positive control (densitometric index). Such methods are well known in the art as described, for example, in Parra et al., J. Vasc. Surg., 28:669-675 (1998).

Alternatively, a variety of immunohistochemical assay techniques can be used to detect or determine the presence or level of one or more markers in a sample. The term “immunohistochemical assay” encompasses techniques that utilize the visual detection of fluorescent dyes or enzymes coupled (i.e., conjugated) to antibodies that react with the marker of interest using fluorescent microscopy or light microscopy and includes, without limitation, direct fluorescent antibody assay, indirect fluorescent antibody (IFA) assay, anticomplement immunofluorescence, avidin-biotin immunofluorescence, and immunoperoxidase assays. An IFA assay, for example, is useful for determining whether a sample is positive for ANCA, the level of ANCA in a sample, whether a sample is positive for pANCA, the level of pANCA in a sample, and/or an ANCA staining pattern (e.g., cANCA, pANCA, NSNA, and/or SAPPA staining pattern). The concentration of ANCA in a sample can be quantitated, e.g., through endpoint titration or through measuring the visual intensity of fluorescence compared to a known reference standard.

In certain other embodiments, the presence or level of a marker of interest can be determined by detecting or quantifying the amount of the purified marker. Purification of the marker can be achieved, for example, by high pressure liquid chromatography (HPLC), alone or in combination with mass spectrometry (e.g., MALDI/MS, MALDI-TOF/MS, SELDI-TOF/MS, tandem MS, etc.). Qualitative or quantitative detection of a marker of interest can also be determined by well-known methods including, without limitation, Bradford assays, Coomassie blue staining, silver staining, assays for radiolabeled protein, and mass spectrometry.

In some aspects, the analysis of a plurality of markers may be carried out separately or simultaneously with one test sample. For separate or sequential assay of markers, suitable apparatuses include clinical laboratory analyzers such as the ElecSys (Roche), the AxSym (Abbott), the Access (Beckman), the ADVIA®, the CENTAUR® (Bayer), and the NICHOLS ADVANTAGE® (Nichols Institute) immunoassay systems. Preferred apparatuses or protein chips perform simultaneous assays of a plurality of markers on a single surface. Particularly useful physical formats comprise surfaces having a plurality of discrete, addressable locations for the detection of a plurality of different markers. Such formats include, e.g., protein microarrays, or “protein chips” (see, e.g., Ng et al., J. Cell Mol. Med., 6:329-340 (2002)) and certain capillary devices (see, e.g., U.S. Pat. No. 6,019,944). In these embodiments, each discrete surface location may comprise antibodies to immobilize one or more markers for detection at each location. Surfaces may alternatively comprise one or more discrete particles (e.g., microparticles or nanoparticles) immobilized at discrete locations of a surface, where the microparticles comprise antibodies to immobilize one or more markers for detection.

In addition to the above-described assays for detecting the presence or level of various markers of interest, analysis of marker mRNA levels using routine techniques such as Northern analysis, reverse-transcriptase polymerase chain reaction (RT-PCR), or any other methods based on hybridization to a nucleic acid sequence that is complementary to a portion of the marker coding sequence (e.g., slot blot hybridization) are also within the scope of the present invention. Applicable PCR amplification techniques are described in, e.g., Ausubel et al., Current Protocols in Molecular Biology, John Wiley & Sons, Inc. New York (1984-2008), Chapter 7 and Supplement 47; Theophilus et al., “PCR Mutation Detection Protocols,” Humana Press, (2002); Innis et al., PCR Protocols, San Diego, Academic Press, Inc. (1990); and Maniatis, et al., Molecular Cloning: A Laboratory Manual, Cold Spring Harbor Lab., New York, (1982). General nucleic acid hybridization methods are described in Anderson, “Nucleic Acid Hybridization,” BIOS Scientific Publishers, (1999). Amplification or hybridization of a plurality of transcribed nucleic acid sequences (e.g., mRNA or cDNA) can also be performed from mRNA or cDNA sequences arranged in a microarray. Microarray methods are generally described in Hardiman, “Microarrays Methods and Applications: Nuts & Bolts,” DNA Press, (2003); and Baldi et al., “DNA Microarrays and Gene Expression: From Experiments to Data Analysis and Modeling,” Cambridge University Press, (2002).

Several markers of interest may be combined into one test for efficient processing of a multiple of samples. In addition, one skilled in the art would recognize the value of testing multiple samples (e.g., at successive time points, etc.) from the same subject. Such testing of serial samples can allow the identification of changes in marker levels over time. Increases or decreases in marker levels, as well as the absence of change in marker levels, can also provide useful prognostic and predictive information to facilitate in the diagnosis of UC or the differentiation between UC and CD.

In view of the above, one skilled in the art realizes that the methods of the invention for providing diagnostic information regarding IBD, and most specifically diagnosing UC, or for differentiating between UC and CD, can be practiced using one or any combination of the well-known assays described above or other assays known in the art.

### VIII. Statistical Analysis

In some aspects, the present invention provides methods and systems for diagnosing IBD, for classifying the diagnosis of IBD (e.g., CD or UC), for classifying the subtype of IBD as UC or for differentiating between UC and CD. In particular embodiments, quantile analysis is applied to the presence, level, and/or genotype of one or more IBD markers determined by any of the assays described herein to diagnose IBD, diagnose UC, or differentiate between UC and CD. In other embodiments, one or more learning statistical classifier systems are applied to the presence, level, and/or genotype of one or more IBD markers determined by any of the assays described herein to diagnose IBD, diagnose UC, or differentiate between UC and CD. As described herein, the statistical analyses of the present invention advantageously provide improved sensitivity, specificity, negative predictive value, positive predictive value, and/or overall accuracy for diagnosing IBD, diagnosing UC, and differentiating between UC and CD.

The term “statistical analysis” or “statistical algorithm” or “statistical process” includes any of a variety of statistical methods and models used to determine relationships between variables. In the present invention, the variables are the presence, level, or genotype of at least one marker of interest. Any number of markers can be analyzed using a statistical analysis described herein. For example, the presence or level of 1, 2, 3, 4, 5, 6, 7, 8, 9, 10, 11, 12, 13, 14, 15, 16, 17, 18, 19, 20, 25, 30, 35, 40, 45, 50, 55, 60, or more markers can be included in a statistical analysis. In one embodiment, logistic regression is used. In another embodiment, linear regression is used. In certain preferred embodiments, the statistical analyses of the present invention comprise a quantile measurement of one or more markers, e.g., within a given population, as a variable. Quantiles are a set of “cut points” that divide a sample of data into groups containing (as far as possible) equal numbers of observations. For example, quartiles are values that divide a sample of data into four groups containing (as far as possible) equal numbers of observations. The lower quartile is the data value a quarter way up through the ordered data set; the upper quartile is the data value a quarter way down through the ordered data set. Quintiles are values that divide a sample of data into five groups containing (as far as possible) equal numbers of observations. The present invention can also include the use of percentile ranges of marker levels (e.g., tertiles, quartile, quintiles, etc.), or their cumulative indices (e.g., quartile sums of marker levels to obtain quartile sum scores (QSS), etc.) as variables in the statistical analyses (just as with continuous variables).

In preferred embodiments, the present invention involves detecting or determining the presence, level (e.g., magnitude), and/or genotype of one or more markers of interest using quartile analysis. In this type of statistical analysis, the level of a marker of interest is defined as being in the first quartile (<25%), second quartile (25-50%), third quartile (51%-<75%), or fourth quartile (75-100%) in relation to a reference database of samples. These quartiles may be assigned a quartile score of 1, 2, 3, and 4, respectively. In certain instances, a marker that is not detected in a sample is assigned a quartile score of 0 or 1, while a marker that is detected (e.g., present) in a sample (e.g., sample is positive for the marker) is assigned a quartile score of 4. In some embodiments, quartile 1 represents samples with the lowest marker levels, while quartile 4 represent samples with the highest marker levels. In other embodiments, quartile 1 represents samples with a particular marker genotype (e.g., wild-type allele), while quartile 4 represent samples with another particular marker genotype (e.g., allelic variant). The reference database of samples can include a large spectrum of IBD (e.g., CD and/or UC) patients. From such a database, quartile cut-offs can be established. A non-limiting example of quartile analysis suitable for use in the present invention is described in, e.g., Mow et al., Gastroenterology, 126:414-24 (2004).

In some embodiments, the statistical analyses of the present invention comprise one or more learning statistical classifier systems. As used herein, the term “learning statistical classifier system” includes a machine learning algorithmic technique capable of adapting to complex data sets (e.g., panel of markers of interest) and making decisions based upon such data sets. In some embodiments, a single learning statistical classifier system such as a decision/classification tree (e.g., random forest (RF) or classification and regression tree (C&RT)) is used. In other embodiments, a combination of 2, 3, 4, 5, 6, 7, 8, 9, 10, or more learning statistical classifier systems are used, preferably in tandem. Examples of learning statistical classifier systems include, but are not limited to, those using inductive learning (e.g., decision/classification trees such as random forests, classification and regression trees (C&RT), boosted trees, etc.), Probably Approximately Correct (PAC) learning, connectionist learning (e.g., neural networks (NN), artificial neural networks (ANN), neuro fuzzy networks (NFN), network structures, perceptrons such as multi-layer perceptrons, multi-layer feed-forward networks, applications of neural networks, Bayesian learning in belief networks, etc.), reinforcement learning (e.g., passive learning in a known environment such as naïve learning, adaptive dynamic learning, and temporal difference learning, passive learning in an unknown environment, active learning in an unknown environment, learning action-value functions, applications of reinforcement learning, etc.), and genetic algorithms and evolutionary programming. Other learning statistical classifier systems include support vector machines (e.g., Kernel methods), multivariate adaptive regression splines (MARS), Levenberg-Marquardt algorithms, Gauss-Newton algorithms, mixtures of Gaussians, gradient descent algorithms, and learning vector quantization (LVQ).

Random forests are learning statistical classifier systems that are constructed using an algorithm developed by Leo Breiman and Adele Cutler. Random forests use a large number of individual decision trees and decide the class by choosing the mode (i.e., most frequently occurring) of the classes as determined by the individual trees. Random forest analysis can be performed, e.g., using the RandomForests software available from Salford Systems (San Diego, Calif.). See, e.g., Breiman, Machine Learning, 45:5-32 (2001); and http://stat-www.berkeley.edu/users/breiman/RandomForests/cc_home.htm, for a description of random forests.

Classification and regression trees represent a computer intensive alternative to fitting classical regression models and are typically used to determine the best possible model for a categorical or continuous response of interest based upon one or more predictors. Classification and regression tree analysis can be performed, e.g., using the C&RT software available from Salford Systems or the Statistica data analysis software available from StatSoft, Inc. (Tulsa, Okla.). A description of classification and regression trees is found, e.g., in Breiman et al. “Classification and Regression Trees,” Chapman and Hall, New York (1984); and Steinberg et al., “CART: Tree-Structured Non-Parametric Data Analysis,” Salford Systems, San Diego, (1995).

Neural networks are interconnected groups of artificial neurons that use a mathematical or computational model for information processing based on a connectionist approach to computation. Typically, neural networks are adaptive systems that change their structure based on external or internal information that flows through the network. Specific examples of neural networks include feed-forward neural networks such as perceptrons, single-layer perceptrons, multi-layer perceptrons, backpropagation networks, ADALINE networks, MADALINE networks, Learnmatrix networks, radial basis function (RBF) networks, and self-organizing maps or Kohonen self-organizing networks; recurrent neural networks such as simple recurrent networks and Hopfield networks; stochastic neural networks such as Boltzmann machines; modular neural networks such as committee of machines and associative neural networks; and other types of networks such as instantaneously trained neural networks, spiking neural networks, dynamic neural networks, and cascading neural networks. Neural network analysis can be performed, e.g., using the Statistica data analysis software available from StatSoft, Inc. See, e.g., Freeman et al., In “Neural Networks: Algorithms, Applications and Programming Techniques,” Addison-Wesley Publishing Company (1991); Zadeh, Information and Control, 8:338-353 (1965); Zadeh, “IEEE Trans. on Systems, Man and Cybernetics,” 3:28-44 (1973); Gersho et al., In “Vector Quantization and Signal Compression,” Kluywer Academic Publishers, Boston, Dordrecht, London (1992); and Hassoun, “Fundamentals of Artificial Neural Networks,” MIT Press, Cambridge, Mass., London (1995), for a description of neural networks.

Support vector machines are a set of related supervised learning techniques used for classification and regression and are described, e.g., in Cristianini et al., “An Introduction to Support Vector Machines and Other Kernel-Based Learning Methods,” Cambridge University Press (2000). Support vector machine analysis can be performed, e.g., using the SVM^{1ight} software developed by Thorsten Joachims (Cornell University) or using the LIBSVM software developed by Chih-Chung Chang and Chih-Jen Lin (National Taiwan University).

The various statistical methods and models described herein can be trained and tested using a cohort of samples (e.g., serological and/or genomic samples) from healthy individuals and IBD (e.g., CD and/or UC) patients. For example, samples from patients diagnosed by a physician, and preferably by a gastroenterologist, as having IBD or a clinical subtype thereof using a biopsy, colonoscopy, or an immunoassay as described in, e.g., U.S. Pat. No. 6,218,129, are suitable for use in training and testing the statistical methods and models of the present invention. Samples from patients diagnosed with IBD can also be stratified into Crohn's disease or ulcerative colitis using an immunoassay as described in, e.g., U.S. Pat. Nos. 5,750,355 and 5,830,675. Samples from healthy individuals can include those that were not identified as IBD samples. One skilled in the art will know of additional techniques and diagnostic criteria for obtaining a cohort of patient samples that can be used in training and testing the statistical methods and models of the present invention.

As used herein, the term “sensitivity” refers to the probability that a diagnostic, prognostic, or predictive method of the present invention gives a positive result when the sample is positive, e.g., having the predicted diagnosis of IBD, the predicted diagnosis of UC, or the predicted differentiation between the UC and CD subtypes of IBD. Sensitivity is calculated as the number of true positive results divided by the sum of the true positives and false negatives. Sensitivity essentially is a measure of how well the present invention correctly identifies those who have the predicted diagnosis of IBD, the predicted diagnosis of UC, or the predicted differentiation between the UC and CD subtypes of IBD from those who do not have the predicted diagnosis of IBD, the predicted diagnosis of UC, or the predicted differentiation between the UC and CD subtypes of IBD. The statistical methods and models can be selected such that the sensitivity is at least about 60%, and can be, e.g., at least about 65%, 70%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%.

The term “specificity” refers to the probability that a diagnostic, prognostic, or predictive method of the present invention gives a negative result when the sample is not positive, e.g., not having the predicted diagnosis of IBD, the predicted diagnosis of UC, or the predicted differentiation between the UC and CD subtypes of IBD. Specificity is calculated as the number of true negative results divided by the sum of the true negatives and false positives. Specificity essentially is a measure of how well the present invention excludes those who do not have the predicted diagnosis of IBD, the predicted diagnosis of UC, or the predicted differentiation between the UC and CD subtypes of IBD from those who do have the predicted diagnosis of IBD, the predicted diagnosis of UC, or the predicted differentiation between the UC and CD subtypes of IBD. The statistical methods and models can be selected such that the specificity is at least about 60%, and can be, e.g., at least about 65%, 70%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%.

As used herein, the term “negative predictive value” or “NPV” refers to the probability that an individual identified as not having the predicted diagnosis of IBD, the predicted diagnosis of UC, or the predicted differentiation between the UC and CD subtypes of IBD actually does not have the predicted diagnosis of IBD, the predicted diagnosis of UC, or the predicted differentiation between the UC and CD subtypes of IBD. Negative predictive value can be calculated as the number of true negatives divided by the sum of the true negatives and false negatives. Negative predictive value is determined by the characteristics of the diagnostic or prognostic method as well as the prevalence of the disease in the population analyzed. The statistical methods and models can be selected such that the negative predictive value in a population having a disease prevalence is in the range of about 70% to about 99% and can be, for example, at least about 70%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%.

The term “positive predictive value” or “PPV” refers to the probability that an individual identified as having the predicted diagnosis of IBD, the predicted diagnosis of UC, or the predicted differentiation between the UC and CD subtypes of IBD actually has the predicted diagnosis of IBD, the predicted diagnosis of UC, or the predicted differentiation between the UC and CD subtypes of IBD. Positive predictive value can be calculated as the number of true positives divided by the sum of the true positives and false positives. Positive predictive value is determined by the characteristics of the diagnostic or prognostic method as well as the prevalence of the disease in the population analyzed. The statistical methods and models can be selected such that the positive predictive value in a population having a disease prevalence is in the range of about 70% to about 99% and can be, for example, at least about 70%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%.

Predictive values, including negative and positive predictive values, are influenced by the prevalence of the disease in the population analyzed. In the present invention, the statistical methods and models can be selected to produce a desired clinical parameter for a clinical population with a particular IBD, UC, or CD prevalence. For example, statistical methods and models can be selected for an IBD, UC, or CD prevalence of up to about 1%, 2%, 3%, 4%, 5%, 6%, 7%, 8%, 9%, 10%, 15%, 20%, 25%, 30%, 35%, 40%, 45%, 50%, 55%, 60%, 65%, or 70%, which can be seen, e.g., in a clinician's office such as a gastroenterologist's office or a general practitioner's office.

As used herein, the term “overall agreement” or “overall accuracy” refers to the accuracy with which a method of the present invention diagnoses IBD, diagnoses UC, or differentiates between UC and CD. Overall accuracy is calculated as the sum of the true positives and true negatives divided by the total number of sample results and is affected by the prevalence of the disease in the population analyzed. For example, the statistical methods and models can be selected such that the overall accuracy in a patient population having a disease prevalence is at least about 40%, and can be, e.g., at least about 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%.

### IX. Kits

The present invention provides kits for determining the presence or absence of one or more of the SNPs described herein. In certain aspects, the kits of the invention comprise one or more probes. In particular embodiments, the kits comprise:

(i) a first labeled probe capable of binding to the wild-type variant allele of a target polynucleotide comprising a SNP location (or site); and

(ii) a second labeled probe capable of binding to a non-wild-type variant allele of the target polynucleotide comprising the SNP location (or site),

wherein the first and second probes are differentially labeled.

Differential labeling allows for separate detection of probes within a single reaction mixture. For the methods of the present invention, each allelic version of the probe is labeled with a different dye, thereby allowing for detection of both the wild-type and mutant probes. Examples of dye-labeled probes include, but are limited to, VIC™ or FAM dye-labeled TaqMan probes (available from Applied Biosystems, USA). Additional examples of dyes for labeling probes include, but are not limited to, Cy3; Cy3.5; Cy5; Cy5.5; 5-FAM; 6-FAM; 5(6)-FAM; 5-FAM, SE; 6-FAM, SE; 5(6)-FAM, SE; 5-TAMRA; 6-TAMRA; 5(6)-TAMRA; 5-TAMRA, SE; 6-TAMRA, SE; 5(6)-TAMRA, SE; dR110 5-FAM™ 6-FAM™ 6-FAM 5-FAM 6-FAM 6-FAM 6-FAM; Green Dyes (including, e.g., dR6G; JOE™; HEX™; VIC®; JOE; VIC; TET™; dR6G); Yellow Dyes (including, e.g., dTAMRA™; TAMRA™; NED™; NED; HEX); Red Dyes (including, e.g., dROX™; ROX™; ROX; PET®; TAMRA) and Orange Dyes (including, e.g., LIZ® and LIZ).

In some embodiments, the probe sequences for inclusion in the kit used to detect SNP rs228224 are: TACCAGAGTCCCAAGTTTCTGGGGGATTCCCAGGTTAGCCCAAGCCGTGCT (SEQ ID NO:39) and TACCAGAGTCCCAAGTTTCTGGGGGGTTCCCAGGTTAGCCCAAGCCGTGCT (SEQ ID NO:39), both derived from TACCAGAGTCCCAAGTTTCTGGGGG[A/G]TTCCCAGGTTAGCCCAAGCCGTGCT (SEQ ID NO:39), wherein the notation [A/G] represents the location of the rs2228224 SNP. In further embodiments, the first probe is VIC™ dye labeled and contains the A allele and the second probe is FAM™ labeled and contains the G allele. For detecting the presence or the absence of the rs2228224 SNP, a FAM/FAM (G/G) signal would indicate a homozygous wild-type genotype; a VIC/VIC (A/A) signal would indicate a homozygous mutant genotype; and a VIC/FAM signal would indicate a heterozygous mutant genotype.

In some embodiments, the probe sequences for inclusion in the kit used to detect SNP rs2032582 are: TATTTAGTTTGACTCACCTTCCCAGCACCTTCTAGTTCTTTCTTATCTTTC (SEQ ID NO:40) and TATTTAGTTTGACTCACCTTCCCAGAACCTTCTAGTTCTTTCTTATCTITC (SEQ ID NO:40); both derived from TATTTAGTTTGACTCACCTTCCCAG[C/A]ACCTTCTAGTTCTTTCTTATCTTTC (SEQ ID NO:40); wherein the notation [C/A] represents the location of the rs2032582 SNP. In further embodiments, the first probe is VIC™ dye labeled and contains the C allele and the second probe is FAM™ labeled and contains the A allele. For detecting the presence or the absence of the rs2032582 SNP the probe is reversed (G for C and T for A), as such a FAM/FAM (T/T) signal would indicate a homozygous wild-type genotype; a VIC/VIC (G/G) signal would indicate a homozygous mutant genotype; and a VIC/FAM signal would indicate a heterozygous mutant genotype.

In some embodiments, the probe sequences for inclusion in the kit used to detect SNP rs2032582 are: TATTTAGTTTGACTCACClTCCCAGCACCTTCTAGTTCTTTCTTATCTTTC (SEQ ID NO:41) and TATTTAGTTTGACTCACCTTCCCAGTACCTTCTAGTTCTTTCTTATCTTTC (SEQ ID NO:41); both derived from TATTTAGTTTGACTCACCTTCCCAG[C/T]ACCTTCTAGTTCTTTCTTATCTTTC (SEQ ID NO:41); wherein the notation [C/T] represents the location of the rs2032582 SNP. In further embodiments, the first probe is VIC™ dye labeled and contains the C allele and the second probe is FAM™ labeled and contains the T allele. For detecting the presence or the absence of the rs2032582 SNP the probe is reversed (G for C and T for A), as such a FAM/FAM (A/A) signal would indicate a homozygous wild-type genotype; a VIC/VIC (G/G) signal would indicate a homozygous mutant genotype; and a VIC/FAM signal would indicate a heterozygous mutant genotype.

In some embodiments, the probe sequences for inclusion in the kit used to detect SNP rs2241880 are: CCCAGTCCCCCAGGACAATGTGGATACTCATCCTGGTTCTGGTAAAGAAGT (SEQ ID NO:42) and CCCAGTCCCCCAGGACAATGTGGATGCTCATCCTGGTTCTGGTAAAGAAGT (SEQ ID NO:42), derived from CCCAGTCCCCCAGGACAATGTGGAT[A/G]CTCATCCTGGTTCTGGTAAAGAAGT (SEQ ID NO:42); wherein the notation [A/G] represents the location of the rs2241880 SNP. In further embodiments, the first probe is VIC™ dye labeled and contains the A allele and the second probe is FAM™ labeled and contains the G allele. For detecting the presence or the absence of the rs2241880 SNP, a FAM/FAM (G/G) signal would indicate a homozygous wild-type genotype; a VIC/VIC (A/A) signal would indicate a homozygous mutant genotype; and a VIC/FAM signal would indicate a heterozygous mutant genotype.

In some embodiments, the kits contain one or more sets of probes. In other embodiments, the kits may contain buffers or other reagents necessary for the SNP detection reactions. The types of buffers and other reagents are well-known in the art and their use can be readily determined by one skilled in the art.

### X. Examples

The following examples are offered to illustrate, but not to limit the claimed invention.

### Example 1

### DNA Isolation Methods

The samples used for DNA isolation were obtained from blood or body fluids using standard procedures known in the art. For DNA isolation from the samples, the QIAGEN Protocol for DNA Purification from Blood or Body Fluids (Spin Protocol) in the 100 μl reaction size was employed using the supplied protocols (QIAamp DNA Blood Mini Kit, Catalog #51106 obtained from QIAGEN, USA).

DNA Isolation Procedure:
1) Pipet 20 μl Protease into the bottom of a 1.5 ml microcentrifuge tube.
2) Add 100 μl sample to the microcentrifuge tube.
3) Add 100 μl 1×PBS to the microcentrifuge tube.
4) Add 200 μl Buffer AL to the sample.
5) Mix by pulse-vortexing for 15 sec.
6) Incubate at 56° C. for 10 min.
7) Briefly centrifuge the 1.5 ml microcentrifuge tube to remove drops from the inside of the lid.
8) Add 200 μl ethanol (96-100%) to the sample.
9) Mix by pulse-vortexing for 15 sec.
10) Briefly centrifuge the 1.5 ml microcentrifuge tube to remove drops from the inside of the lid.
11) Carefully apply the mixture to a QIAamp Mini spin column (in a 2 ml collection tube) without wetting the rim. Close cap.
12) Centrifuge at 6000×g (8000 rpm) for 1 min.
13) Place the spin column in a clean 2 ml collection tube, discard tube containing the filtrate.
14) Carefully open the spin column and add 500 μl Buffer AW1 without wetting the rim. Close cap.
15) Centrifuge at 6000×g (8000 rpm) for 1 min.
16) Place the spin column in a clean 2 ml collection tube, discard tube containing the filtrate.
17) Carefully open the spin column and add 500 μl Buffer AW2 without wetting the rim. Close cap.
18) Centrifuge at full speed 20000×g (14,000 rpm) for 3 min.
19) Place the spin column in a clean 1.5 ml microcentrifuge tube, and discard tube containing the filtrate.
20) Open spin column and add 200 μl Buffer AE.
21) Incubate at room temperature for 5 min.
22) Centrifuge at 6000×g (8000 rpm) for 1 min.

### Example 2

### SNP Assay Methods

For SNP analysis, the ABI 384 Fast Real-Time Plate Prep Kit was used (Applied Biosystems, USA). Briefly, the assay materials consisted of TaqMan GTXpress Master Mix and ABI Genotyping assay appropriate for each SNP (for rs2228224, the Assay ID used was C_{—}3125146_{—}10; for rs2228226, the Assay ID used was C_{—}11293074_{—}10; for rs2032582, the Assay ID used was C_{—}11711720D_{—}30 or C_{—}11711720D_{—}40; and for rs2241880, the Assay ID used was C_{—}9095577_{—}20). Additional assay materials included: AXYGEN Scientific Reservoir 8 Row (Part Number RES-MW8-LP-SI; Axygen Biosciences, California, USA); ABI MicroAmp Optical 384-Well Reaction Plate with Barcode (Part Number 4309849, from Applied Biosystems, USA); MicroAmp Optical Adhesive Film (Part Number 4311971, from Applied Biosystems, USA). The system used for PCR reactions was the 7900HT Fast Real-Time PCR System E2216 (Applied Biosystems, USA). Products were used according to accompanying manufacturers instructions.

SNP Detection Procedure:

1) Thaw Genotyping assay mix on ice. Keep genotyping mix protected from light.

2) Keep GTXpress Master Mix on ice. Keep master mix protected from light.

3) Add sample/control DNA to assigned plate well:
a. When using 40× genotyping mix: add 2.375 μl DNA/well
OR
b. When using 20× genotyping mix: add 2.25 μl DNA/well

4) Preparing Reaction (Rxn) Mix:
a. Gently invert GTXpress Master Mix to mix contents.
b. Gently vortex the Genotyping assay to mix contents and spin contents down by briefly centrifuging.
c. In sterile cryovial, pipette in first the GTXpress Master Mix, then the genotyping assay:
   i. GTXpress Master Mix amount: add 2.5 μl/well
   ii. Genotyping assay amount:
      1. When using 40× genotyping mix: add 0.125 μl genotyping mix/well
      OR
      2. When using 20× genotyping mix: add 0.25 μl genotyping mix/well

5) Gently vortex cryovial to mix contents.

6) Pour contents of cryovial into sterile reservoir then pipette:
a. When using 40× genotyping mix: pipette 2.625 μl r×n mix/well
   OR
   When using 20× genotyping mix: pipette 2.75 μl r×n mix/well

7) Seal plate.

8) Vortex plate, then tap plate to remove any existing air bubbles in the well.

9) Set Sample Volume=5 μl and Start RT PCR:
a. Stage 1: 50.0° C. for 2:00 min
b. Stage 2: 95.0° C. for 10:00 min
c. Stage 3: Repeats: 40
   i. 95.0° C. for 0:15 min
   ii. 60.0° C. for 1:00 min

### Example 3

### Genetic Variants Combined with Serological Markers Improve Ulcerative Colitis Identification

Crohn's Disease (CD) and Ulcerative Colitis (UC) are two common forms of inflammatory bowel disease (IBD). Serological markers can be used to help distinguish these diseases, although their accuracy is generally greater for CD. This is due to the fact that most of the serological markers are found in CD patients whereas there is only one predominant UC marker, namely, anti-neutrophil cytoplasmic antibodies (ANCA). UC-associated ANCA yields a perinuclear staining pattern (pANCA) on alcohol fixed neutrophils. However despite its high specificity, only 48% of the UC cases are pANCA positive. The search for new IBD markers using GWAS analysis confirmed that genetic mutations are playing an important role in the disease. Indeed, numerous genetic markers have been identified and associated with CD, UC or both.

Purpose of the Study:

The aim of this study was to identify genetic markers that can contribute, in combination with ANCA/pANCA, to better identify patients with UC.

Methods:

DNA from well-characterized UC patients (n=81) and healthy control (HC, n=153) were genotyped for variants in three genes: GLI1 (rs2228224), MDR1 (rs2032582), and ATG16L1 (rs2241880). Differences in risk allele frequencies between UC and HC were analyzed using Fisher's exact test, and odds ratio (OR) were calculated with 95% confidences intervals (CIs). Patient and control serum were tested for ANCA by ELISA and pANCA by immunofluorescence followed by DNAse treatment on fixed neutrophils. Predictive models were generated using random forests and validated using leave-one-out cross validation.

Results:

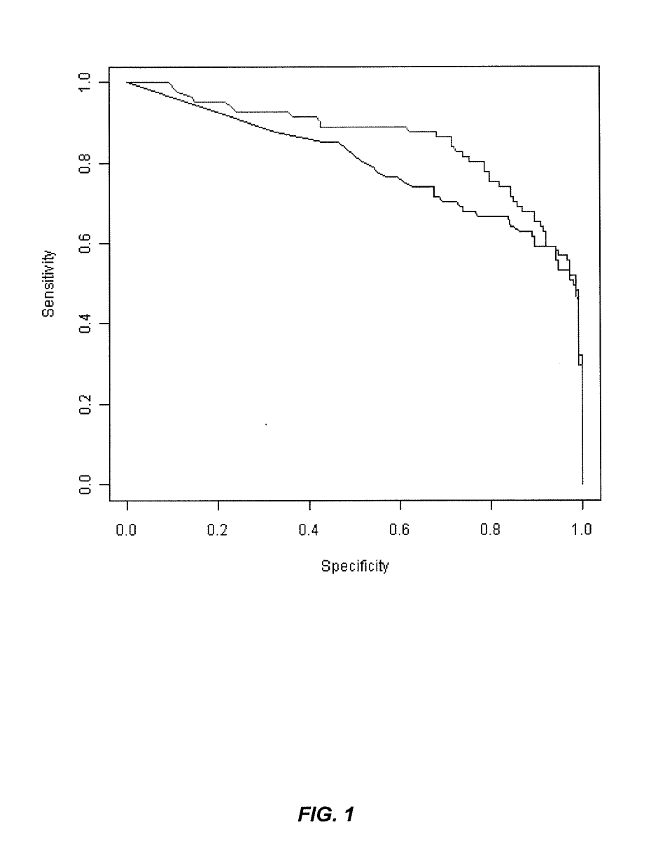

Significant differences in risk allele frequency were found for the GLI1 (G933D) mutation in UC compared to HC (p<0.001, OR=2.64, 95% CI=1.73-4.07). For the triallelic MDR1 variants, the most common MDR1 mutation (A893S) was found to be significantly associated with UC (p=0.010, OR=1.67, 95% CI=1.11-2.51). ATG16L1 was significantly associated with UC as well (p=0.006, OR=1.73, 95% CI=1.16-2.59) (Table 3). Receiver operator characteristic (ROC) analysis was used to compare the diagnostic accuracy of ANCA/pANCA alone to the three gene variants combined with ANCA/pANCA (FIG. 1). The addition of the three gene variants increased the area under the ROC curve from 0.793 (CI=0.726-0.861) to 0.856 (CI=0.799-0.912) which consequently improved the ANCA/pANCA sensitivity from 67% to 78% at a fixed specificity of 80% (Table 4).

Conclusions:

We have characterized a new genetic variation, GLI1 (G933D), associated with UC and confirmed the association of MDR1 (A893S) and ATG16L1 (T300A) variants with UC. These genetic variants, in combination with ANCA/pANCA, provided greater diagnostic accuracy for UC than ANCA/pANCA alone.

TABLE 3
SNP Association with UC

| | | RAF (HC vs UC) | |
|---|---|---|---|
| Genes | SNPs | P-value | OR (CI) |
| GLI1 (G933D) | rs2228224 | <0.001 | 2.64 (1.73-4.07) |
| MDR1 (A893S) | rs2032582 | 0.010 | 1.67 (1.11-2.51) |
| ATG16L1(T300A) | rs2241880 | 0.006 | 1.73 (1.16-2.59) |

TABLE 4
Addition of Gene Variants to ANCA/pANCA

| | HC | UC | |
|---|---|---|---|
| | (specificity) | (sensitivity) | AUC (CI) |
| Serology (ANCA + | 80% | 67% | 0.793 (0.726-0.861) |
| pANCA) | | | |
| Serology + 3 gene | 80% | 78% | 0.856 (0.799-0.912) |
| variants | | | |

### Example 4

### Combining Genetic Variants with Serological Markers Improves the Accuracy in the Diagnosis of Ulcerative Colitis

Crohn's Disease (CD) and Ulcerative Colitis (UC) are two common forms of inflammatory bowel disease (IBD). Serological markers can be used to help distinguish these diseases, although their accuracy is generally greater for CD. This is due to the fact that most of the diverse serologic markers are associated with CD whereas there is only one predominant UC marker, namely, anti-neutrophil cytoplasmic antibodies (ANCA). UC-associated ANCA yields a perinuclear staining pattern (pANCA) on alcohol fixed neutrophils. However, despite its high specificity, only 48% of the UC cases are pANCA positive. The search for new IBD markers using GWAS analysis confirmed that genetic mutations are playing an important role in the disease etiology. Numerous genetic markers have been identified and associated with CD, UC or both.

Purpose of the Study:

The aim of this exploratory study was to identify genetic markers that can contribute, in combination with ANCA/pANCA, to better identify patients with UC.

Methods:

DNA from well-characterized UC patients (n=81) and healthy control (HC, n=153) were genotyped for variants in two genes: GLI1 (rs2228224) and MDR1 (rs2032582). Differences in risk allele frequencies between UC and HC were analyzed using Fisher's exact test, and odds ratio (OR) were calculated with 95% confidences intervals (CIs). Patient and control serum were tested for ANCA by ELISA and pANCA by immunofluorescence followed by DNAse treatment on fixed neutrophils. Predictive models were generated using random forests and validated using leave-one-out cross validation.

Results:

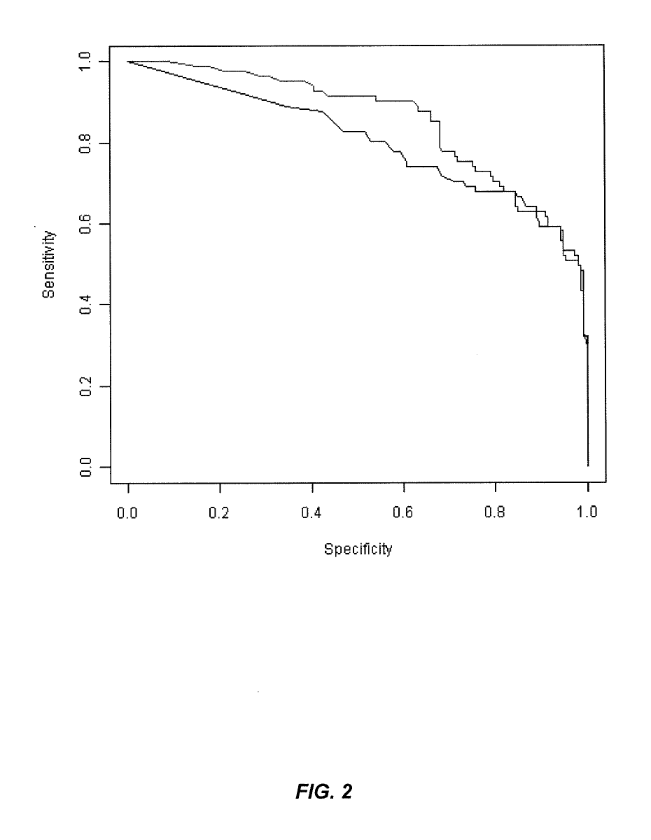

Significant differences in risk allele frequency were found for the GLI1 (G933D) mutation in UC compared to HC (p<0.001, OR=2.64, 95% CI=1.73-4.07). For the triallelic MDR1 variants, the most common MDR1 mutation (A893S) was significantly associated with UC (p=0.010, OR=1.67, 95% CI=1.11-2.51) (Table 5). Receiver operator characteristic (ROC) analysis was used to compare the diagnostic accuracy of ANCA/pANCA alone to the two gene variants combined with ANCA/pANCA (FIG. 2). The addition of the two gene variants increased the area under the ROC curve from 0.793 (CI=0.726-0.861) to 0.853 (CI=0.801-0.905) (Table 6).

Conclusions:

We have characterized a new genetic variation, GLI1 (G933D), associated with UC and confirmed the association of MDR1 (A893S) variants with UC. In this population subset, these genetic variants, in combination with ANCA/pANCA, provided greater diagnostic accuracy for UC than ANCA/pANCA alone.

TABLE 5
SNP Association with UC

| | | RAF (HC vs UC) | |
|---|---|---|---|
| Genes | SNPs | P-value | OR (CI) |
| GLI1 (G933D) | rs2228224 | <0.001 | 2.64 (1.73-4.07) |
| MDR1 (A893S) | rs2032582 | 0.010 | 1.67 (1.11-2.51) |

TABLE 6
Addition of Gene Variants to ANCA/pANCA

| | HC | UC | |
|---|---|---|---|
| | (specificity) | (sensitivity) | AUC (CI) |
| Serology (ANCA + | 80% | 68% | 0.793 (0.726-0.861) |
| pANCA) | | | |
| Serology + 2 gene | 80% | 72% | 0.853 (0.801-0.905) |
| variants | | | |

### Example 5

### Combining Genetic Variants with Serological Markers Improves the Accuracy in the Diagnosis of Ulcerative Colitis

### Introduction

Inflammatory Bowel Disease (IBD) is composed of several disorders in which the lining of the bowel is continuously or repeatedly inflamed. The causes of IBD are unclear, but are believed to be polygenic in nature and involve erroneous recognition by the immune system of tissues lining the bowel and accumulation of immune system cells in the lining of the bowel resulting in inflammation. Two common forms of IBD are Ulcerative Colitis (UC) and Crohn's Disease (CD). Distinguishing between UC and CD can be achieved by the examination of serological markers. Most serological markers are associated with CD (e.g., ASCA IgA and IgG, anti-OmpC, anti-CBir1, anti-I2, etc.). Only anti-neutrophil cytoplasmic antibodies (ANCA) are predominantly found with UC. In 48% of UC cases, alcohol-fixed neutrophils produce a perinuclear staining pattern (pANCA), rendering pANCA specific but not sensitive for UC. Genome-wide association studies (GWAS) have identified numerous susceptibility loci for IBD, including a linkage region on chromosome 7q containing the multidrug resistance gene (ABCB1/MDR1) (Brant et al., Am J Hum Genet., 2003; 73(6) 1282-1292.) and the IBD2 linkage region 12q13 containing glioma-associated oncogene homolog 1 (Gli1) (Lees et al., PLoS Med., 2008; 5(12) E239).

### Purpose of the Study

To identify new genetic markers that contribute, in combination with ANCA/pANCA, to diagnostic tests that more successfully identify patients with UC.

### Materials and Methods

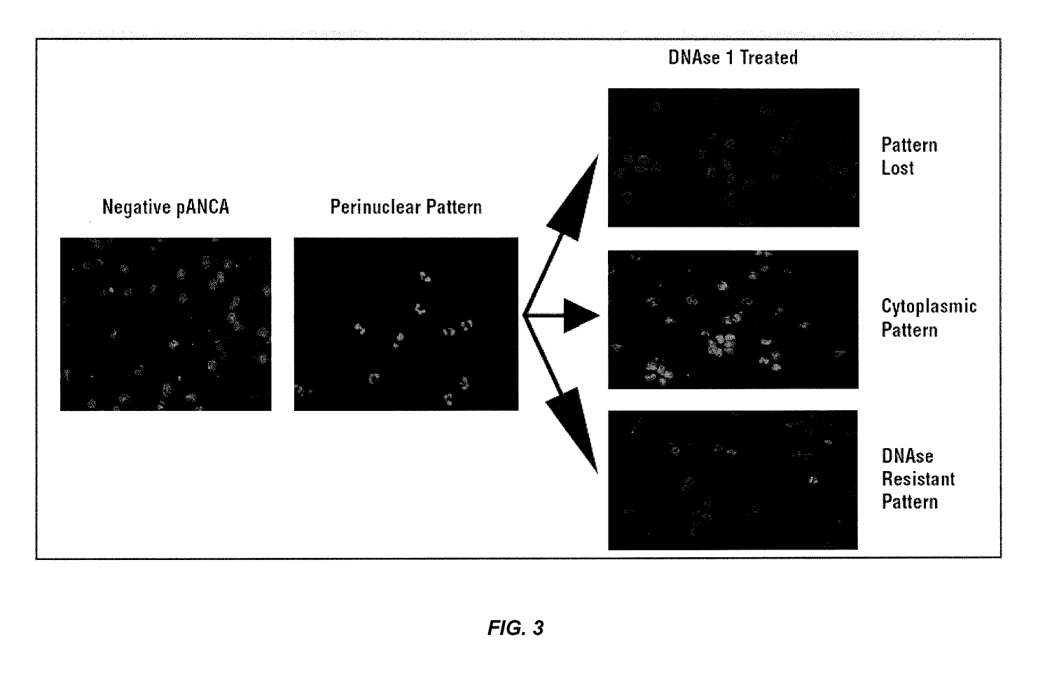

DNA from well-characterized UC patients (n=81), and healthy controls (HC, n=153) was genotyped for three variants in two genes: GLI1 (rs2228224 and rs2228226) and MDR1 (rs2032582) (Table 7). Differences in risk allele frequencies (RAF) between UC and HC were analyzed using Fisher's exact test, and odds ratios (OR) were calculated with 95% confidences intervals (CIs). Patient and control serum was tested for ANCA by ELISA and pANCA by immunofluorescence followed by DNAse treatment on fixed neutrophils (FIG. 3). Predictive models were generated using random forests and validated using leave-one-out cross validation.

TABLE 7
Patient Characteristics

| | Ulcerative Colitis | | Healthy Control |
|---|---|---|---|
| Characteristic | (n = 81) | | (n = 153) |
| Gender | 39% Male | | 43% Male |
| Average Diagnostic Age (yr) | 30 | | N/A |
| Disease Extent/Location | n = | % | |
| Cecum | 37 | 46 | N/A |
| Ascending Colon | 47 | 59 | N/A |
| Transverse Colon | 53 | 66 | N/A |
| Descending Colon | 66 | 83 | N/A |
| Sigmoid | 74 | 93 | N/A |
| Rectum | 80 | 100 | N/A |

### Results

The distribution of pANCA/ANCA markers was higher in the UC population compared to healthy controls (Table 8):
1% of HC samples were pANCA positive.
48% of UC samples were pANCA positive.
10% of the HC samples had high serum ANCA values.
60% for the UC samples had high serum ANCA values.

Significant differences in RAF were found for the GLI1 and MDR1SNPs in UC vs. HC (Table 9):
GLI1 (G933D) p<0.001, OR: 2.64, (95% CI: 1.73-4.07).
GLI1 (Q 1100E) p=0.02, OR: 1.66, (95% CI: 1.07-2.62).
MDR1 (A893S) p=0.01, OR: 1.67, (95% CI: 1.11-2.51).

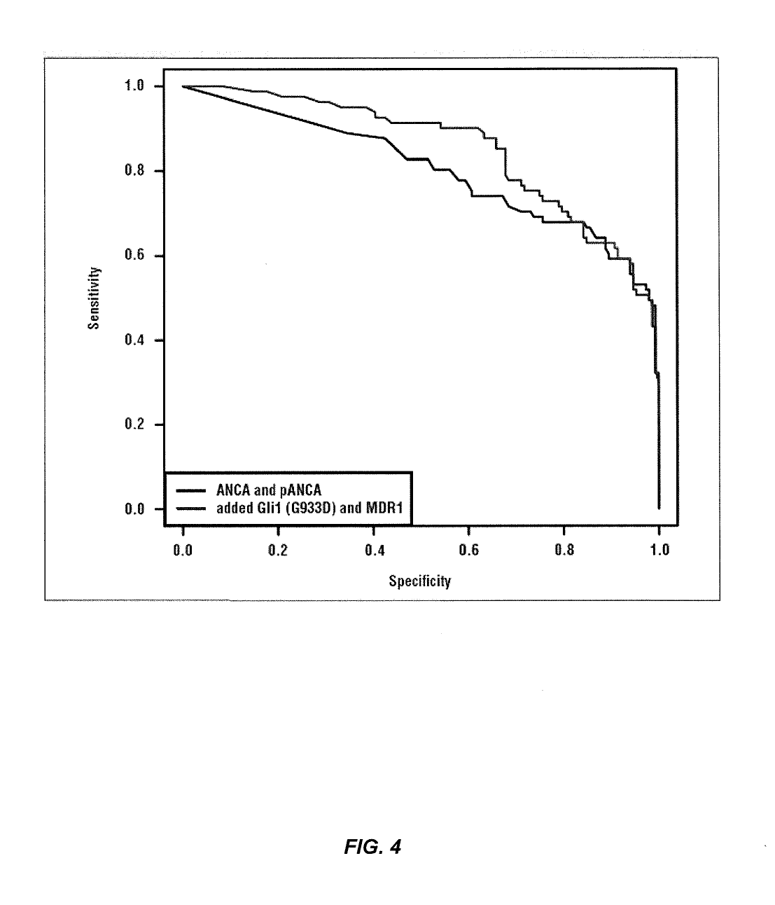

Addition of two gene variants to ANCA/pANCA increased the area under the curve from 0.802 (95% CI: 0.737-0.868) to 0.853 (95% CI: 0.801-0.905) (FIG. 4).

TABLE 8
Distribution of ANCA/pANCA Markers

| | | | | | ANCA | | ANCA | |
|---|---|---|---|---|---|---|---|---|
| | pANCA− | | pANCA+ | | Low | | High | |
| | n = | % | n = | % | n = | % | n = | % |
| Healthy Control | 151 | 99 | 2 | 1 | 137 | 90 | 16 | 10 |
| (n = 153) | | | | | | | | |
| Ulcerative Colitis | 42 | 52 | 39 | 48 | 32 | 40 | 49 | 60 |
| (n = 81) | | | | | | | | |

TABLE 9
Risk Allele Frequency for GLI1 and MDR1 SNP

| | Healthy | | Ulcerative | | | |
|---|---|---|---|---|---|---|
| | Control | | Colitis | | | |
| | n = | % | n = | % | P-Value | OR (CI) |
| Gli1 (Q1100E) | 193 | 63.9 | 121 | 74.7 | 0.02 | 1.66 (1.07-2.62) |
| rs2228226 | | | | | | |
| Gli1 (G933D) | 147 | 48 | 115 | 71 | <0.0001 | 2.64 (1.73-4.07) |
| rs2228224 | | | | | | |
| MDR1 | 111 | 36.2 | 79 | 48.8 | 0.01 | 1.67 (1.11-2.51) |
| (S893A/T) | | | | | | |
| rs2032582 | | | | | | |

The ABCB1/MDR1 gene is located on chromosome 7q21.12. The triallelic genetic variation 2677G>T/A (rs2032582) in exon 21 leads to intracellular non-synonymous amino acid change in position 893 (A893S/T). See, Wang et al., AAPS J., 2006; 8(3) E515-E520. The GLI1 gene is located on chromosome 12q13.2-q13.3. The genetic variation 3376G>C (rs2228226) in exon 12 leads to non-synonymous amino acid change in position 1100 (Q1100E). The genetic variation 2876G>A (rs2228224) leads to non-synonymous amino acid change in position 933 (G933D). See, Lees et al., PLoS Med., 2008; 5(12) E239.

Anti-neutrophil cytoplasmic antibodies (ANCAs) are directed against intracellular components of neutrophils (FIG. 3). Confocal and electron microscopy demonstrated that UC associated pANCA was localized primarily over chromatin, concentrated toward the periphery of the nuclei. In UC patients, after treatment with DNAse I, the pANCA staining pattern was lost. In approximately 70% of UC cases, there was complete loss of antigen recognition, while in 30% of cases there was conversion to cytoplasmic staining. Three percent of UC patients have a resistant pattern (Nakamura et al., Clin Chim Acta., 2003; 335(1-2) 9-20).

As expected, the distribution of pANCA/ANCA markers was higher in the UC population compared to HC. Indeed, only 1% of HC samples compared to 48% of UC samples were pANCA positive. Similarly, only 10% of the HC samples compared to 60% of UC samples had high serum ANCA values (Table 8).

A significant difference in RAF was found for the GLI1 (G933D) mutation in UC compared to HC (p<0.001, OR: 2.64, 95% CI: 1.73-4.07). A significant RAF difference was also found for GLI1 (Q1100E) (p=0.02, OR: 1.66, 95% CI: 1.07-2.62). For the triallelic MDR1 variants, the most common MDR1 mutation (A893S) was significantly associated with UC (p=0.010, OR: 1.67, 95% CI: 1.11-2.51) (Table 9).

Receiver Operator Characteristic analysis was used to compare the diagnostic accuracy of ANCA/pANCA alone to the two gene variants, GLI1 (G933D) and MDR1 (A893S) combined with ANCA/pANCA. The addition of the two gene variants increased the area under the curve from 0.802 (95% CI: 0.737-0.868) to 0.853 (95% CI: 0.801-0.905) (FIG. 4).

### Conclusions

This study has characterized a new UC-associated genetic variation: GLI1 (G933D), and has confirmed the association of MDR1 (A893S) variants with UC. In this population subset, GLI1 and MDR1 variants in combination with ANCA/pANCA provided greater diagnostic accuracy for UC than ANCA/pANCA alone.

### Example 6

### Risk Allele Factor (RAF) Analysis for GLI1 (G933D) rs2228224 and MDR1 (A893S/T) rs2032582

This example provides an analysis of the association between the GLI1 (G933D) rs2228224 and MDR1 (A893S/T) rs2032582 SNPs and ulcerative colitis (UC) in samples from Crohn's Disease (CD), UC, and Healthy Control (HC) patients.

The detection of the rs2228224 SNP was performed as described herein. For assay result interpretation for the rs2228224 SNP analysis, a FAM/FAM (G/G) signal was indicated as homozygous wild-type; a VIC/VIC (A/A) signal was indicated as homozygous mutant; and a VIC/FAM signal was indicated as heterozygous mutant.

The detection of the rs2032582 SNP was performed as described herein. For assay result interpretation for the rs2032582 SNP analysis, a FAM/FAM (T/T) signal was indicated as homozygous mutant; a VIC/VIC (G/G) signal was indicated as a homozygous wild-type genotype and a VIC/FAM signal was indicated as a heterozygous mutant genotype.

Table 10 below shows the Risk Allele Frequency for GLI1 (G933D) rs2228224 and MDR1 (A893S) rs2032582. In particular, Table 10 contains data for comparison of Healthy Control (HC) to Ulcerative Colitis (UC), HC to Crohn's Disease (CD), and CD to UC.

TABLE 10

| | HC | | UC | | | |
|---|---|---|---|---|---|---|
| | n | % | n | % | p-Value | OR (CI) |
| Gli1 | 209 | 49% | 206 | 68% | <0.00001 | 2.21 (1.48-3.30) |
| (G933D) | | | | | | |
| rs2228224 | | | | | | |
| MDR1 | 114 | 26.5% | 143 | 48% | <0.00001 | 1.94 (1.40-2.68) |
| (A893S) | | | | | | |
| rs2032582 | | | | | | |
| | HC | | CD | | | |
| | n | % | n | % | p-Value | OR (CI) |
| Gli1 | 209 | 49% | 325 | 59% | <0.001 | 1.56 (1.21-2.01) |
| (G933D) | | | | | | |
| rs2228224 | | | | | | |
| MDR1 | 114 | 26.5% | 199 | 38% | 0.07 | 1.29 (0.97-1.72) |
| (A893S) | | | | | | |
| rs2032582 | | | | | | |
| | CD | | UC | | | |
| | n | % | n | % | p-Value | OR (CI) |
| Gli1 | 325 | 59% | 206 | 68% | 0.01 | 1.42 (1.06-1.88) |
| (G933D) | | | | | | |
| rs2228224 | | | | | | |
| MDR1 | 199 | 38% | 143 | 48% | 0.01 | 1.5 (1.12-2.01) |
| (A893S) | | | | | | |
| rs2032582 | | | | | | |

Table 10 shows that the GLI1 (G933D) rs2228224 and MDR1 (A893S) rs2032582 variant alleles were each independently and significantly associated with UC compared to HC or CD. In addition, Table 10 shows that the GLI1 (G933D) rs2228224 variant allele was significantly associated with CD compared to HC. As such, determining the presence or absence of the GLI1 (rs2228224) and/or MDR1 (rs2032582) variant alleles in accordance with the present invention is particularly useful for diagnosing of UC, e.g., by identifying patients as having UC versus healthy control patients and/or patients with CD.

Examples 7-9 describe an analysis of additional samples to determine the presence or absence of the GLI1 (G933D) rs2228224, MDR1 (A893S/T) rs2032582, and ATG16L1 (T300A) rs2241880 SNPs.

### Example 7

### Detection of GLI1 (G933D) rs2228224

The GLI1 gene is located on Chromosome 12. The rs2228224 SNP is a mis-sense mutation consisting of a transition from G to A with a codon change of GGT to GAT. The rs2228224 SNP is located at position 2876 on the transcript NM_{—}005269.2 (SEQ ID NO:25). The transition leads to an amino acid change G993D (glycine 933 to aspartic acid) on the protein ID NP_{—}005260.1 (SEQ ID NO:26).

For detection of the rs2228224 SNP, the ABI TaqMAN assay was used (Applied Biosystems). The ABI Assay ID number was C_{—}3125146_{—}10 (available from Applied Biosystems, USA). The following context sequence was used for the TaqMan assay [VIC/FAM]: TACCAGAGTCCCAAGTTTCTGGGGG[A/G]TTCCCAGGTTAGCCCAAGCCGTGCT (SEQ ID NO:39) The notation [A/G] represents the location of the rs2228224 SNP. The VIC version of the probe contains the A allele and the FAM probe contains the G allele.

For assay result interpretation for the rs2228224 SNP analysis, a FAM/FAM (G/G) signal was indicated as homozygous wild-type; a VIC/VIC (A/A) signal was indicated as homozygous mutant; and a VIC/FAM signal was indicated as heterozygous mutant. These results are shown in Table 11.

TABLE 11
GLI1 G933D (C 3125146 10; rs2228224)

| Diagnosis | Count | VIC (A) | VIC % | BOTH | BOTH % | FAM (G) | FAM % |
|---|---|---|---|---|---|---|---|
| IBD CROHN'S DISEASE | 547 | 197 | 36.0% | 257 | 47.0% | 93 | 17.0% |
| IBD ULCERATIVE COLITIS | 304 | 141 | 46.4% | 130 | 42.8% | 33 | 10.9% |
| HC/HEALTHY CONTROL/NORMAL | 428 | 117 | 27.3% | 185 | 43.2% | 126 | 29.4% |
| IBS GI Control | 149 | 46 | 30.9% | 71 | 47.7% | 32 | 21.5% |

The results in the following Tables 12-17 represent the Risk Allele Factor (RAF) analyses for the rs2228224 SNP. The risk allele is A. The p values were calculated using the frequency of both alleles after the heterozygous mutant values were split and equally redistributed in both homozygous wild-type and homozygous mutant genotypes. The different populations (Crohn's Disease (CD), Ulcerative Colitis (UC), Healthy Control (HC), and IBS GI Control (IBS)) were then compared between each other as indicated. Table 12 contains data for comparison of HC to UC. Table 13 contains data for comparison of HC to CD. Table 14 contains data for comparison of CD to UC. Table 15 contains data for comparison of IBS to UC. Table 16 contains data for comparison of IBS to CD. Table 17 contains data for comparison of IBS to HC.

TABLE 12
RAF Analysis for HC vs. UC

| | | |
|---|---|---|
| P value | 9.26E−05 | (95% Confidence) |

| | | | |
|---|---|---|---|
| Odds Ratio | 2.211735 | 1.480269 | 3.30465 |

TABLE 13
RAF Analysis for HC vs. CD

| | | |
|---|---|---|
| P value | 0.000609 | (95% Confidence) |

| | | | |
|---|---|---|---|
| Odds Ratio | 1.561224 | 1.209452 | 2.015312 |

TABLE 14
RAF Analysis for CD vs. UC

| | | |
|---|---|---|
| P value | 0.016385 | (95% Confidence) |

| | | | |
|---|---|---|---|
| Odds Ratio | 1.416667 | 1.065424 | 1.883705 |

TABLE 15
RAF Analysis for IBS vs. UC

| | | |
|---|---|---|
| P value | 0.006857 | (95% Confidence) |

| | | | |
|---|---|---|---|
| Odds Ratio | 1.738636 | 1.162187 | 2.601006 |

TABLE 16
RAF Analysis for IBS vs. CD

| | | |
|---|---|---|
| P value | 0.271411 | (95% Confidence) |

| | | | |
|---|---|---|---|
| Odds Ratio | 1.227273 | 0.851724 | 1.768411 |

TABLE 17
RAF Analysis for IBS vs. HC

| | | |
|---|---|---|
| P value | 0.207079 | (95% Confidence) |

| | | | |
|---|---|---|---|
| Odds Ratio | 0.786096 | 0.540662 | 1.142946 |

Tables 12, 14, and 15 show that the GLI1 (G933D) rs2228224 variant allele was significantly associated with UC compared to HC or CD or IBS. Table 13 shows that the GLI1 (G933D) rs2228224 variant allele was significantly associated with CD compared to HC. As such, determining the presence or absence of the GLI1 (rs2228224) variant allele in accordance with the present invention is particularly useful for diagnosing UC, e.g., by identifying patients as having UC versus healthy control patients, IBS GI control patients, and/or patients with CD.

For assay result interpretation for the rs2228226 SNP analysis, a FAM/FAM (G/G) signal was indicated as homozygous wild-type; a VIC/VIC (A/A) signal was indicated as homozygous mutant; and a VIC/FAM signal was indicated as heterozygous mutant. These results are shown in Table 18.

TABLE 18
GLI1 Q1100E (C 11293074 10; rs2228226)

| Diagnosis | Count | VIC | VIC % | BOTH | BOTH % | FAM | FAM % |
|---|---|---|---|---|---|---|---|
| IBD CROHN'S DISEASE | 235 | 114 | 48.5% | 94 | 40.0% | 27 | 11.5% |
| IBD ULCERATIVE COLITIS | 254 | 134 | 52.8% | 99 | 39.0% | 21 | 8.3% |
| HC/HEALTHY CONTROL/NORMAL | 409 | 174 | 42.5% | 185 | 45.2% | 50 | 12.2% |

The results in the following Tables 19-21 represent the Risk Allele Factor (RAF) analyses for the rs2228226 SNP. The risk allele is C. The p values were calculated using the frequency of both alleles after the heterozygous mutant values were split and equally redistributed in both homozygous wild-type and homozygous mutant genotypes. The different populations (Crohn's Disease (CD), Ulcerative Colitis (UC), and Healthy Control (HC)) were then compared between each other as indicated. Table 19 contains data for comparison of HC to UC. Table 20 contains data for comparison of HC to CD. Table 21 contains data for comparison of CD to UC.

TABLE 19
RAF Analysis for HC vs. UC

| | | |
|---|---|---|
| P value | 0.060996 | (95% Confidence) |

| | | | |
|---|---|---|---|
| Odds Ratio | 1.384615 | 0.984504 | 1.947336 |

TABLE 20
RAF Analysis for HC vs. CD

| | | |
|---|---|---|
| P value | 0.342064 | (95% Confidence) |

| | | | |
|---|---|---|---|
| Odds Ratio | 1.181141 | 0.837682 | 1.665423 |

TABLE 21
RAF Analysis for CD vs. UC

| | | |
|---|---|---|
| P value | 0.423769 | (95% Confidence) |

| | | | |
|---|---|---|---|
| Odds Ratio | 1.172269 | 0.794003 | 1.730741 |

### Example 8

### Detection of MDR1 (A893S/T) rs2032582

The gene is located on Chromosome 7. There are two mis-sense mutations, either a transversion from a G to a T with a codon change of GCT to TCT, corresponding to a change from alanine to serine, or a transversion from a G to an A with a codon change from GCT to ACT, corresponding to a change from alanine to threonine. The SNP location is 3095 on the transcript NM_{—}000927.3 (SEQ ID NO:27). It leads to a AA change S893T/A on the protein ID NP_{—}000918.2 (SEQ ID NO:28).

For detection of the rs2032582 SNP, the ABI TaqMAN assay was used (Applied Biosystems, USA). The ABI assay ID number was C_{—}11711720C_{—}30 (A893S) which is the common mutation (assay available from Applied Biosystems, USA). As there are three alleles, a triallelic assay was employed.

The following probe sequence was used for the TaqMAN assay with ABI assay ID C_{—}11711720C_{—}30 (A893S): TATTTAGTTTGACTCACCTTCCCAG[C/A]ACCTTCTAGTTCTTTCTTATCTTTC (SEQ ID NO:40). The notation C/A represents the location of the rs2032582 SNP and the VIC labeled version of the probe contains the C allele and the FAM labeled version of the probe contains the A allele.

Some TaqMan probes were designed using the negative DNA strand and the rs2032582 probe of SEQ ID NO:40 was made to the negative strand; in other words the SNP is G to T on the positive strand and the probe made to the negative strand contains a C or an A. As such, G is substituted for C and T is substituted for A. For assay result interpretation for the rs2032582 SNP analysis, a FAM/FAM (T/T) signal was indicated as homozygous mutant; a VIC/VIC (G/G) signal was indicated as a homozygous wild-type genotype and a VIC/FAM signal was indicated as a heterozygous mutant genotype.

The following probe sequence was used for the TaqMAN assay with ABI assay ID C_{—}11711720D_{—}40 (A893T): TATTTAGTTTGACTCACCTTCCCAG[C/T]ACCTTCTAGTTCTTTCTTATCTTTC (SEQ ID NO:41). The notation C/T represents the location of the rs2032582 SNP and the VIC labeled version of the probe contains the C allele and the FAM labeled version of the probe contains the T allele.

Some TaqMan probes were designed using the negative DNA strand and the rs2032582 probe of SEQ ID NO:41 was made to the negative strand; in other words the SNP is G to A on the positive strand and the probe made to the negative strand contains a C or a T. As such, G is substituted for C and T is substituted for A. For assay result interpretation for the rs2032582 SNP analysis, a FAM/FAM (A/A) signal was called as homozygous mutant; a VIC/VIC (G/G) signal was called as homozygous wild-type; and a VIC/FAM signal was called as heterozygous mutant. These results are indicated in Table 22.

TABLE 22
MDR1 S893T/A (rs2032582)

| Diagnosis | Count | AA | AA % | GA | GA % | GG | GG % | GT | GT % | TA | TA % | TT | TT % |
|---|---|---|---|---|---|---|---|---|---|---|---|---|---|
| HC/NORMAL | 429 | 1 | 0.2% | 19 | 4.4% | 155 | 36.1% | 138 | 32.2% | 11 | 2.6% | 45 | 10.5% |
| IBD CROHN'S DISEASE | 525 | 4 | 0.8% | 21 | 4.0% | 184 | 35.0% | 228 | 43.4% | 3 | 0.6% | 85 | 16.2% |
| IBD ULCERATIVE COLITIS | 297 | 0 | 0.0% | 8 | 2.7% | 75 | 25.3% | 135 | 45.5% | 3 | 1.0% | 76 | 25.6% |
| IBS GI Control | 149 | 3 | 2.0% | 1 | 0.7% | 52 | 34.9% | 62 | 41.6% | 3 | 2.0% | 28 | 18.8% |

The results in the following Tables 23-28 represent the Risk Allele Factor (RAF) for the most common mutation A893S. The risk allele is T. The p values were calculated using the frequency of both alleles after the heterozygous mutant values were split and equally redistributed in both homozygous wild-type and homozygous mutant genotypes. The different populations (Crohn's Disease (CD), Ulcerative Colitis (UC), Healthy Control (HC), and IBS GI Control (IBS)) were then compared between each other. Table 23 contains data for comparison of HC to UC. Table 24 contains data for comparison of HC to CD. Table 25 contains data for comparison of CD to UC. Table 26 contains data for comparison of IBS to UC. Table 27 contains data for comparison of IBS to CD. Table 28 contains data for comparison of HC to IBS.

TABLE 23
RAF Analysis for HC vs. UC (G > T; A893S)

| | | |
|---|---|---|
| P value | 5.25E−05 | (95% Confidence) |

| | | | |
|---|---|---|---|
| Odds Ratio | 1.941176 | 1.405255 | 2.681483 |

TABLE 24
RAF Analysis for HC vs. CD (G > T; A893S)

| | | |
|---|---|---|
| P value | 0.078882 | (95% Confidence) |

| | | | |
|---|---|---|---|
| Odds Ratio | 1.294118 | 0.970428 | 1.725774 |

TABLE 25
RAF Analysis for CD vs. UC (G > T; A893S)

| | | |
|---|---|---|
| P value | 0.006594 | (95% Confidence) |

| | | | |
|---|---|---|---|
| Odds Ratio | 1.5 | 1.118869 | 2.01096 |

TABLE 26
RAF Analysis for IBS vs. UC (G > T; A893S)

| | | |
|---|---|---|
| P value | 0.097193 | (95% Confidence) |

| | | | |
|---|---|---|---|
| Odds Ratio | 1.409639 | 0.938878 | 2.116441 |

TABLE 27
RAF Analysis for IBS vs. CD (G > T; A893S)

| | | |
|---|---|---|
| P value | 0.762812 | (95% Confidence) |

| | | | |
|---|---|---|---|
| Odds Ratio | 0.943089 | 0.644574 | 1.379854 |

TABLE 28
RAF Analysis for HC vs. IBS (G > T; A893S)

| | | |
|---|---|---|
| P value | 0.124187 | (95% Confidence) |

| | | | |
|---|---|---|---|
| Odds Ratio | 0.728751 | 0.486509 | 1.091609 |

Tables 23 and 25 show that the MDR1 (A893S) rs2032582 variant allele was significantly associated with UC compared to HC or CD. As such, determining the presence or absence of the MDR1 (rs2032582) variant allele in accordance with the present invention is particularly useful for diagnosing UC, e.g., by identifying patients as having UC versus healthy control patients and/or patients with CD.

### Example 9

### Detection of ATG16L1 (T300A) rs2241880

The ATG16L1 gene is located on Chromosome 12. This rs2241880 is a mis-sense mutation consisting of a transition A to G with a codon change ACT to GCT. The rs2241880 SNP is located at position 1155 on the transcript NM_{—}030803.6 (SEQ ID NO:31). The transition leads to a AA change T300A on the protein ID NP_{—}110430.5 (SEQ ID NO:32).

For the detection of the rs2241880 SNP, the ABI TaqMAN assay was used (Applied Biosystems, USA). The ABI assay ID was C_{—}9095577_{—}20 (available from Applied Biosystems, USA). The following context sequence was used for the TaqMan assay [VIC/FAM]: CCCAGTCCCCCAGGACAATGTGGAT[A/G]CTCATCCTGGTTCTGGTAAAGAAGT (SEQ ID NO:42). The notation [A/G] represents the location of the rs2241880 SNP. The VIC labeled version of probe contains the A allele and the FAM labeled version of the probe contains the G allele.

For assay result interpretation for the rs2241880 SNP analysis, a FAM/FAM (G/G) signal was called as homozygous mutant; a VIC/VIC (A/A) signal was called as homozygous wild-type; and a VIC/FAM signal was called as heterozygous mutant. These results are shown in Table 29.

TABLE 29
ATG16L1 T281A/T300A (C 9095577 20; rs2241880)

| Diagnosis | Count | VIC (A) | VIC % | BOTH | BOTH % | FAM (G) | FAM % |
|---|---|---|---|---|---|---|---|
| IBD CROHN'S DISEASE | 420 | 82 | 19.5% | 195 | 46.4% | 143 | 34.0% |
| IBD ULCERATIVE COLITIS | 267 | 61 | 22.8% | 107 | 40.1% | 99 | 37.1% |
| HC/HEALTHY CONTROL/NORMAL | 414 | 145 | 35.0% | 165 | 39.9% | 104 | 25.1% |
| IBS GI CONTROL | 175 | 71 | 40.6% | 51 | 29.1% | 53 | 30.3% |

The results in the following Tables 30-35 represent the Risk Allele Factor (RAF) for the rs2241880 SNP. The risk allele is G. The p values were calculated using the frequency of both alleles after the heterozygous mutant values were split and equally redistributed in both homozygous wild-type and homozygous mutant genotypes. The different populations (Crohn's Disease (CD), Ulcerative Colitis (UC), Healthy Control (HC), and IBS GI Control (IBS)) were then compared between each other. Table 30 contains data for comparison of HC to UC. Table 31 contains data for comparison of HC to CD. Table 32 contains data for comparison of CD to UC. Table 33 contains data for comparison of IBS to UC. Table 34 contains data for comparison of IBS to CD. Table 35 contains data for comparison of IBS to HC.

TABLE 30
RAF Analysis for HC vs. UC

| | | |
|---|---|---|
| P value | 0.00223 | (95% Confidence) |

| | | | |
|---|---|---|---|
| Odds Ratio | 1.620155 | 1.188117 | 2.209297 |

TABLE 31
RAF Analysis for HC vs. CD

| | | |
|---|---|---|
| P value | 0.000173 | (95% Confidence) |

| | | | |
|---|---|---|---|
| Odds Ratio | 1.687831 | 1.283397 | 2.219713 |

TABLE 32
RAF Analysis for CD vs. UC

| | | |
|---|---|---|
| P value | 0.795992 | (95% Confidence) |

| | | | |
|---|---|---|---|
| Odds Ratio | 0.959904 | 0.703868 | 1.309075 |

TABLE 33
RAF Analysis for IBS vs. UC

| | | |
|---|---|---|
| P value | 0.013508 | (95% Confidence) |

| | | | |
|---|---|---|---|
| Odds Ratio | 1.620155 | 1.103622 | 2.378442 |

TABLE 34
RAF Analysis for IBS vs. CD

| | | |
|---|---|---|
| P value | 0.007497 | (95% Confidence) |

| | | | |
|---|---|---|---|
| Odds Ratio | 1.620155 | 1.136029 | 2.310595 |

TABLE 35
RAF Analysis for IBS vs. HC

| | | |
|---|---|---|
| P value | 1 | (95% Confidence) |

| | | | |
|---|---|---|---|
| Odds Ratio | 1 | 0.701014 | 1.426506 |

Tables 30 and 33 show that the ATG16L1 (T300A) rs2241880 variant allele was significantly associated with UC compared to HC or IBS. Tables 31 and 34 show that the ATG16L1 (T300A) rs2241880 variant allele was significantly associated with CD compared to HC or IBS. As such, determining the presence or absence of the ATG16L1 (rs2241880) variant allele in accordance with the present invention is particularly useful for diagnosing UC, e.g., by identifying patients as having UC versus healthy control patients and/or IBS GI control patients.

It is understood that the examples and embodiments described herein are for illustrative purposes only and that various modifications or changes in light thereof will be suggested to persons skilled in the art and are to be included within the spirit and purview of this application and scope of the appended claims. All publications including but not limited to patents, patent applications, journal articles, Genbank Accession Nos., and GeneID Nos. cited herein are hereby incorporated by reference in their entirety for all purposes.

1. A method for diagnosing ulcerative colitis (UC) in an individual diagnosed with inflammatory bowel disease (IBD), said method comprising:
(i) analyzing a biological sample obtained from said individual to determine the presence or absence of a variant allele in a gene selected from the group consisting of GLI1, MDR1, ATG16L1, and a combination thereof in said sample; and
(ii) associating the presence of said variant allele with a diagnosis of UC.

2. The method of claim 1, wherein said variant allele comprises GLI1 (rs2228224), GLI1 (rs2228226), or a combination thereof.

3. The method of claim 1, wherein said variant allele comprises MDR1 (rs2032582).

4. The method of claim 1, wherein said variant allele comprises ATG16L1 (rs2241880).

5. The method of claim 1, wherein said variant allele comprises one or more alleles selected from the group consisting of GLI1 (rs2228224), GLI1 (rs2228226), MDR1 (rs2032582), and ATG16L1 (rs2241880).

6. The method of claim 1, wherein said method improves the diagnosis of UC compared to detecting ANCA and/or pANCA.

7. The method of claim 1, comprising an additional step of analyzing said biological sample for the presence or level of a serological marker, wherein detection of the presence or level of said serological marker in conjunction with the presence of one or more variant alleles further improves the diagnosis of UC.

8. The method of claim 7, wherein said serological marker is selected from the group consisting of an anti-neutrophil antibody, an anti-Saccharomyces cerevisiae antibody, an antimicrobial antibody, an acute phase protein, an apolipoprotein, a defensin, a growth factor, a cytokine, a cadherin, and a combination thereof.

9. The method of claim 8, wherein said anti-neutrophil antibody is selected from the group consisting of ANCA, pANCA, and a combination thereof.

10. The method of claim 8, wherein said anti-Saccharomyces cerevisiae antibody is selected from the group consisting of anti-Saccharomyces cerevisiae immunoglobulin A (ASCA-IgA), anti-Saccharomyces cerevisiae immunoglobulin G (ASCA-IgG), and a combination thereof.

11. The method of claim 8, wherein said antimicrobial antibody is selected from the group consisting of an anti-outer membrane protein C (anti-OmpC) antibody, an anti-I2 antibody, an anti-flagellin antibody, and a combination thereof.

12. The method of claim 7, wherein said serological marker is selected from the group consisting of ANCA, pANCA, ASCA-IgA, ASCA-IgG, anti-OmpC antibody, anti-CBir-1 antibody, anti-I2 antibody, and a combination thereof.

13. The method of claim 1, wherein said individual has symptoms of UC.

14. The method of claim 13, wherein the symptoms of UC are selected from the group consisting of rectal inflammation, rectal bleeding, rectal pain, diarrhea, abdominal cramps, abdominal pain, fatigue, weight loss, fever, colon rupture and combinations thereof.

15. The method of claim 1, wherein said biological sample is selected from the group consisting of blood, tissue, saliva, cheek cells, hair, fluid, plasma, serum, cerebrospinal fluid, buccal swabs, mucus, urine, stools, spermatozoids, vaginal secretions, lymph, amniotic fluid, pleural liquid, tears, and combinations thereof.

16. The method of claim 1, wherein the presence or absence of said variant allele is determined using an assay selected from the group consisting of electrophoretic analysis assays, restriction length polymorphism analysis assays, sequence analysis assays, hybridization analysis assays, PCR analysis assays, allele-specific hybridization, oligonucleotide ligation allele-specific elongation/ligation, allele-specific amplification, single-base extension, molecular inversion probe, invasive cleavage, selective termination, restriction length polymorphism, sequencing, single strand conformation polymorphism (SSCP), single strand chain polymorphism, mismatch-cleaving, denaturing gradient gel electrophoresis, and combinations thereof.

17. A method for differentiating between ulcerative colitis (UC) and Crohn's disease (CD) in an individual diagnosed with inflammatory bowel disease (IBD), said method comprising:
(i) analyzing a biological sample obtained from said individual to determine the presence or absence of a variant allele in a gene selected from the group consisting of GLI1, MDR1, and a combination thereof in said sample; and
(ii) associating the presence of said variant allele with a diagnosis of UC.

18. The method of claim 17, wherein said variant allele comprises GLI1 (rs2228224), GLI1 (rs2228226), or a combination thereof.

19. The method of claim 17, wherein said variant allele comprises MDR1 (rs2032582).

20. The method of claim 17, wherein said variant allele comprises one or more alleles selected from the group consisting of GLI1 (rs2228224), GLI1 (rs2228226), and MDR1 (rs2032582).

21. The method of claim 17, comprising an additional step of analyzing said biological sample for the presence or level of a serological marker.

22. The method of claim 17, wherein said biological sample is selected from the group consisting of blood, tissue, saliva, cheek cells, hair, fluid, plasma, serum, cerebrospinal fluid, buccal swabs, mucus, urine, stools, spermatozoids, vaginal secretions, lymph, amniotic fluid, pleural liquid, tears, and combinations thereof.

23. The method of claim 17, wherein the presence or absence of said variant allele is determined using an assay selected from the group consisting of electrophoretic analysis assays, restriction length polymorphism analysis assays, sequence analysis assays, hybridization analysis assays, PCR analysis assays, allele-specific hybridization, oligonucleotide ligation allele-specific elongation/ligation, allele-specific amplification, single-base extension, molecular inversion probe, invasive cleavage, selective termination, restriction length polymorphism, sequencing, single strand conformation polymorphism (SSCP), single strand chain polymorphism, mismatch-cleaving, denaturing gradient gel electrophoresis, and combinations thereof.

24. The method of claim 17, wherein said individual has symptoms of UC.

25. The method of claim 24, wherein the symptoms of UC are selected from the group consisting of rectal inflammation, rectal bleeding, rectal pain, diarrhea, abdominal cramps, abdominal pain, fatigue, weight loss, fever, colon rupture and combinations thereof.


SEQUENCE LISTING (49 sequences)

SEQ ID NO: 1 - protein, 212 aa, Homo sapiens
  interleukin 6 (IL-6) precursor, interferon beta 2 (IFNB2), HGF, HSF, BSF2
MNSFSTSAFGPVAFSLGLLLVLPAAFPAPVPPGEDSKDVAAPHRQPLTSSERIDKQIRYILDGISALRKETCNKSNMCESSKEALAENNLNLPKMAEKDGCFQSGFNEETCLVKIITGLLEFEVYLEYLQNRFESSEEQARAVQMSTKVLIQFLQKKAKNLDAITTPDPTTNASLLTKLQAQNQWLQDMTTHLILRSFKEFLQSSLRALRQM

SEQ ID NO: 2 - DNA, 1201 bp, Homo sapiens
  interferon beta 2 (IFNB2), HGF, HSF, BSF2 cDNA
aatattagagtctcaacccccaataaatataggactggagatgtctgaggctcattctgccctcgagcccaccgggaacgaaagagaagctctatctcccctccaggagcccagctatgaactccttctccacaagcgccttcggtccagttgccttctccctggggctgctcctggtgttgcctgctgccttccctgccccagtacccccaggagaagattccaaagatgtagccgccccacacagacagccactcacctcttcagaacgaattgacaaacaaattcggtacatcctcgacggcatctcagccctgagaaaggagacatgtaacaagagtaacatgtgtgaaagcagcaaagaggcactggcagaaaacaacctgaaccttccaaagatggctgaaaaagatggatgcttccaatctggattcaatgaggagacttgcctggtgaaaatcatcactggtcttttggagtttgaggtatacctagagtacctccagaacagatttgagagtagtgaggaacaagccagagctgtgcagatgagtacaaaagtcctgatccagttcctgcagaaaaaggcaaagaatctagatgcaataaccacccctgacccaaccacaaatgccagcctgctgacgaagctgcaggcacagaaccagtggctgcaggacatgacaactcatctcattctgcgcagctttaaggagttcctgcagtccagcctgagggctcttcggcaaatgtagcatgggcacctcagattgttgttgttaatgggcattccttcttctggtcagaaacctgtccactgggcacagaacttatgttgttctctatggagaactaaaagtatgagcgttaggacactattttaattatttttaatttattaatatttaaatatgtgaagctgagttaatttatgtaagtcatatttatatttttaagaagtaccacttgaaacattttatgtattagttttgaaataataatggaaagtggctatgcagtttgaatatcctttgtttcagagccagatcatttcttggaaagtgtaggcttacctcaaataaatggctaacttatacatatttttaaagaaatatttatattgtatttatataatgtataaatggtttttataccaataaatggcattttaaaaaattcagcaaaaaaaaaaaaaaaaaaa

SEQ ID NO: 3 - protein, 269 aa, Homo sapiens
  interleukin -1 beta (IL-1beta) proprotein, IL1F2
MAEVPELASEMMAYYSGNEDDLFFEADGPKQMKCSFQDLDLCPLDGGIQLRISDHHYSKGFRQAASVVVAMDKLRKMLVPCPQTFQENDLSTFFPFIFEEEPIFFDTWDNEAYVHDAPVRSLNCTLRDSQQKSLVMSGPYELKALHLQGQDMEQQVVFSMSFVQGEESNDKIPVALGLKEKNLYLSCVLKDDKPTLQLESVDPKNYPKKKMEKRFVFNKIEINNKLEFESAQFPNWYISTSQAENMPVFLGGTKGGQDITDFTMQFVSS

SEQ ID NO: 4 - DNA, 1498 bp, Homo sapiens
  interleukin -1 beta (IL-1beta) proprotein, IL1F2 cDNA
accaaacctcttcgaggcacaaggcacaacaggctgctctgggattctcttcagccaatcttcattgctcaagtgtctgaagcagccatggcagaagtacctgagctcgccagtgaaatgatggcttattacagtggcaatgaggatgacttgttctttgaagctgatggccctaaacagatgaagtgctccttccaggacctggacctctgccctctggatggcggcatccagctacgaatctccgaccaccactacagcaagggcttcaggcaggccgcgtcagttgttgtggccatggacaagctgaggaagatgctggttccctgcccacagaccttccaggagaatgacctgagcaccttctttcccttcatctttgaagaagaacctatcttcttcgacacatgggataacgaggcttatgtgcacgatgcacctgtacgatcactgaactgcacgctccgggactcacagcaaaaaagcttggtgatgtctggtccatatgaactgaaagctctccacctccagggacaggatatggagcaacaagtggtgttctccatgtcctttgtacaaggagaagaaagtaatgacaaaatacctgtggccttgggcctcaaggaaaagaatctgtacctgtcctgcgtgttgaaagatgataagcccactctacagctggagagtgtagatcccaaaaattacccaaagaagaagatggaaaagcgatttgtcttcaacaagatagaaatcaataacaagctggaatttgagtctgcccagttccccaactggtacatcagcacctctcaagcagaaaacatgcccgtcttcctgggagggaccaaaggcggccaggatataactgacttcaccatgcaatttgtgtcttcctaaagagagctgtacccagagagtcctgtgctgaatgtggactcaatccctagggctggcagaaagggaacagaaaggtttttgagtacggctatagcctggactttcctgttgtctacaccaatgcccaactgcctgccttagggtagtgctaagaggatctcctgtccatcagccaggacagtcagctctctcctttcagggccaatccccagcccttttgttgagccaggcctctctcacctctcctactcacttaaagcccgcctgacagaaaccacggccacatttggttctaagaaaccctctgtcattcgctcccacattctgatgagcaaccgcttccctatttatttatttatttgtttgtttgttttattcattggtctaatttattcaaagggggcaagaagtagcagtgtctgtaaaagagcctagtttttaatagctatggaatcaattcaatttggactggtgtgctctctttaaatcaagtcctttaattaagactgaaaatatataagctcagattatttaaatgggaatatttataaatgagcaaatatcatactgttcaatggttctgaaataaacttcactgaag

SEQ ID NO: 5 - protein, 249 aa, Homo sapiens
  tumor necrosis factor (TNF)-related weak inducer of apoptosis (TWEAK), tumor necrosis factor ligand superfamily memmber 12 (TNFSF12), APO3 ligand (APO3L), CD255, DR3 ligand, growth factor-inducible 14 (Fn14) ligand, UNQ181/ PRO207
MAARRSQRRRGRRGEPGTALLVPLALGLGLALACLGLLLAVVSLGSRASLSAQEPAQEELVAEEDQDPSELNPQTEESQDPAPFLNRLVRPRRSAPKGRKTRARRAIAAHYEVHPRPGQDGAQAGVDGTVSGWEEARINSSSPLRYNRQIGEFIVTRAGLYYLYCQVHFDEGKAVYLKLDLLVDGVLALRCLEEFSATAASSLGPQLRLCQVSGLLALRPGSSLRIRTLPWAHLKAAPFLTYFGLFQVH

SEQ ID NO: 6 - DNA, 1407 bp, Homo sapiens
  tumor necrosis factor (TNF)-related weak inducer of apoptosis (TWEAK), tumor necrosis factor ligand superfamily memmber 12 (TNFSF12), APO3 ligand (APO3L), CD255, DR3 ligand, growth factor-inducible 14 (Fn14) ligand, UNQ181/PRO207 cDNA
ctctccccggcccgatccgcccgccggctccccctcccccgatccctcgggtcccgggatgggggggcggtgaggcaggcacagccccccgcccccatggccgcccgtcggagccagaggcggagggggcgccggggggagccgggcaccgccctgctggtcccgctcgcgctgggcctgggcctggcgctggcctgcctcggcctcctgctggccgtggtcagtttggggagccgggcatcgctgtccgcccaggagcctgcccaggaggagctggtggcagaggaggaccaggacccgtcggaactgaatccccagacagaagaaagccaggatcctgcgcctttcctgaaccgactagttcggcctcgcagaagtgcacctaaaggccggaaaacacgggctcgaagagcgatcgcagcccattatgaagttcatccacgacctggacaggacggagcgcaggcaggtgtggacgggacagtgagtggctgggaggaagccagaatcaacagctccagccctctgcgctacaaccgccagatcggggagtttatagtcacccgggctgggctctactacctgtactgtcaggtgcactttgatgaggggaaggctgtctacctgaagctggacttgctggtggatggtgtgctggccctgcgctgcctggaggaattctcagccactgcggcgagttccctcgggccccagctccgcctctgccaggtgtctgggctgttggccctgcggccagggtcctccctgcggatccgcaccctcccctgggcccatctcaaggctgcccccttcctcacctacttcggactcttccaggttcactgaggggccctggtctccccgcagtcgtcccaggctgccggctcccctcgacagctctctgggcacccggtcccctctgccccaccctcagccgctctttgctccagacctgcccctccctctagaggctgcctgggcctgttcacgtgttttccatcccacataaatacagtattcccactcttatcttacaactcccccaccgcccactctccacctcactagctccccaatccctgaccctttgaggcccccagtgatctcgactcccccctggccacagacccccagggcattgtgttcactgtactctgtgggcaaggatgggtccagaagaccccacttcaggcactaagaggggctggacctggcggcaggaagccaaagagactgggcctaggccaggagttcccaaatgtgaggggcgagaaacaagacaagctcctcccttgagaattccctgtggatttttaaaacagatattatttttattattattgtgacaaaatgttgataaatggatattaaatagaataagtcataaaaaaaaaaaaaaaaaaaaaa

SEQ ID NO: 7 - protein, 1207 aa, Homo sapiens
  epidermal growth factor (EGF), beta-urogastrone (URG), HOMG4
MLLTLIILLPVVSKFSFVSLSAPQHWSCPEGTLAGNGNSTCVGPAPFLIFSHGNSIFRIDTEGTNYEQLVVDAGVSVIMDFHYNEKRIYWVDLERQLLQRVFLNGSRQERVCNIEKNVSGMAINWINEEVIWSNQQEGIITVTDMKGNNSHILLSALKYPANVAVDPVERFIFWSSEVAGSLYRADLDGVGVKALLETSEKITAVSLDVLDKRLFWIQYNREGSNSLICSCDYDGGSVHISKHPTQHNLFAMSLFGDRIFYSTWKMKTIWIANKHTGKDMVRINLHSSFVPLGELKVVHPLAQPKAEDDTWEPEQKLCKLRKGNCSSTVCGQDLQSHLCMCAEGYALSRDRKYCEDVNECAFWNHGCTLGCKNTPGSYYCTCPVGFVLLPDGKRCHQLVSCPRNVSECSHDCVLTSEGPLCFCPEGSVLERDGKTCSGCSSPDNGGCSQLCVPLSPVSWECDCFPGYDLQLDEKSCAASGPQPFLLFANSQDIRHMHFDGTDYGTLLSQQMGMVYALDHDPVENKIYFAHTALKWIERANMDGSQRERLIEEGVDVPEGLAVDWIGRRFYWTDRGKSLIGRSDLNGKRSKIITKENISQPRGIAVHPMAKRLFWTDTGINPRIESSSLQGLGRLVIASSDLIWPSGITIDFLTDKLYWCDAKQSVIEMANLDGSKRRRLTQNDVGHPFAVAVFEDYVWFSDWAMPSVMRVNKRTGKDRVRLQGSMLKPSSLVVVHPLAKPGADPCLYQNGGCEHICKKRLGTAWCSCREGFMKASDGKTCLALDGHQLLAGGEVDLKNQVTPLDILSKTRVSEDNITESQHMLVAEIMVSDQDDCAPVGCSMYARCISEGEDATCQCLKGFAGDGKLCSDIDECEMGVPVCPPASSKCINTEGGYVCRCSEGYQGDGIHCLDIDECQLGEHSCGENASCTNTEGGYTCMCAGRLSEPGLICPDSTPPPHLREDDHHYSVRNSDSECPLSHDGYCLHDGVCMYIEALDKYACNCVVGYIGERCQYRDLKWWELRHAGHGQQQKVIVVAVCVVVLVMLLLLSLWGAHYYRTQKLLSKNPKNPYEESSRDVRSRRPADTEDGMSSCPQPWFVVIKEHQDLKNGGQPVAGEDGQAADGSMQPTSWRQEPQLCGMGTEQGCWIPVSSDKGSCPQVMERSFHMPSYGTQTLEGGVEKPHSLLSANPLWQQRALDPPHQMELTQ

SEQ ID NO: 8 - DNA, 4913 bp, Homo sapiens
  epidermal growth factor (EGF), beta-urogastrone (URG), HOMG4 cDNA
aaaaagagaaactgttgggagaggaatcgtatctccatatttcttctttcagccccaatccaagggttgtagctggaactttccatcagttcttcctttctttttcctctctaagcctttgccttgctctgtcacagtgaagtcagccagagcagggctgttaaactctgtgaaatttgtcataagggtgtcaggtatttcttactggcttccaaagaaacatagataaagaaatctttcctgtggcttcccttggcaggctgcattcagaaggtctctcagttgaagaaagagcttggaggacaacagcacaacaggagagtaaaagatgccccagggctgaggcctccgctcaggcagccgcatctggggtcaatcatactcaccttgcccgggccatgctccagcaaaatcaagctgttttcttttgaaagttcaaactcatcaagattatgctgctcactcttatcattctgttgccagtagtttcaaaatttagttttgttagtctctcagcaccgcagcactggagctgtcctgaaggtactctcgcaggaaatgggaattctacttgtgtgggtcctgcacccttcttaattttctcccatggaaatagtatctttaggattgacacagaaggaaccaattatgagcaattggtggtggatgctggtgtctcagtgatcatggattttcattataatgagaaaagaatctattgggtggatttagaaagacaacttttgcaaagagtttttctgaatgggtcaaggcaagagagagtatgtaatatagagaaaaatgtttctggaatggcaataaattggataaatgaagaagttatttggtcaaatcaacaggaaggaatcattacagtaacagatatgaaaggaaataattcccacattcttttaagtgctttaaaatatcctgcaaatgtagcagttgatccagtagaaaggtttatattttggtcttcagaggtggctggaagcctttatagagcagatctcgatggtgtgggagtgaaggctctgttggagacatcagagaaaataacagctgtgtcattggatgtgcttgataagcggctgttttggattcagtacaacagagaaggaagcaattctcttatttgctcctgtgattatgatggaggttctgtccacattagtaaacatccaacacagcataatttgtttgcaatgtccctttttggtgaccgtatcttctattcaacatggaaaatgaagacaatttggatagccaacaaacacactggaaaggacatggttagaattaacctccattcatcatttgtaccacttggtgaactgaaagtagtgcatccacttgcacaacccaaggcagaagatgacacttgggagcctgagcagaaactttgcaaattgaggaaaggaaactgcagcagcactgtgtgtgggcaagacctccagtcacacttgtgcatgtgtgcagagggatacgccctaagtcgagaccggaagtactgtgaagatgttaatgaatgtgctttttggaatcatggctgtactcttgggtgtaaaaacacccctggatcctattactgcacgtgccctgtaggatttgttctgcttcctgatgggaaacgatgtcatcaacttgtttcctgtccacgcaatgtgtctgaatgcagccatgactgtgttctgacatcagaaggtcccttatgtttctgtcctgaaggctcagtgcttgagagagatgggaaaacatgtagcggttgttcctcacccgataatggtggatgtagccagctctgcgttcctcttagcccagtatcctgggaatgtgattgctttcctgggtatgacctacaactggatgaaaaaagctgtgcagcttcaggaccacaaccatttttgctgtttgccaattctcaagatattcgacacatgcattttgatggaacagactatggaactctgctcagccagcagatgggaatggtttatgccctagatcatgaccctgtggaaaataagatatactttgcccatacagccctgaagtggatagagagagctaatatggatggttcccagcgagaaaggcttattgaggaaggagtagatgtgccagaaggtcttgctgtggactggattggccgtagattctattggacagacagagggaaatctctgattggaaggagtgatttaaatgggaaacgttccaaaataatcactaaggagaacatctctcaaccacgaggaattgctgttcatccaatggccaagagattattctggactgatacagggattaatccacgaattgaaagttcttccctccaaggccttggccgtctggttatagccagctctgatctaatctggcccagtggaataacgattgacttcttaactgacaagttgtactggtgcgatgccaagcagtctgtgattgaaatggccaatctggatggttcaaaacgccgaagacttacccagaatgatgtaggtcacccatttgctgtagcagtgtttgaggattatgtgtggttctcagattgggctatgccatcagtaatgagagtaaacaagaggactggcaaagatagagtacgtctccaaggcagcatgctgaagccctcatcactggttgtggttcatccattggcaaaaccaggagcagatccctgcttatatcaaaacggaggctgtgaacatatttgcaaaaagaggcttggaactgcttggtgttcgtgtcgtgaaggttttatgaaagcctcagatgggaaaacgtgtctggctctggatggtcatcagctgttggcaggtggtgaagttgatctaaagaaccaagtaacaccattggacatcttgtccaagactagagtgtcagaagataacattacagaatctcaacacatgctagtggctgaaatcatggtgtcagatcaagatgactgtgctcctgtgggatgcagcatgtatgctcggtgtatttcagagggagaggatgccacatgtcagtgtttgaaaggatttgctggggatggaaaactatgttctgatatagatgaatgtgagatgggtgtcccagtgtgcccccctgcctcctccaagtgcatcaacaccgaaggtggttatgtctgccggtgctcagaaggctaccaaggagatgggattcactgtcttgatattgatgagtgccaactgggggagcacagctgtggagagaatgccagctgcacaaatacagagggaggctatacctgcatgtgtgctggacgcctgtctgaaccaggactgatttgccctgactctactccaccccctcacctcagggaagatgaccaccactattccgtaagaaatagtgactctgaatgtcccctgtcccacgatgggtactgcctccatgatggtgtgtgcatgtatattgaagcattggacaagtatgcatgcaactgtgttgttggctacatcggggagcgatgtcagtaccgagacctgaagtggtgggaactgcgccacgctggccacgggcagcagcagaaggtcatcgtggtggctgtctgcgtggtggtgcttgtcatgctgctcctcctgagcctgtggggggcccactactacaggactcagaagctgctatcgaaaaacccaaagaatccttatgaggagtcgagcagagatgtgaggagtcgcaggcctgctgacactgaggatgggatgtcctcttgccctcaaccttggtttgtggttataaaagaacaccaagacctcaagaatgggggtcaaccagtggctggtgaggatggccaggcagcagatgggtcaatgcaaccaacttcatggaggcaggagccccagttatgtggaatgggcacagagcaaggctgctggattccagtatccagtgataagggctcctgtccccaggtaatggagcgaagctttcatatgccctcctatgggacacagacccttgaagggggtgtcgagaagccccattctctcctatcagctaacccattatggcaacaaagggccctggacccaccacaccaaatggagctgactcagtgaaaactggaattaaaaggaaagtcaagaagaatgaactatgtcgatgcacagtatcttttctttcaaaagtagagcaaaactataggttttggttccacaatctctacgactaatcacctactcaatgcctggagacagatacgtagttgtgcttttgtttgctcttttaagcagtctcactgcagtcttatttccaagtaagagtactgggagaatcactaggtaacttattagaaacccaaattgggacaacagtgctttgtaaattgtgttgtcttcagcagtcaatacaaatagatttttgtttttgttgttcctgcagccccagaagaaattaggggttaaagcagacagtcacactggtttggtcagttacaaagtaatttctttgatctggacagaacatttatatcagtttcatgaaatgattggaatattacaataccgttaagatacagtgtaggcatttaactcctcattggcgtggtccatgctgatgattttgcaaaatgagttgtgatgaatcaatgaaaaatgtaatttagaaactgatttcttcagaattagatggcttattttttaaaatatttgaatgaaaacattttatttttaaaatattacacaggaggcttcggagtttcttagtcattactgtccttttcccctacagaattttccctcttggtgtgattgcacagaatttgtatgtattttcagttacaagattgtaagtaaattgcctgatttgttttcattatagacaacgatgaatttcttctaattatttaaataaaatcaccaaaaacataaaaaaaaaaaaaaaaaaaaaaaaaaaaa

SEQ ID NO: 9 - protein, 224 aa, Homo sapiens
  C-reactive protein (CRP), PTX1, MGC88244, MGC149895
MEKLLCFLVLTSLSHAFGQTDMSRKAFVFPKESDTSYVSLKAPLTKPLKAFTVCLHFYTELSSTRGYSIFSYATKRQDNEILIFWSKDIGYSFTVGGSEILFEVPEVTVAPVHICTSWESASGIVEFWVDGKPRVRKSLKKGYTVGAEASIILGQEQDSFGGNFEGSQSLVGDIGNVNMWDFVLSPDEINTIYLGGPFSPNVLNWRALKYEVQGEVFTKPQLWP

SEQ ID NO: 10 - DNA, 2024 bp, Homo sapiens
  C-reactive protein (CRP), PTX1, MGC88244, MGC149895 cDNA
aaggcaagagatctaggacttctagcccctgaactttcagccgaatacatcttttccaaaggagtgaattcaggcccttgtatcactggcagcaggacgtgaccatggagaagctgttgtgtttcttggtcttgaccagcctctctcatgcttttggccagacagacatgtcgaggaaggcttttgtgtttcccaaagagtcggatacttcctatgtatccctcaaagcaccgttaacgaagcctctcaaagccttcactgtgtgcctccacttctacacggaactgtcctcgacccgtgggtacagtattttctcgtatgccaccaagagacaagacaatgagattctcatattttggtctaaggatataggatacagttttacagtgggtgggtctgaaatattattcgaggttcctgaagtcacagtagctccagtacacatttgtacaagctgggagtccgcctcagggatcgtggagttctgggtagatgggaagcccagggtgaggaagagtctgaagaagggatacactgtgggggcagaagcaagcatcatcttggggcaggagcaggattccttcggtgggaactttgaaggaagccagtccctggtgggagacattggaaatgtgaacatgtgggactttgtgctgtcaccagatgagattaacaccatctatcttggcgggcccttcagtcctaatgtcctgaactggcgggcactgaagtatgaagtgcaaggcgaagtgttcaccaaaccccagctgtggccctgaggcccagctgtgggtcctgaaggtacctcccggttttttacaccgcatgggccccacgtctctgtctctggtacctcccgcttttttacactgcatggttcccacgtctctgtctctgggcctttgttcccctatatgcattgcaggcctgctccaccctcctcagcgcctgagaatggaggtaaagtgtctggtctgggagctcgttaactatgctgggaaacggtccaaaagaatcagaatttgaggtgttttgttttcatttttatttcaagttggacagatcttggagataatttcttacctcacatagatgagaaaactaacacccagaaaggagaaatgatgttataaaaaactcataaggcaagagctgagaaggaagcgctgatcttctatttaattccccacccatgacccccagaaagcaggagggcattgcccacattcacagggctcttcagtctcagaatcaggacactggccaggtgtctggtttgggtccagagtgctcatcatcatgtcatagaactgctgggcccaggtctcctgaaatgggaagcccagcaataccacgcagtccctccactttctcaaagcacactggaaaggccattagaattgccccagcagagcagatctgctttttttccagagcaaaatgaagcactaggtataaatatgttgttactgccaagaacttaaatgactggtttttgtttgcttgcagtgctttcttaattttatggctcttctgggaaactcctccccttttccacacgaaccttgtggggctgtgaattctttcttcatccccgcattcccaatatacccaggccacaagagtggacgtgaaccacagggtgtcctgtcagaggagcccatctcccatctccccagctccctatctggaggatagttggatagttacgtgttcctagcaggaccaactacagtcttcccaaggattgagttatggactttgggagtgagacatcttcttgctgctggatttccaagctgagaggacgtgaacctgggaccaccagtagccatcttgtttgccacatggagagagactgtgaggacagaagccaaactggaagtggaggagccaagggattgacaaacaacagagccttgaccacgtggagtctctgaatcagccttgtctggaaccagatctacacctggactgcccaggtctataagccaataaagcccctgtttacttgaaaaaaaaaa

SEQ ID NO: 11 - protein, 122 aa, Homo sapiens
  acute phase serum amyloid A (SAA), serum amyloid A1 (SAA1), PIG4, TP53I4, MGC111216
MKLLTGLVFCSLVLGVSSRSFFSFLGEAFDGARDMWRAYSDMREANYIGSDKYFHARGNYDAAKRGPGGAWAAEVISDARENIQRFFGHGAEDSLADQAANEWGRSGKDPNHFRPAGLPEKY

SEQ ID NO: 12 - DNA, 716 bp, Homo sapiens
  acute phase serum amyloid A (SAA), serum amyloid A1 (SAA1), PIG4, TP53I4, MGC111216 cDNA
aggctcagtataaatagcagccaccgctccctggcaggcagggacccgcagctcagctacagcacagatcaggtgaggagcacaccaaggagtgatttttaaaacttactctgttttctctttcccaacaagattatcatttcctttaaaaaaaatagttatcctggggcatacagccataccattctgaaggtgtcttatctcctctgatctagagagcaccatgaagcttctcacgggcctggttttctgctccttggtcctgggtgtcagcagccgaagcttcttttcgttccttggcgaggcttttgatggggctcgggacatgtggagagcctactctgacatgagagaagccaattacatcggctcagacaaatacttccatgctcgggggaactatgatgctgccaaaaggggacctgggggtgcctgggctgcagaagtgatcagcgatgccagagagaatatccagagattctttggccatggtgcggaggactcgctggctgatcaggctgccaatgaatggggcaggagtggcaaagaccccaatcacttccgacctgctggcctgcctgagaaatactgagcttcctcttcactctgctctcaggagatctggctgtgaggccctcagggcagggatacaaagcggggagagggtacacaatgggtatctaataaatacttaagaggtggaatttgtggaaact

SEQ ID NO: 13 - protein, 68 aa, Homo sapiens
  beta-defensin-1 (DEFB1, BD1, HBD1, DEFB-1), DEFB101, MGC51822
MRTSYLLLFTLCLLLSEMASGGNFLTGLGHRSDHYNCVSSGGQCLYSACPIFTKIQGTCYRGKAKCCK

SEQ ID NO: 14 - DNA, 484 bp, Homo sapiens
  beta-defensin-1 (DEFB1, BD1, HBD1, DEFB-1), DEFB101, MGC51822 cDNA
tcccttcagttccgtcgacgaggttgtgcaatccaccagtcttataaatacagtgacgctccagcctctggaagcctctgtcagctcagcctccaaaggagccagcgtctccccagttcctgaaatcctgggtgttgcctgccagtcgccatgagaacttcctaccttctgctgtttactctctgcttacttttgtctgagatggcctcaggtggtaactttctcacaggccttggccacagatctgatcattacaattgcgtcagcagtggagggcaatgtctctattctgcctgcccgatctttaccaaaattcaaggcacctgttacagagggaaggccaagtgctgcaagtgagctgggagtgaccagaagaaatgacgcagaagtgaaatgaactttttataagcattcttttaataaaggaaaattgcttttgaagtatacctcctttgggccaaaaaaaaaaaaaaaaaaaaaaaaa

SEQ ID NO: 15 - protein, 64 aa, Homo sapiens
  beta-defensin-2 (DEFB2, SAP1, HBD-2, DEFB-2), DEFB102, DEFB4
MRVLYLLFSFLFIFLMPLPGVFGGIGDPVTCLKSGAICHPVFCPRRYKQIGTCGLPGTKCCKKP

SEQ ID NO: 16 - DNA, 336 bp, Homo sapiens
  beta-defensin-2 (DEFB2, SAP1, HBD-2, DEFB-2), DEFB102, DEFB4 cDNA
agactcagctcctggtgaagctcccagccatcagccatgagggtcttgtatctcctcttctcgttcctcttcatattcctgatgcctcttccaggtgtttttggtggtataggcgatcctgttacctgccttaagagtggagccatatgtcatccagtcttttgccctagaaggtataaacaaattggcacctgtggtctccctggaacaaaatgctgcaaaaagccatgaggaggccaagaagctgctgtggctgatgcggattcagaaagggctccctcatcagagacgtgcgacatgtaaaccaaattaaactatggtgtccaaagatacgca

SEQ ID NO: 17 - protein, 882 aa, Homo sapiens
  E-cadherin (epithelial) (CDH1, CDHE, ECAD), UVO, LCAM, Arc-1, CD3244
MGPWSRSLSALLLLLQVSSWLCQEPEPCHPGFDAESYTFTVPRRHLERGRVLGRVNFEDCTGRQRTAYFSLDTRFKVGTDGVITVKRPLRFHNPQIHFLVYAWDSTYRKFSTKVTLNTVGHHHRPPPHQASVSGIQAELLTFPNSSPGLRRQKRDWVIPPISCPENEKGPFPKNLVQIKSNKDKEGKVFYSITGQGADTPPVGVFIIERETGWLKVTEPLDRERIATYTLFSHAVSSNGNAVEDPMEILITVTDQNDNKPEFTQEVFKGSVMEGALPGTSVMEVTATDADDDVNTYNAAIAYTILSQDPELPDKNMFTINRNTGVISVVTTGLDRESFPTYTLVVQAADLQGEGLSTTATAVITVTDTNDNPPIFNPTTYKGQVPENEANVVITTLKVTDADAPNTPAWEAVYTILNDDGGQFVVTTNPVNNDGILKTAKGLDFEAKQQYILHVAVTNVVPFEVSLTTSTATVTVDVLDVNEAPIFVPPEKRVEVSEDFGVGQEITSYTAQEPDTFMEQKITYRIWRDTANWLEINPDTGAISTRAELDREDFEHVKNSTYTALIIATDNGSPVATGTGTLLLILSDVNDNAPIPEPRTIFFCERNPKPQVINIIDADLPPNTSPFTAELTHGASANWTIQYNDPTQESIILKPKMALEVGDYKINLKLMDNQNKDQVTTLEVSVCDCEGAAGVCRKAQPVEAGLQIPAILGILGGILALLILILLLLLFLRRRAVVKEPLLPPEDDTRDNVYYYDEEGGGEEDQDFDLSQLHRGLDARPEVTRNDVAPTLMSVPRYLPRPANPDEIGNFIDENLKAADTDPTAPPYDSLLVFDYEGSGSEAASLSSLNSSESDKDQDYDYLNEWGNRFKKLADMYGGGEDD

SEQ ID NO: 18 - DNA, 4815 bp, Homo sapiens
  E-cadherin (epithelial) (CDH1, CDHE, ECAD), UVO, LCAM, Arc-1, CD3244 cDNA
agtggcgtcggaactgcaaagcacctgtgagcttgcggaagtcagttcagactccagcccgctccagcccggcccgacccgaccgcacccggcgcctgccctcgctcggcgtccccggccagccatgggcccttggagccgcagcctctcggcgctgctgctgctgctgcaggtctcctcttggctctgccaggagccggagccctgccaccctggctttgacgccgagagctacacgttcacggtgccccggcgccacctggagagaggccgcgtcctgggcagagtgaattttgaagattgcaccggtcgacaaaggacagcctatttttccctcgacacccgattcaaagtgggcacagatggtgtgattacagtcaaaaggcctctacggtttcataacccacagatccatttcttggtctacgcctgggactccacctacagaaagttttccaccaaagtcacgctgaatacagtggggcaccaccaccgccccccgccccatcaggcctccgtttctggaatccaagcagaattgctcacatttcccaactcctctcctggcctcagaagacagaagagagactgggttattcctcccatcagctgcccagaaaatgaaaaaggcccatttcctaaaaacctggttcagatcaaatccaacaaagacaaagaaggcaaggttttctacagcatcactggccaaggagctgacacaccccctgttggtgtctttattattgaaagagaaacaggatggctgaaggtgacagagcctctggatagagaacgcattgccacatacactctcttctctcacgctgtgtcatccaacgggaatgcagttgaggatccaatggagattttgatcacggtaaccgatcagaatgacaacaagcccgaattcacccaggaggtctttaaggggtctgtcatggaaggtgctcttccaggaacctctgtgatggaggtcacagccacagacgcggacgatgatgtgaacacctacaatgccgccatcgcttacaccatcctcagccaagatcctgagctccctgacaaaaatatgttcaccattaacaggaacacaggagtcatcagtgtggtcaccactgggctggaccgagagagtttccctacgtataccctggtggttcaagctgctgaccttcaaggtgaggggttaagcacaacagcaacagctgtgatcacagtcactgacaccaacgataatcctccgatcttcaatcccaccacgtacaagggtcaggtgcctgagaacgaggctaacgtcgtaatcaccacactgaaagtgactgatgctgatgcccccaataccccagcgtgggaggctgtatacaccatattgaatgatgatggtggacaatttgtcgtcaccacaaatccagtgaacaacgatggcattttgaaaacagcaaagggcttggattttgaggccaagcagcagtacattctacacgtagcagtgacgaatgtggtaccttttgaggtctctctcaccacctccacagccaccgtcaccgtggatgtgctggatgtgaatgaagcccccatctttgtgcctcctgaaaagagagtggaagtgtccgaggactttggcgtgggccaggaaatcacatcctacactgcccaggagccagacacatttatggaacagaaaataacatatcggatttggagagacactgccaactggctggagattaatccggacactggtgccatttccactcgggctgagctggacagggaggattttgagcacgtgaagaacagcacgtacacagccctaatcatagctacagacaatggttctccagttgctactggaacagggacacttctgctgatcctgtctgatgtgaatgacaacgcccccataccagaacctcgaactatattcttctgtgagaggaatccaaagcctcaggtcataaacatcattgatgcagaccttcctcccaatacatctcccttcacagcagaactaacacacggggcgagtgccaactggaccattcagtacaacgacccaacccaagaatctatcattttgaagccaaagatggccttagaggtgggtgactacaaaatcaatctcaagctcatggataaccagaataaagaccaagtgaccaccttagaggtcagcgtgtgtgactgtgaaggggccgctggcgtctgtaggaaggcacagcctgtcgaagcaggattgcaaattcctgccattctggggattcttggaggaattcttgctttgctaattctgattctgctgctcttgctgtttcttcggaggagagcggtggtcaaagagcccttactgcccccagaggatgacacccgggacaacgtttattactatgatgaagaaggaggcggagaagaggaccaggactttgacttgagccagctgcacaggggcctggacgctcggcctgaagtgactcgtaacgacgttgcaccaaccctcatgagtgtcccccggtatcttccccgccctgccaatcccgatgaaattggaaattttattgatgaaaatctgaaagcggctgatactgaccccacagccccgccttatgattctctgctcgtgtttgactatgaaggaagcggttccgaagctgctagtctgagctccctgaactcctcagagtcagacaaagaccaggactatgactacttgaacgaatggggcaatcgcttcaagaagctggctgacatgtacggaggcggcgaggacgactaggggactcgagagaggcgggccccagacccatgtgctgggaaatgcagaaatcacgttgctggtggtttttcagctcccttcccttgagatgagtttctggggaaaaaaaagagactggttagtgatgcagttagtatagctttatactctctccactttatagctctaataagtttgtgttagaaaagtttcgacttatttcttaaagcttttttttttttcccatcactctttacatggtggtgatgtccaaaagatacccaaattttaatattccagaagaacaactttagcatcagaaggttcacccagcaccttgcagattttcttaaggaattttgtctcacttttaaaaagaaggggagaagtcagctactctagttctgttgttttgtgtatataattttttaaaaaaaatttgtgtgcttctgctcattactacactggtgtgtccctctgccttttttttttttttaagacagggtctcattctatcggccaggctggagtgcagtggtgcaatcacagctcactgcagccttgtcctcccaggctcaagctatccttgcacctcagcctcccaagtagctgggaccacaggcatgcaccactacgcatgactaattttttaaatatttgagacggggtctccctgtgttacccaggctggtctcaaactcctgggctcaagtgatcctcccatcttggcctcccagagtattgggattacagacatgagccactgcacctgcccagctccccaactccctgccattttttaagagacagtttcgctccatcgcccaggcctgggatgcagtgatgtgatcatagctcactgtaacctcaaactctggggctcaagcagttctcccaccagcctcctttttatttttttgtacagatggggtcttgctatgttgcccaagctggtcttaaactcctggcctcaagcaatccttctgccttggccccccaaagtgctgggattgtgggcatgagctgctgtgcccagcctccatgttttaatatcaactctcactcctgaattcagttgctttgcccaagataggagttctctgatgcagaaattattgggctcttttagggtaagaagtttgtgtctttgtctggccacatcttgactaggtattgtctactctgaagacctttaatggcttccctctttcatctcctgagtatgtaacttgcaatgggcagctatccagtgacttgttctgagtaagtgtgttcattaatgtttatttagctctgaagcaagagtgatatactccaggacttagaatagtgcctaaagtgctgcagccaaagacagagcggaactatgaaaagtgggcttggagatggcaggagagcttgtcattgagcctggcaatttagcaaactgatgctgaggatgattgaggtgggtctacctcatctctgaaaattctggaaggaatggaggagtctcaacatgtgtttctgacacaagatccgtggtttgtactcaaagcccagaatccccaagtgcctgcttttgatgatgtctacagaaaatgctggctgagctgaacacatttgcccaattccaggtgtgcacagaaaaccgagaatattcaaaattccaaatttttttcttaggagcaagaagaaaatgtggccctaaagggggttagttgaggggtagggggtagtgaggatcttgatttggatctctttttatttaaatgtgaatttcaacttttgacaatcaaagaaaagacttttgttgaaatagctttactgtttctcaagtgttttggagaaaaaaatcaaccctgcaatcactttttggaattgtcttgatttttcggcagttcaagctatatcgaatatagttctgtgtagagaatgtcactgtagttttgagtgtatacatgtgtgggtgctgataattgtgtattttctttgggggtggaaaaggaaaacaattcaagctgagaaaagtattctcaaagatgcatttttataaattttattaaacaattttgttaaaccat

SEQ ID NO: 19 - DNA, 3249 bp, Homo sapiens
  intercellular adhesion molecule 1 (ICAM1) precursor cDNA
caagcttagcctggccgggaaacgggaggcgtggaggccgggagcagcccccggggtcatcgccctgccaccgccgcccgattgctttagcttggaaattccggagctgaagcggccagcgagggaggatgaccctctcggcccgggcaccctgtcagtccggaaataactgcagcatttgttccggaggggaaggcgcgaggtttccgggaaagcagcaccgccccttggcccccaggtggctagcgctataaaggatcacgcgccccagtcgacgctgagctcctctgctactcagagttgcaacctcagcctcgctatggctcccagcagcccccggcccgcgctgcccgcactcctggtcctgctcggggctctgttcccaggacctggcaatgcccagacatctgtgtccccctcaaaagtcatcctgccccggggaggctccgtgctggtgacatgcagcacctcctgtgaccagcccaagttgttgggcatagagaccccgttgcctaaaaaggagttgctcctgcctgggaacaaccggaaggtgtatgaactgagcaatgtgcaagaagatagccaaccaatgtgctattcaaactgccctgatgggcagtcaacagctaaaaccttcctcaccgtgtactggactccagaacgggtggaactggcacccctcccctcttggcagccagtgggcaagaaccttaccctacgctgccaggtggagggtggggcaccccgggccaacctcaccgtggtgctgctccgtggggagaaggagctgaaacgggagccagctgtgggggagcccgctgaggtcacgaccacggtgctggtgaggagagatcaccatggagccaatttctcgtgccgcactgaactggacctgcggccccaagggctggagctgtttgagaacacctcggccccctaccagctccagacctttgtcctgccagcgactcccccacaacttgtcagcccccgggtcctagaggtggacacgcaggggaccgtggtctgttccctggacgggctgttcccagtctcggaggcccaggtccacctggcactgggggaccagaggttgaaccccacagtcacctatggcaacgactccttctcggccaaggcctcagtcagtgtgaccgcagaggacgagggcacccagcggctgacgtgtgcagtaatactggggaaccagagccaggagacactgcagacagtgaccatctacagctttccggcgcccaacgtgattctgacgaagccagaggtctcagaagggaccgaggtgacagtgaagtgtgaggcccaccctagagccaaggtgacgctgaatggggttccagcccagccactgggcccgagggcccagctcctgctgaaggccaccccagaggacaacgggcgcagcttctcctgctctgcaaccctggaggtggccggccagcttatacacaagaaccagacccgggagcttcgtgtcctgtatggcccccgactggacgagagggattgtccgggaaactggacgtggccagaaaattcccagcagactccaatgtgccaggcttgggggaacccattgcccgagctcaagtgtctaaaggatggcactttcccactgcccatcggggaatcagtgactgtcactcgagatcttgagggcacctacctctgtcgggccaggagcactcaaggggaggtcacccgcaaggtgaccgtgaatgtgctctccccccggtatgagattgtcatcatcactgtggtagcagccgcagtcataatgggcactgcaggcctcagcacgtacctctataaccgccagcggaagatcaagaaatacagactacaacaggcccaaaaagggacccccatgaaaccgaacacacaagccacgcctccctgaacctatcccgggacagggcctcttcctcggccttcccatattggtggcagtggtgccacactgaacagagtggaagacatatgccatgcagctacacctaccggccctgggacgccggaggacagggcattgtcctcagtcagatacaacagcatttggggccatggtacctgcacacctaaaacactaggccacgcatctgatctgtagtcacatgactaagccaagaggaaggagcaagactcaagacatgattgatggatgttaaagtctagcctgatgagaggggaagtggtgggggagacatagccccaccatgaggacatacaactgggaaatactgaaacttgctgcctattgggtatgctgaggccccacagacttacagaagaagtggccctccatagacatgtgtagcatcaaaacacaaaggcccacacttcctgacggatgccagcttgggcactgctgtctactgaccccaacccttgatgatatgtatttattcatttgttattttaccagctatttattgagtgtcttttatgtaggctaaatgaacataggtctctggcctcacggagctcccagtcctaatcacattcaaggtcaccaggtacagttgtacaggttgtacactgcaggagagtgcctggcaaaaagatcaaatggggctgggacttctcattggccaacctgcctttccccagaaggagtgatttttctatcggcacaaaagcactatatggactggtaatggttacaggttcagagattacccagtgaggccttattcctcccttccccccaaaactgacacctttgttagccacctccccacccacatacatttctgccagtgttcacaatgacactcagcggtcatgtctggacatgagtgcccagggaatatgcccaagctatgccttgtcctcttgtcctgtttgcatttcactgggagcttgcactatgcagctccagtttcctgcagtgatcagggtcctgcaagcagtggggaagggggccaaggtattggaggactccctcccagctttggaagcctcatccgcgtgtgtgtgtgtgtgtatgtgtagacaagctctcgctctgtcacccaggctggagtgcagtggtgcaatcatggttcactgcagtcttgaccttttgggctcaagtgatcctcccacctcagcctcctgagtagctgggaccataggctcacaacaccacacctggcaaatttgattttttttttttttccagagacggggtctcgcaacattgcccagacttcctttgtgttagttaataaagctttctcaactgccaaa

SEQ ID NO: 20 - protein, 532 aa, Homo sapiens
  intercellular adhesion molecule 1 (ICAM1) precursor
MAPSSPRPALPALLVLLGALFPGPGNAQTSVSPSKVILPRGGSVLVTCSTSCDQPKLLGIETPLPKKELLLPGNNRKVYELSNVQEDSQPMCYSNCPDGQSTAKTFLTVYWTPERVELAPLPSWQPVGKNLTLRCQVEGGAPRANLTVVLLRGEKELKREPAVGEPAEVTTTVLVRRDHHGANFSCRTELDLRPQGLELFENTSAPYQLQTFVLPATPPQLVSPRVLEVDTQGTVVCSLDGLFPVSEAQVHLALGDQRLNPTVTYGNDSFSAKASVSVTAEDEGTQRLTCAVILGNQSQETLQTVTIYSFPAPNVILTKPEVSEGTEVTVKCEAHPRAKVTLNGVPAQPLGPRAQLLLKATPEDNGRSFSCSATLEVAGQLIHKNQTRELRVLYGPRLDERDCPGNWTWPENSQQTPMCQAWGNPLPELKCLKDGTFPLPIGESVTVTRDLEGTYLCRARSTQGEVTRKVTVNVLSPRYEIVIITVVAAAVIMGTAGLSTYLYNRQRKIKKYRLQQAQKGTPMKPNTQATPP

SEQ ID NO: 21 - DNA, 3119 bp, Homo sapiens
  vascular cell adhesion molecule 1 (VCAM1), transcript variant 1 cDNA
cgcggtatctgcatcgggcctcactggcttcaggagctgaataccctcccaggcacacacaggtgggacacaaataagggttttggaaccactattttctcatcacgacagcaacttaaaatgcctgggaagatggtcgtgatccttggagcctcaaatatactttggataatgtttgcagcttctcaagcttttaaaatcgagaccaccccagaatctagatatcttgctcagattggtgactccgtctcattgacttgcagcaccacaggctgtgagtccccatttttctcttggagaacccagatagatagtccactgaatgggaaggtgacgaatgaggggaccacatctacgctgacaatgaatcctgttagttttgggaacgaacactcttacctgtgcacagcaacttgtgaatctaggaaattggaaaaaggaatccaggtggagatctactcttttcctaaggatccagagattcatttgagtggccctctggaggctgggaagccgatcacagtcaagtgttcagttgctgatgtatacccatttgacaggctggagatagacttactgaaaggagatcatctcatgaagagtcaggaatttctggaggatgcagacaggaagtccctggaaaccaagagtttggaagtaacctttactcctgtcattgaggatattggaaaagttcttgtttgccgagctaaattacacattgatgaaatggattctgtgcccacagtaaggcaggctgtaaaagaattgcaagtctacatatcacccaagaatacagttatttctgtgaatccatccacaaagctgcaagaaggtggctctgtgaccatgacctgttccagcgagggtctaccagctccagagattttctggagtaagaaattagataatgggaatctacagcacctttctggaaatgcaactctcaccttaattgctatgaggatggaagattctggaatttatgtgtgtgaaggagttaatttgattgggaaaaacagaaaagaggtggaattaattgttcaagagaaaccatttactgttgagatctcccctggaccccggattgctgctcagattggagactcagtcatgttgacatgtagtgtcatgggctgtgaatccccatctttctcctggagaacccagatagacagccctctgagcgggaaggtgaggagtgaggggaccaattccacgctgaccctgagccctgtgagttttgagaacgaacactcttatctgtgcacagtgacttgtggacataagaaactggaaaagggaatccaggtggagctctactcattccctagagatccagaaatcgagatgagtggtggcctcgtgaatgggagctctgtcactgtaagctgcaaggttcctagcgtgtacccccttgaccggctggagattgaattacttaagggggagactattctggagaatatagagtttttggaggatacggatatgaaatctctagagaacaaaagtttggaaatgaccttcatccctaccattgaagatactggaaaagctcttgtttgtcaggctaagttacatattgatgacatggaattcgaacccaaacaaaggcagagtacgcaaacactttatgtcaatgttgcccccagagatacaaccgtcttggtcagcccttcctccatcctggaggaaggcagttctgtgaatatgacatgcttgagccagggctttcctgctccgaaaatcctgtggagcaggcagctccctaacggggagctacagcctctttctgagaatgcaactctcaccttaatttctacaaaaatggaagattctggggtttatttatgtgaaggaattaaccaggctggaagaagcagaaaggaagtggaattaattatccaagttactccaaaagacataaaacttacagcttttccttctgagagtgtcaaagaaggagacactgtcatcatctcttgtacatgtggaaatgttccagaaacatggataatcctgaagaaaaaagcggagacaggagacacagtactaaaatctatagatggcgcctataccatccgaaaggcccagttgaaggatgcgggagtatatgaatgtgaatctaaaaacaaagttggctcacaattaagaagtttaacacttgatgttcaaggaagagaaaacaacaaagactatttttctcctgagcttctcgtgctctattttgcatcctccttaataatacctgccattggaatgataatttactttgcaagaaaagccaacatgaaggggtcatatagtcttgtagaagcacagaaatcaaaagtgtagctaatgcttgatatgttcaactggagacactatttatctgtgcaaatccttgatactgctcatcattccttgagaaaaacaatgagctgagaggcagacttccctgaatgtattgaacttggaaagaaatgcccatctatgtcccttgctgtgagcaagaagtcaaagtaaaacttgctgcctgaagaacagtaactgccatcaagatgagagaactggaggagttccttgatctgtatatacaataacataatttgtacatatgtaaaataaaattatgccatagcaagattgcttaaaatagcaacactctatatttagattgttaaaataactagtgttgcttggactattataatttaatgcatgttaggaaaatttcacattaatatttgctgacagctgacctttgtcatctttcttctattttattccctttcacaaaattttattcctatatagtttattgacaataatttcaggttttgtaaagatgccgggttttatatttttatagacaaataataagcaaagggagcactgggttgactttcaggtactaaatacctcaacctatggtataatggttgactgggtttctctgtatagtactggcatggtacggagatgtttcacgaagtttgttcatcagactcctgtgcaactttcccaatgtggcctaaaaatgcaacttctttttattttcttttgtaaatgtttaggtttttttgtatagtaaagtgataatttctggaattagaaaaaaaaaaaaaaaaaaa

SEQ ID NO: 22 - protein, 739 aa, Homo sapiens
  vascular cell adhesion molecule 1 (VCAM1) isoform a presursor
MPGKMVVILGASNILWIMFAASQAFKIETTPESRYLAQIGDSVSLTCSTTGCESPFFSWRTQIDSPLNGKVTNEGTTSTLTMNPVSFGNEHSYLCTATCESRKLEKGIQVEIYSFPKDPEIHLSGPLEAGKPITVKCSVADVYPFDRLEIDLLKGDHLMKSQEFLEDADRKSLETKSLEVTFTPVIEDIGKVLVCRAKLHIDEMDSVPTVRQAVKELQVYISPKNTVISVNPSTKLQEGGSVTMTCSSEGLPAPEIFWSKKLDNGNLQHLSGNATLTLIAMRMEDSGIYVCEGVNLIGKNRKEVELIVQEKPFTVEISPGPRIAAQIGDSVMLTCSVMGCESPSFSWRTQIDSPLSGKVRSEGTNSTLTLSPVSFENEHSYLCTVTCGHKKLEKGIQVELYSFPRDPEIEMSGGLVNGSSVTVSCKVPSVYPLDRLEIELLKGETILENIEFLEDTDMKSLENKSLEMTFIPTIEDTGKALVCQAKLHIDDMEFEPKQRQSTQTLYVNVAPRDTTVLVSPSSILEEGSSVNMTCLSQGFPAPKILWSRQLPNGELQPLSENATLTLISTKMEDSGVYLCEGINQAGRSRKEVELIIQVTPKDIKLTAFPSESVKEGDTVIISCTCGNVPETWIILKKKAETGDTVLKSIDGAYTIRKAQLKDAGVYECESKNKVGSQLRSLTLDVQGRENNKDYFSPELLVLYFASSLIIPAIGMIIYFARKANMKGSYSLVEAQKSKV

SEQ ID NO: 23 - DNA, 4485 bp, Homo sapiens
  nucleotide-binding oligomerization domain containing 2 (NOD2/CARD15) cDNA
gtagacagatccaggctcaccagtcctgtgccactgggcttttggcgttctgcacaaggcctacccgcagatgccatgcctgctcccccagcctaatgggctttgatgggggaagagggtggttcagcctctcacgatgaggaggaaagagcaagtgtcctcctcggacattctccgggttgtgaaatgtgctcgcaggaggcttttcaggcacagaggagccagctggtcgagctgctggtctcagggtccctggaaggcttcgagagtgtcctggactggctgctgtcctgggaggtcctctcctgggaggactacgagggcttccacctcctgggccagcctctctcccacttggccaggcgccttctggacaccgtctggaataagggtacttgggcctgtcagaagctcatcgcggctgcccaagaagcccaggccgacagccagtcccccaagctgcatggctgctgggacccccactcgctccacccagcccgagacctgcagagtcaccggccagccattgtcaggaggctccacagccatgtggagaacatgctggacctggcatgggagcggggtttcgtcagccagtatgaatgtgatgaaatcaggttgccgatcttcacaccgtcccagagggcaagaaggctgcttgatcttgccacggtgaaagcgaatggattggctgccttccttctacaacatgttcaggaattaccagtcccattggccctgcctttggaagctgccacatgcaagaagtatatggccaagctgaggaccacggtgtctgctcagtctcgcttcctcagtacctatgatggagcagagacgctctgcctggaggacatatacacagagaatgtcctggaggtctgggcagatgtgggcatggctggacccccgcagaagagcccagccaccctgggcctggaggagctcttcagcacccctggccacctcaatgacgatgcggacactgtgctggtggtgggtgaggcgggcagtggcaagagcacgctcctgcagcggctgcacttgctgtgggctgcagggcaagacttccaggaatttctctttgtcttcccattcagctgccggcagctgcagtgcatggccaaaccactctctgtgcggactctactctttgagcactgctgttggcctgatgttggtcaagaagacatcttccagttactccttgaccaccctgaccgtgtcctgttaacctttgatggctttgacgagttcaagttcaggttcacggatcgtgaacgccactgctccccgaccgaccccacctctgtccagaccctgctcttcaaccttctgcagggcaacctgctgaagaatgcccgcaaggtggtgaccagccgtccggccgctgtgtcggcgttcctcaggaagtacatccgcaccgagttcaacctcaagggcttctctgaacagggcatcgagctgtacctgaggaagcgccatcatgagcccggggtggcggaccgcctcatccgcctgctccaagagacctcagccctgcacggtttgtgccacctgcctgtcttctcatggatggtgtccaaatgccaccaggaactgttgctgcaggagggggggtccccaaagaccactacagatatgtacctgctgattctgcagcattttctgctgcatgccacccccccagactcagcttcccaaggtctgggacccagtcttcttcggggccgcctccccaccctcctgcacctgggcagactggctctgtggggcctgggcatgtgctgctacgtgttctcagcccagcagctccaggcagcacaggtcagccctgatgacatttctcttggcttcctggtgcgtgccaaaggtgtcgtgccagggagtacggcgcccctggaattccttcacatcactttccagtgcttctttgccgcgttctacctggcactcagtgctgatgtgccaccagctttgctcagacacctcttcaattgtggcaggccaggcaactcaccaatggccaggctcctgcccacgatgtgcatccaggcctcggagggaaaggacagcagcgtggcagctttgctgcagaaggccgagccgcacaaccttcagatcacagcagccttcctggcagggctgttgtcccgggagcactggggcctgctggctgagtgccagacatctgagaaggccctgctccggcgccaggcctgtgcccgctggtgtctggcccgcagcctccgcaagcacttccactccatcccgccagctgcaccgggtgaggccaagagcgtgcatgccatgcccgggttcatctggctcatccggagcctgtacgagatgcaggaggagcggctggctcggaaggctgcacgtggcctgaatgttgggcacctcaagttgacattttgcagtgtgggccccactgagtgtgctgccctggcctttgtgctgcagcacctccggcggcccgtggccctgcagctggactacaactctgtgggtgacattggcgtggagcagctgctgccttgccttggtgtctgcaaggctctgtatttgcgcgataacaatatctcagaccgaggcatctgcaagctcattgaatgtgctcttcactgcgagcaattgcagaagttagctctattcaacaacaaattgactgacggctgtgcacactccatggctaagctccttgcatgcaggcagaacttcttggcattgaggctggggaataactacatcactgccgcgggagcccaagtgctggccgaggggctccgaggcaacacctccttgcagttcctgggattctggggcaacagagtgggtgacgagggggcccaggccctggctgaagccttgggtgatcaccagagcttgaggtggctcagcctggtggggaacaacattggcagtgtgggtgcccaagccttggcactgatgctggcaaagaacgtcatgctagaagaactctgcctggaggagaaccatctccaggatgaaggtgtatgttctctcgcagaaggactgaagaaaaattcaagtttgaaaatcctgaagttgtccaataactgcatcacctacctaggggcagaagccctcctgcaggcccttgaaaggaatgacaccatcctggaagtctggctccgagggaacactttctctctagaggaggttgacaagctcggctgcagggacaccagactcttgctttgaagtctccgggaggatgttcgtctcagtttgtttgtgagcaggctgtgagtttgggccccagaggctgggtgacatgtgttggcagcctcttcaaaatgagccctgtcctgcctaaggctgaacttgttttctgggaacaccataggtcacctttattctggcagaggagggagcatcagtgccctccaggatagacttttcccaagcctacttttgccattgacttcttcccaagattcaatcccaggatgtacaaggacagcccctcctccatagtatgggactggcctctgctgatcctcccaggcttccgtgtgggtcagtggggcccatggatgtgcttgttaactgagtgccttttggtggagaggcccggcctctcacaaaagaccccttaccactgctctgatgaagaggagtacacagaacacataattcaggaagcagctttccccatgtctcgactcatccatccaggccattccccgtctctggttcctcccctcctcctggactcctgcacacgctccttcctctgaggctgaaattcagaatattagtgacctcagctttgatatttcacttacagcacccccaaccctggcacccagggtgggaagggctacaccttagcctgccctcctttccggtgtttaagacatttttggaaggggacacgtgacagccgtttgttccccaagacattctaggtttgcaagaaaaatatgaccacactccagctgggatcacatgtggacttttatttccagtgaaatcagttactcttcagttaagcctttggaaacagctcgactttaaaaagctccaaatgcagctttaaaaaattaatctgggccagaatttcaaacggcctcactaggcttctggttgatgcctgtgaactgaactctgacaacagacttctgaaatagacccacaagaggcagttccatttcatttgtgccagaatgctttaggatgtacagttatggattgaaagtttacaggaaaaaaaattaggccgttccttcaaagcaaatgtcttcctggattattcaaaatgatgtatgttgaagcctttgtaaattgtcagatgctgtgcaaatgttattattttaaacattatgatgtgtgaaaactggttaatatttataggtcactttgttttactgtcttaagtttatactcttatagacaacatggccgtgaactttatgctgtaaataatcagaggggaataaactgttgagtcaaaac

SEQ ID NO: 24 - protein, 1040 aa, Homo sapiens
  nucleotide-binding oligomerization domain containing 2 (NOD2/CARD15)
MGEEGGSASHDEEERASVLLGHSPGCEMCSQEAFQAQRSQLVELLVSGSLEGFESVLDWLLSWEVLSWEDYEGFHLLGQPLSHLARRLLDTVWNKGTWACQKLIAAAQEAQADSQSPKLHGCWDPHSLHPARDLQSHRPAIVRRLHSHVENMLDLAWERGFVSQYECDEIRLPIFTPSQRARRLLDLATVKANGLAAFLLQHVQELPVPLALPLEAATCKKYMAKLRTTVSAQSRFLSTYDGAETLCLEDIYTENVLEVWADVGMAGPPQKSPATLGLEELFSTPGHLNDDADTVLVVGEAGSGKSTLLQRLHLLWAAGQDFQEFLFVFPFSCRQLQCMAKPLSVRTLLFEHCCWPDVGQEDIFQLLLDHPDRVLLTFDGFDEFKFRFTDRERHCSPTDPTSVQTLLFNLLQGNLLKNARKVVTSRPAAVSAFLRKYIRTEFNLKGFSEQGIELYLRKRHHEPGVADRLIRLLQETSALHGLCHLPVFSWMVSKCHQELLLQEGGSPKTTTDMYLLILQHFLLHATPPDSASQGLGPSLLRGRLPTLLHLGRLALWGLGMCCYVFSAQQLQAAQVSPDDISLGFLVRAKGVVPGSTAPLEFLHITFQCFFAAFYLALSADVPPALLRHLFNCGRPGNSPMARLLPTMCIQASEGKDSSVAALLQKAEPHNLQITAAFLAGLLSREHWGLLAECQTSEKALLRRQACARWCLARSLRKHFHSIPPAAPGEAKSVHAMPGFIWLIRSLYEMQEERLARKAARGLNVGHLKLTFCSVGPTECAALAFVLQHLRRPVALQLDYNSVGDIGVEQLLPCLGVCKALYLRDNNISDRGICKLIECALHCEQLQKLALFNNKLTDGCAHSMAKLLACRQNFLALRLGNNYITAAGAQVLAEGLRGNTSLQFLGFWGNRVGDEGAQALAEALGDHQSLRWLSLVGNNIGSVGAQALALMLAKNVMLEELCLEENHLQDEGVCSLAEGLKKNSSLKILKLSNNCITYLGAEALLQALERNDTILEVWLRGNTFSLEEVDKLGCRDTRLLL

SEQ ID NO: 25 - DNA, 3618 bp, Homo sapiens
  GLI family zinc finger 1 (GLI1), transcript variant 1 cDNA
cccagactccagccctggaccgcgcatcccgagcccagcgcccagacagagtgtccccacaccctcctctgagacgccatgttcaactcgatgaccccaccaccaatcagtagctatggcgagccctgctgtctccggcccctccccagtcagggggcccccagtgtggggacagaaggactgtctggcccgcccttctgccaccaagctaacctcatgtccggcccccacagttatgggccagccagagagaccaacagctgcaccgagggcccactcttttcttctccccggagtgcagtcaagttgaccaagaagcgggcactgtccatctcacctctgtcggatgccagcctggacctgcagacggttatccgcacctcacccagctccctcgtagctttcatcaactcgcgatgcacatctccaggaggctcctacggtcatctctccattggcaccatgagcccatctctgggattcccagcccagatgaatcaccaaaaagggccctcgccttcctttggggtccagccttgtggtccccatgactctgcccggggtgggatgatcccacatcctcagtcccggggacccttcccaacttgccagctgaagtctgagctggacatgctggttggcaagtgccgggaggaacccttggaaggtgatatgtccagccccaactccacaggcatacaggatcccctgttggggatgctggatgggcgggaggacctcgagagagaggagaagcgtgagcctgaatctgtgtatgaaactgactgccgttgggatggctgcagccaggaatttgactcccaagagcagctggtgcaccacatcaacagcgagcacatccacggggagcggaaggagttcgtgtgccactgggggggctgctccagggagctgaggcccttcaaagcccagtacatgctggtggttcacatgcgcagacacactggcgagaagccacacaagtgcacgtttgaagggtgccggaagtcatactcacgcctcgaaaacctgaagacgcacctgcggtcacacacgggtgagaagccatacatgtgtgagcacgagggctgcagtaaagccttcagcaatgccagtgaccgagccaagcaccagaatcggacccattccaatgagaagccgtatgtatgtaagctccctggctgcaccaaacgctatacagatcctagctcgctgcgaaaacatgtcaagacagtgcatggtcctgacgcccatgtgaccaaacggcaccgtggggatggccccctgcctcgggcaccatccatttctacagtggagcccaagagggagcgggaaggaggtcccatcagggaggaaagcagactgactgtgccagagggtgccatgaagccacagccaagccctggggcccagtcatcctgcagcagtgaccactccccggcagggagtgcagccaatacagacagtggtgtggaaatgactggcaatgcagggggcagcactgaagacctctccagcttggacgagggaccttgcattgctggcactggtctgtccactcttcgccgccttgagaacctcaggctggaccagctacatcaactccggccaatagggacccggggtctcaaactgcccagcttgtcccacaccggtaccactgtgtcccgccgcgtgggccccccagtctctcttgaacgccgcagcagcagctccagcagcatcagctctgcctatactgtcagccgccgctcctccctggcctctcctttcccccctggctccccaccagagaatggagcatcctccctgcctggccttatgcctgcccagcactacctgcttcgggcaagatatgcttcagccagagggggtggtacttcgcccactgcagcatccagcctggatcggataggtggtcttcccatgcctccttggagaagccgagccgagtatccaggatacaaccccaatgcaggggtcacccggagggccagtgacccagcccaggctgctgaccgtcctgctccagctagagtccagaggttcaagagcctgggctgtgtccataccccacccactgtggcagggggaggacagaactttgatccttacctcccaacctctgtctactcaccacagccccccagcatcactgagaatgctgccatggatgctagagggctacaggaagagccagaagttgggacctccatggtgggcagtggtctgaacccctatatggacttcccacctactgatactctgggatatgggggacctgaaggggcagcagctgagccttatggagcgaggggtccaggctctctgcctcttgggcctggtccacccaccaactatggccccaacccctgtccccagcaggcctcatatcctgaccccacccaagaaacatggggtgagttcccttcccactctgggctgtacccaggccccaaggctctaggtggaacctacagccagtgtcctcgacttgaacattatggacaagtgcaagtcaagccagaacaggggtgcccagtggggtctgactccacaggactggcaccctgcctcaatgcccaccccagtgaggggcccccacatccacagcctctcttttcccattacccccagccctctcctccccaatatctccagtcaggcccctatacccagccaccccctgattatcttccttcagaacccaggccttgcctggactttgattcccccacccattccacagggcagctcaaggctcagcttgtgtgtaattatgttcaatctcaacaggagctactgtgggagggtgggggcagggaagatgcccccgcccaggaaccttcctaccagagtcccaagtttctggggggttcccaggttagcccaagccgtgctaaagctccagtgaacacatatggacctggctttggacccaacttgcccaatcacaagtcaggttcctatcccaccccttcaccatgccatgaaaattttgtagtgggggcaaatagggcttcacatagggcagcagcaccacctcgacttctgcccccattgcccacttgctatgggcctctcaaagtgggaggcacaaaccccagctgtggtcatcctgaggtgggcaggctaggagggggtcctgccttgtaccctcctcccgaaggacaggtatgtaaccccctggactctcttgatcttgacaacactcagctggactttgtggctattctggatgagccccaggggctgagtcctcctccttcccatgatcagcggggcagctctggacataccccacctccctctgggccccccaacatggctgtgggcaacatgagtgtcttactgagatccctacctggggaaacagaattcctcaactctagtgcctaaagagtagggaatctcatccatcacagatcgcatttcctaaggggtttctatccttccagaaaaattgggggagctgcagtcccatgcacaagatgccccagggatgggaggtatgggctgggggctatgtatagtctgtatacgttttgaggagaaatttgataatgacactgtttcctgataataaaggaactgcatcagaaaaaaaaaaaaaaaaaa

SEQ ID NO: 26 - protein, 1106 aa, Homo sapiens
  GLI family zinc finger 1 (GLI1) isoform 1
MFNSMTPPPISSYGEPCCLRPLPSQGAPSVGTEGLSGPPFCHQANLMSGPHSYGPARETNSCTEGPLFSSPRSAVKLTKKRALSISPLSDASLDLQTVIRTSPSSLVAFINSRCTSPGGSYGHLSIGTMSPSLGFPAQMNHQKGPSPSFGVQPCGPHDSARGGMIPHPQSRGPFPTCQLKSELDMLVGKCREEPLEGDMSSPNSTGIQDPLLGMLDGREDLEREEKREPESVYETDCRWDGCSQEFDSQEQLVHHINSEHIHGERKEFVCHWGGCSRELRPFKAQYMLVVHMRRHTGEKPHKCTFEGCRKSYSRLENLKTHLRSHTGEKPYMCEHEGCSKAFSNASDRAKHQNRTHSNEKPYVCKLPGCTKRYTDPSSLRKHVKTVHGPDAHVTKRHRGDGPLPRAPSISTVEPKREREGGPIREESRLTVPEGAMKPQPSPGAQSSCSSDHSPAGSAANTDSGVEMTGNAGGSTEDLSSLDEGPCIAGTGLSTLRRLENLRLDQLHQLRPIGTRGLKLPSLSHTGTTVSRRVGPPVSLERRSSSSSSISSAYTVSRRSSLASPFPPGSPPENGASSLPGLMPAQHYLLRARYASARGGGTSPTAASSLDRIGGLPMPPWRSRAEYPGYNPNAGVTRRASDPAQAADRPAPARVQRFKSLGCVHTPPTVAGGGQNFDPYLPTSVYSPQPPSITENAAMDARGLQEEPEVGTSMVGSGLNPYMDFPPTDTLGYGGPEGAAAEPYGARGPGSLPLGPGPPTNYGPNPCPQQASYPDPTQETWGEFPSHSGLYPGPKALGGTYSQCPRLEHYGQVQVKPEQGCPVGSDSTGLAPCLNAHPSEGPPHPQPLFSHYPQPSPPQYLQSGPYTQPPPDYLPSEPRPCLDFDSPTHSTGQLKAQLVCNYVQSQQELLWEGGGREDAPAQEPSYQSPKFLGGSQVSPSRAKAPVNTYGPGFGPNLPNHKSGSYPTPSPCHENFVVGANRASHRAAAPPRLLPPLPTCYGPLKVGGTNPSCGHPEVGRLGGGPALYPPPEGQVCNPLDSLDLDNTQLDFVAILDEPQGLSPPPSHDQRGSSGHTPPPSGPPNMAVGNMSVLLRSLPGETEFLNSSA

SEQ ID NO: 27 - DNA, 4872 bp, Homo sapiens
  ATP-binding cassette, sub-family B (MDR/TAP), member 1 (ABCB1), multi-drug resistance protein 1 (MDR1), permeability glycoprotein (P-glycoprotein), PGY1 cDNA
tattcagatattctccagattcctaaagattagagatcatttctcattctcctaggagtactcacttcaggaagcaaccagataaaagagaggtgcaacggaagccagaacattcctcctggaaattcaacctgtttcgcagtttctcgaggaatcagcattcagtcaatccgggccgggagcagtcatctgtggtgaggctgattggctgggcaggaacagcgccggggcgtgggctgagcacagccgcttcgctctctttgccacaggaagcctgagctcattcgagtagcggctcttccaagctcaaagaagcagaggccgctgttcgtttcctttaggtctttccactaaagtcggagtatcttcttccaaaatttcacgtcttggtggccgttccaaggagcgcgaggtcggaatggatcttgaaggggaccgcaatggaggagcaaagaagaagaacttttttaaactgaacaataaaagtgaaaaagataagaaggaaaagaaaccaactgtcagtgtattttcaatgtttcgctattcaaattggcttgacaagttgtatatggtggtgggaactttggctgccatcatccatggggctggacttcctctcatgatgctggtgtttggagaaatgacagatatctttgcaaatgcaggaaatttagaagatctgatgtcaaacatcactaatagaagtgatatcaatgatacagggttcttcatgaatctggaggaagacatgaccaggtatgcctattattacagtggaattggtgctggggtgctggttgctgcttacattcaggtttcattttggtgcctggcagctggaagacaaatacacaaaattagaaaacagttttttcatgctataatgcgacaggagataggctggtttgatgtgcacgatgttggggagcttaacacccgacttacagatgatgtctccaagattaatgaaggaattggtgacaaaattggaatgttctttcagtcaatggcaacatttttcactgggtttatagtaggatttacacgtggttggaagctaacccttgtgattttggccatcagtcctgttcttggactgtcagctgctgtctgggcaaagatactatcttcatttactgataaagaactcttagcgtatgcaaaagctggagcagtagctgaagaggtcttggcagcaattagaactgtgattgcatttggaggacaaaagaaagaacttgaaaggtacaacaaaaatttagaagaagctaaaagaattgggataaagaaagctattacagccaatatttctataggtgctgctttcctgctgatctatgcatcttatgctctggccttctggtatgggaccaccttggtcctctcaggggaatattctattggacaagtactcactgtattcttttctgtattaattggggcttttagtgttggacaggcatctccaagcattgaagcatttgcaaatgcaagaggagcagcttatgaaatcttcaagataattgataataagccaagtattgacagctattcgaagagtgggcacaaaccagataatattaagggaaatttggaattcagaaatgttcacttcagttacccatctcgaaaagaagttaagatcttgaagggtctgaacctgaaggtgcagagtgggcagacggtggccctggttggaaacagtggctgtgggaagagcacaacagtccagctgatgcagaggctctatgaccccacagaggggatggtcagtgttgatggacaggatattaggaccataaatgtaaggtttctacgggaaatcattggtgtggtgagtcaggaacctgtattgtttgccaccacgatagctgaaaacattcgctatggccgtgaaaatgtcaccatggatgagattgagaaagctgtcaaggaagccaatgcctatgactttatcatgaaactgcctcataaatttgacaccctggttggagagagaggggcccagttgagtggtgggcagaagcagaggatcgccattgcacgtgccctggttcgcaaccccaagatcctcctgctggatgaggccacgtcagccttggacacagaaagcgaagcagtggttcaggtggctctggataaggccagaaaaggtcggaccaccattgtgatagctcatcgtttgtctacagttcgtaatgctgacgtcatcgctggtttcgatgatggagtcattgtggagaaaggaaatcatgatgaactcatgaaagagaaaggcatttacttcaaacttgtcacaatgcagacagcaggaaatgaagttgaattagaaaatgcagctgatgaatccaaaagtgaaattgatgccttggaaatgtcttcaaatgattcaagatccagtctaataagaaaaagatcaactcgtaggagtgtccgtggatcacaagcccaagacagaaagcttagtaccaaagaggctctggatgaaagtatacctccagtttccttttggaggattatgaagctaaatttaactgaatggccttattttgttgttggtgtattttgtgccattataaatggaggcctgcaaccagcatttgcaataatattttcaaagattataggggtttttacaagaattgatgatcctgaaacaaaacgacagaatagtaacttgttttcactattgtttctagcccttggaattatttcttttattacatttttccttcagggtttcacatttggcaaagctggagagatcctcaccaagcggctccgatacatggttttccgatccatgctcagacaggatgtgagttggtttgatgaccctaaaaacaccactggagcattgactaccaggctcgccaatgatgctgctcaagttaaaggggctataggttccaggcttgctgtaattacccagaatatagcaaatcttgggacaggaataattatatccttcatctatggttggcaactaacactgttactcttagcaattgtacccatcattgcaatagcaggagttgttgaaatgaaaatgttgtctggacaagcactgaaagataagaaagaactagaaggttctgggaagatcgctactgaagcaatagaaaacttccgaaccgttgtttctttgactcaggagcagaagtttgaacatatgtatgctcagagtttgcaggtaccatacagaaactctttgaggaaagcacacatctttggaattacattttccttcacccaggcaatgatgtatttttcctatgctggatgtttccggtttggagcctacttggtggcacataaactcatgagctttgaggatgttctgttagtattttcagctgttgtctttggtgccatggccgtggggcaagtcagttcatttgctcctgactatgccaaagccaaaatatcagcagcccacatcatcatgatcattgaaaaaacccctttgattgacagctacagcacggaaggcctaatgccgaacacattggaaggaaatgtcacatttggtgaagttgtattcaactatcccacccgaccggacatcccagtgcttcagggactgagcctggaggtgaagaagggccagacgctggctctggtgggcagcagtggctgtgggaagagcacagtggtccagctcctggagcggttctacgaccccttggcagggaaagtgctgcttgatggcaaagaaataaagcgactgaatgttcagtggctccgagcacacctgggcatcgtgtcccaggagcccatcctgtttgactgcagcattgctgagaacattgcctatggagacaacagccgggtggtgtcacaggaagagattgtgagggcagcaaaggaggccaacatacatgccttcatcgagtcactgcctaataaatatagcactaaagtaggagacaaaggaactcagctctctggtggccagaaacaacgcattgccatagctcgtgcccttgttagacagcctcatattttgcttttggatgaagccacgtcagctctggatacagaaagtgaaaaggttgtccaagaagccctggacaaagccagagaaggccgcacctgcattgtgattgctcaccgcctgtccaccatccagaatgcagacttaatagtggtgtttcagaatggcagagtcaaggagcatggcacgcatcagcagctgctggcacagaaaggcatctatttttcaatggtcagtgtccaggctggaacaaagcgccagtgaactctgactgtatgagatgttaaatactttttaatatttgtttagatatgacatttattcaaagttaaaagcaaacacttacagaattatgaagaggtatctgtttaacatttcctcagtcaagttcagagtcttcagagacttcgtaattaaaggaacagagtgagagacatcatcaagtggagagaaatcatagtttaaactgcattataaattttataacagaattaaagtagattttaaaagataaaatgtgtaattttgtttatattttcccatttggactgtaactgactgccttgctaaaagattatagaagtagcaaaaagtattgaaatgtttgcataaagtgtctataataaaactaaactttcatgtgactggagtcatcttgtccaaactgcctgtgaatatatcttctctcaattggaatattgtagataacttctgctttaaaaaagttttctttaaatatacctactcatttttgtgggaatggttaagcagtttaaataattcctgttgtatatgtctattcacattgggtcttacagaaccatctggcttcattcttcttggacttgatcctgctgattcttgcatttccacat

SEQ ID NO: 28 - protein, 1280 aa, Homo sapiens
  ATP-binding cassette, sub-family B (MDR/TAP), member 1 (ABCB1), multi-drug resistance protein 1 (MDR1), permeability glycoprotein (P-glycoprotein), PGY1
MDLEGDRNGGAKKKNFFKLNNKSEKDKKEKKPTVSVFSMFRYSNWLDKLYMVVGTLAAIIHGAGLPLMMLVFGEMTDIFANAGNLEDLMSNITNRSDINDTGFFMNLEEDMTRYAYYYSGIGAGVLVAAYIQVSFWCLAAGRQIHKIRKQFFHAIMRQEIGWFDVHDVGELNTRLTDDVSKINEGIGDKIGMFFQSMATFFTGFIVGFTRGWKLTLVILAISPVLGLSAAVWAKILSSFTDKELLAYAKAGAVAEEVLAAIRTVIAFGGQKKELERYNKNLEEAKRIGIKKAITANISIGAAFLLIYASYALAFWYGTTLVLSGEYSIGQVLTVFFSVLIGAFSVGQASPSIEAFANARGAAYEIFKIIDNKPSIDSYSKSGHKPDNIKGNLEFRNVHFSYPSRKEVKILKGLNLKVQSGQTVALVGNSGCGKSTTVQLMQRLYDPTEGMVSVDGQDIRTINVRFLREIIGVVSQEPVLFATTIAENIRYGRENVTMDEIEKAVKEANAYDFIMKLPHKFDTLVGERGAQLSGGQKQRIAIARALVRNPKILLLDEATSALDTESEAVVQVALDKARKGRTTIVIAHRLSTVRNADVIAGFDDGVIVEKGNHDELMKEKGIYFKLVTMQTAGNEVELENAADESKSEIDALEMSSNDSRSSLIRKRSTRRSVRGSQAQDRKLSTKEALDESIPPVSFWRIMKLNLTEWPYFVVGVFCAIINGGLQPAFAIIFSKIIGVFTRIDDPETKRQNSNLFSLLFLALGIISFITFFLQGFTFGKAGEILTKRLRYMVFRSMLRQDVSWFDDPKNTTGALTTRLANDAAQVKGAIGSRLAVITQNIANLGTGIIISFIYGWQLTLLLLAIVPIIAIAGVVEMKMLSGQALKDKKELEGSGKIATEAIENFRTVVSLTQEQKFEHMYAQSLQVPYRNSLRKAHIFGITFSFTQAMMYFSYAGCFRFGAYLVAHKLMSFEDVLLVFSAVVFGAMAVGQVSSFAPDYAKAKISAAHIIMIIEKTPLIDSYSTEGLMPNTLEGNVTFGEVVFNYPTRPDIPVLQGLSLEVKKGQTLALVGSSGCGKSTVVQLLERFYDPLAGKVLLDGKEIKRLNVQWLRAHLGIVSQEPILFDCSIAENIAYGDNSRVVSQEEIVRAAKEANIHAFIESLPNKYSTKVGDKGTQLSGGQKQRIAIARALVRQPHILLLDEATSALDTESEKVVQEALDKAREGRTCIVIAHRLSTIQNADLIVVFQNGRVKEHGTHQQLLAQKGIYFSMVSVQAGTKRQ

SEQ ID NO: 29 - DNA, 3354 bp, Homo sapiens
  ATG16 autophagy related 16-like 1 (S. cerevisiae) (ATG16L1), transcript variant 2 cDNA
actagcgagcgccctgcgtaggcaccggctcctgagcccgtgcttcgggtgagggggcgggtcttccggccctctcgaaaatcatttccggcatgagccggaagaccgtcccggatggcctcggggactgccagtgtgtggaggtgagctccgggattgccggcattcccgcttctgctggttgcttcatgctgcaggctgcggccgtcagccctcgctcgcattggtggcgctgaggtgccggggcagcaagtgacatgtcgtcgggcctccgcgccgctgacttcccccgctggaagcgccacatctcggagcaactgaggcgccgggaccggctgcagagacaggcgttcgaggagatcatcctgcagtataacaaattgctggaaaagtcagatcttcattcagtgttggcccagaaactacaggctgaaaagcatgacgtaccaaacaggcacgagataagtcccggacatgatggcacatggaatgacaatcagctacaagaaatggcccaactgaggattaagcaccaagaggaactgactgaattacacaagaaacgtggggagttagctcaactggtgattgacctgaataaccaaatgcagcggaaggacagggagatgcagatgaatgaagcaaaaattgcagaatgtttgcagactatctctgacctggagacggagtgcctagacctgcgcactaagctttgtgaccttgaaagagccaaccagaccctgaaggatgaatatgatgccctgcagatcacttttactgccttggagggaaaactgaggaaaactacggaagagaaccaggagctggtcaccagatggatggctgagaaagcccaggaagccaatcggcttaatgcagagaatgaaaaagactccaggaggcggcaagcccggctgcagaaagagcttgcagaagcagcaaaggaacctctaccagtcgaacaggatgatgacattgaggtcattgtggatgaaacttctgatcacacagaagagacctctcctgtgcgagccatcagcagagcagccacgagacgctctgtctcttccttcccagtcccccaggacaatgtggatactcatcctggttctggtaaagaagtgagggtaccagctactgccttgtgtgtcttcgatgcacatgatggggaagtcaacgctgtgcagttcagtccaggttcccggttactggccactggaggcatggaccgcagggttaagctttgggaagtatttggagaaaaatgtgagttcaagggttccctatctggcagtaatgcaggaattacaagcattgaatttgatagtgctggatcttacctcttagcagcttcaaatgattttgcaagccgaatctggactgtggatgattatcgattacggcacacactcacgggacacagtgggaaagtgctgtctgctaagttcctgctggacaatgcgcggattgtctcaggaagtcacgaccggactctcaaactctgggatctacgcagcaaagtctgcataaagacagtgtttgcaggatccagttgcaatgatattgtctgcacagagcaatgtgtaatgagtggacattttgacaagaaaattcgtttctgggacattcgatcagagagcatagttcgagagatggagctgttgggaaagattactgccctggacttaaacccagaaaggactgagctcctgagctgctcccgtgatgacttgctaaaagttattgatctccgaacaaatgctatcaagcagacattcagtgcacctgggttcaagtgcggctctgactggaccagagttgtcttcagccctgatggcagttacgtggcggcaggctctgctgagggctctctgtatatctggagtgtgctcacagggaaagtggaaaaggttctttcaaagcagcacagctcatccatcaatgcggtggcgtggtcgccctctggctcgcacgttgtcagtgtggacaaaggatgcaaagctgtgctgtgggcacagtactgacggggctctcagggctgggaggaccccagtgccctcctcagaagaagcacatgggctcctgcagccctgtcctggcaggtgatgtgctgggtatagcatggacctcccagagaagctcaagctatgtggcactgtagctttgccgtgaatgggatttctgaagatttgactgaggtctctcttggcctggaagaataacactgaaaaaacctgacgctgcggtcacttagcagaggctcaggttcttgccttgggaaacactactagctctgaccttccatacctcacttgggggagcacagggccccgctgggcctcctcaccaacggcagtgccaaaatcagcccccacatcaaggtggtgttctctgtgctttctctcgtccttccaaagtcggttctggcctaacgcatgtcccaacaccttgggttcatttgcccggtgaactcactttaagcattggattaacggaaactcccgaactacagacccctccctggtgggttgcatgaatgtgtctcattactgctgaaatgtcctcacatctctttcactgttcttcagagctttctggctctctttcccccacaaaattcgacatatttaaaaatctccgtgtggctttaaaaaatggttttttgtttttttgtttttttgaggtgggagaggatgtgtgaaaatcttttccagggaaatgggttcgctgcagaggtaaggatgtgttcctgtatcgatctgcagacacccagaaggtgggtgcacactgcatgcttgggggtgccaagggattcgagacctccaacatacttgtctgaaggtggtgattctggccatggcccctctgccaagcctgtgtgcgatgcccttggtgctttagtgcaagaagcctaggctcagaagcacagcagcgccatctttccgtttcaggggttgtgatgaaggccaaggaaaaacatttatctttactattttacctacgtataaagttttagttcattgggtgtgcgaaacaccctttttatcacttttaaatttgcactttattttttttcttccatgcttgttctctggacatttggggatgtgagtgttagagctggtgagagaggagtcaggtggccttcccaccgatggtcctggcctccacctgccctctcttccctgcctgatcaccgctttccaatttgcccttcagagaacttaagtcaaggagagttgaaattcacaggccagggcacatcttttatttatttcattatgttggccaacagaacttgattgtaaataataataaagaaatctgttatatacttttcaaactccaaaaaaa

SEQ ID NO: 30 - protein, 588 aa, Homo sapiens
  ATG16 autophagy related 16-like 1 (S. cerevisiae) (ATG16L1) isoform 2
MSSGLRAADFPRWKRHISEQLRRRDRLQRQAFEEIILQYNKLLEKSDLHSVLAQKLQAEKHDVPNRHEISPGHDGTWNDNQLQEMAQLRIKHQEELTELHKKRGELAQLVIDLNNQMQRKDREMQMNEAKIAECLQTISDLETECLDLRTKLCDLERANQTLKDEYDALQITFTALEGKLRKTTEENQELVTRWMAEKAQEANRLNAENEKDSRRRQARLQKELAEAAKEPLPVEQDDDIEVIVDETSDHTEETSPVRAISRAATRRSVSSFPVPQDNVDTHPGSGKEVRVPATALCVFDAHDGEVNAVQFSPGSRLLATGGMDRRVKLWEVFGEKCEFKGSLSGSNAGITSIEFDSAGSYLLAASNDFASRIWTVDDYRLRHTLTGHSGKVLSAKFLLDNARIVSGSHDRTLKLWDLRSKVCIKTVFAGSSCNDIVCTEQCVMSGHFDKKIRFWDIRSESIVREMELLGKITALDLNPERTELLSCSRDDLLKVIDLRTNAIKQTFSAPGFKCGSDWTRVVFSPDGSYVAAGSAEGSLYIWSVLTGKVEKVLSKQHSSSINAVAWSPSGSHVVSVDKGCKAVLWAQY

SEQ ID NO: 31 - DNA, 3411 bp, Homo sapiens
  ATG16 autophagy related 16-like 1 (S. cerevisiae) (ATG16L1), transcript variant 1 cDNA
actagcgagcgccctgcgtaggcaccggctcctgagcccgtgcttcgggtgagggggcgggtcttccggccctctcgaaaatcatttccggcatgagccggaagaccgtcccggatggcctcggggactgccagtgtgtggaggtgagctccgggattgccggcattcccgcttctgctggttgcttcatgctgcaggctgcggccgtcagccctcgctcgcattggtggcgctgaggtgccggggcagcaagtgacatgtcgtcgggcctccgcgccgctgacttcccccgctggaagcgccacatctcggagcaactgaggcgccgggaccggctgcagagacaggcgttcgaggagatcatcctgcagtataacaaattgctggaaaagtcagatcttcattcagtgttggcccagaaactacaggctgaaaagcatgacgtaccaaacaggcacgagataagtcccggacatgatggcacatggaatgacaatcagctacaagaaatggcccaactgaggattaagcaccaagaggaactgactgaattacacaagaaacgtggggagttagctcaactggtgattgacctgaataaccaaatgcagcggaaggacagggagatgcagatgaatgaagcaaaaattgcagaatgtttgcagactatctctgacctggagacggagtgcctagacctgcgcactaagctttgtgaccttgaaagagccaaccagaccctgaaggatgaatatgatgccctgcagatcacttttactgccttggagggaaaactgaggaaaactacggaagagaaccaggagctggtcaccagatggatggctgagaaagcccaggaagccaatcggcttaatgcagagaatgaaaaagactccaggaggcggcaagcccggctgcagaaagagcttgcagaagcagcaaaggaacctctaccagtcgaacaggatgatgacattgaggtcattgtggatgaaacttctgatcacacagaagagacctctcctgtgcgagccatcagcagagcagccactaagcgactctcgcagcctgctggaggccttctggattctatcactaatatctttgggagacgctctgtctcttccttcccagtcccccaggacaatgtggatactcatcctggttctggtaaagaagtgagggtaccagctactgccttgtgtgtcttcgatgcacatgatggggaagtcaacgctgtgcagttcagtccaggttcccggttactggccactggaggcatggaccgcagggttaagctttgggaagtatttggagaaaaatgtgagttcaagggttccctatctggcagtaatgcaggaattacaagcattgaatttgatagtgctggatcttacctcttagcagcttcaaatgattttgcaagccgaatctggactgtggatgattatcgattacggcacacactcacgggacacagtgggaaagtgctgtctgctaagttcctgctggacaatgcgcggattgtctcaggaagtcacgaccggactctcaaactctgggatctacgcagcaaagtctgcataaagacagtgtttgcaggatccagttgcaatgatattgtctgcacagagcaatgtgtaatgagtggacattttgacaagaaaattcgtttctgggacattcgatcagagagcatagttcgagagatggagctgttgggaaagattactgccctggacttaaacccagaaaggactgagctcctgagctgctcccgtgatgacttgctaaaagttattgatctccgaacaaatgctatcaagcagacattcagtgcacctgggttcaagtgcggctctgactggaccagagttgtcttcagccctgatggcagttacgtggcggcaggctctgctgagggctctctgtatatctggagtgtgctcacagggaaagtggaaaaggttctttcaaagcagcacagctcatccatcaatgcggtggcgtggtcgccctctggctcgcacgttgtcagtgtggacaaaggatgcaaagctgtgctgtgggcacagtactgacggggctctcagggctgggaggaccccagtgccctcctcagaagaagcacatgggctcctgcagccctgtcctggcaggtgatgtgctgggtatagcatggacctcccagagaagctcaagctatgtggcactgtagctttgccgtgaatgggatttctgaagatttgactgaggtctctcttggcctggaagaataacactgaaaaaacctgacgctgcggtcacttagcagaggctcaggttcttgccttgggaaacactactagctctgaccttccatacctcacttgggggagcacagggccccgctgggcctcctcaccaacggcagtgccaaaatcagcccccacatcaaggtggtgttctctgtgctttctctcgtccttccaaagtcggttctggcctaacgcatgtcccaacaccttgggttcatttgcccggtgaactcactttaagcattggattaacggaaactcccgaactacagacccctccctggtgggttgcatgaatgtgtctcattactgctgaaatgtcctcacatctctttcactgttcttcagagctttctggctctctttcccccacaaaattcgacatatttaaaaatctccgtgtggctttaaaaaatggttttttgtttttttgtttttttgaggtgggagaggatgtgtgaaaatcttttccagggaaatgggttcgctgcagaggtaaggatgtgttcctgtatcgatctgcagacacccagaaggtgggtgcacactgcatgcttgggggtgccaagggattcgagacctccaacatacttgtctgaaggtggtgattctggccatggcccctctgccaagcctgtgtgcgatgcccttggtgctttagtgcaagaagcctaggctcagaagcacagcagcgccatctttccgtttcaggggttgtgatgaaggccaaggaaaaacatttatctttactattttacctacgtataaagttttagttcattgggtgtgcgaaacaccctttttatcacttttaaatttgcactttattttttttcttccatgcttgttctctggacatttggggatgtgagtgttagagctggtgagagaggagtcaggtggccttcccaccgatggtcctggcctccacctgccctctcttccctgcctgatcaccgctttccaatttgcccttcagagaacttaagtcaaggagagttgaaattcacaggccagggcacatcttttatttatttcattatgttggccaacagaacttgattgtaaataataataaagaaatctgttatatacttttcaaactccaaaaaaa

SEQ ID NO: 32 - protein, 607 aa, Homo sapiens
  ATG16 autophagy related 16-like 1 (S. cerevisiae) (ATG16L1) isoform 1
MSSGLRAADFPRWKRHISEQLRRRDRLQRQAFEEIILQYNKLLEKSDLHSVLAQKLQAEKHDVPNRHEISPGHDGTWNDNQLQEMAQLRIKHQEELTELHKKRGELAQLVIDLNNQMQRKDREMQMNEAKIAECLQTISDLETECLDLRTKLCDLERANQTLKDEYDALQITFTALEGKLRKTTEENQELVTRWMAEKAQEANRLNAENEKDSRRRQARLQKELAEAAKEPLPVEQDDDIEVIVDETSDHTEETSPVRAISRAATKRLSQPAGGLLDSITNIFGRRSVSSFPVPQDNVDTHPGSGKEVRVPATALCVFDAHDGEVNAVQFSPGSRLLATGGMDRRVKLWEVFGEKCEFKGSLSGSNAGITSIEFDSAGSYLLAASNDFASRIWTVDDYRLRHTLTGHSGKVLSAKFLLDNARIVSGSHDRTLKLWDLRSKVCIKTVFAGSSCNDIVCTEQCVMSGHFDKKIRFWDIRSESIVREMELLGKITALDLNPERTELLSCSRDDLLKVIDLRTNAIKQTFSAPGFKCGSDWTRVVFSPDGSYVAAGSAEGSLYIWSVLTGKVEKVLSKQHSSSINAVAWSPSGSHVVSVDKGCKAVLWAQY

SEQ ID NO: 33 - DNA, 3414 bp, Homo sapiens
  GLI family zinc finger 1 (GLI1), transcript variant 2 cDNA
accgcacaccccccagcccagactccagccctggaccgcgcatcccgagcccagcgcccagacagaggcccactcttttcttctccccggagtgcagtcaagttgaccaagaagcgggcactgtccatctcacctctgtcggatgccagcctggacctgcagacggttatccgcacctcacccagctccctcgtagctttcatcaactcgcgatgcacatctccaggaggctcctacggtcatctctccattggcaccatgagcccatctctgggattcccagcccagatgaatcaccaaaaagggccctcgccttcctttggggtccagccttgtggtccccatgactctgcccggggtgggatgatcccacatcctcagtcccggggacccttcccaacttgccagctgaagtctgagctggacatgctggttggcaagtgccgggaggaacccttggaaggtgatatgtccagccccaactccacaggcatacaggatcccctgttggggatgctggatgggcgggaggacctcgagagagaggagaagcgtgagcctgaatctgtgtatgaaactgactgccgttgggatggctgcagccaggaatttgactcccaagagcagctggtgcaccacatcaacagcgagcacatccacggggagcggaaggagttcgtgtgccactgggggggctgctccagggagctgaggcccttcaaagcccagtacatgctggtggttcacatgcgcagacacactggcgagaagccacacaagtgcacgtttgaagggtgccggaagtcatactcacgcctcgaaaacctgaagacgcacctgcggtcacacacgggtgagaagccatacatgtgtgagcacgagggctgcagtaaagccttcagcaatgccagtgaccgagccaagcaccagaatcggacccattccaatgagaagccgtatgtatgtaagctccctggctgcaccaaacgctatacagatcctagctcgctgcgaaaacatgtcaagacagtgcatggtcctgacgcccatgtgaccaaacggcaccgtggggatggccccctgcctcgggcaccatccatttctacagtggagcccaagagggagcgggaaggaggtcccatcagggaggaaagcagactgactgtgccagagggtgccatgaagccacagccaagccctggggcccagtcatcctgcagcagtgaccactccccggcagggagtgcagccaatacagacagtggtgtggaaatgactggcaatgcagggggcagcactgaagacctctccagcttggacgagggaccttgcattgctggcactggtctgtccactcttcgccgccttgagaacctcaggctggaccagctacatcaactccggccaatagggacccggggtctcaaactgcccagcttgtcccacaccggtaccactgtgtcccgccgcgtgggccccccagtctctcttgaacgccgcagcagcagctccagcagcatcagctctgcctatactgtcagccgccgctcctccctggcctctcctttcccccctggctccccaccagagaatggagcatcctccctgcctggccttatgcctgcccagcactacctgcttcgggcaagatatgcttcagccagagggggtggtacttcgcccactgcagcatccagcctggatcggataggtggtcttcccatgcctccttggagaagccgagccgagtatccaggatacaaccccaatgcaggggtcacccggagggccagtgacccagcccaggctgctgaccgtcctgctccagctagagtccagaggttcaagagcctgggctgtgtccataccccacccactgtggcagggggaggacagaactttgatccttacctcccaacctctgtctactcaccacagccccccagcatcactgagaatgctgccatggatgctagagggctacaggaagagccagaagttgggacctccatggtgggcagtggtctgaacccctatatggacttcccacctactgatactctgggatatgggggacctgaaggggcagcagctgagccttatggagcgaggggtccaggctctctgcctcttgggcctggtccacccaccaactatggccccaacccctgtccccagcaggcctcatatcctgaccccacccaagaaacatggggtgagttcccttcccactctgggctgtacccaggccccaaggctctaggtggaacctacagccagtgtcctcgacttgaacattatggacaagtgcaagtcaagccagaacaggggtgcccagtggggtctgactccacaggactggcaccctgcctcaatgcccaccccagtgaggggcccccacatccacagcctctcttttcccattacccccagccctctcctccccaatatctccagtcaggcccctatacccagccaccccctgattatcttccttcagaacccaggccttgcctggactttgattcccccacccattccacagggcagctcaaggctcagcttgtgtgtaattatgttcaatctcaacaggagctactgtgggagggtgggggcagggaagatgcccccgcccaggaaccttcctaccagagtcccaagtttctggggggttcccaggttagcccaagccgtgctaaagctccagtgaacacatatggacctggctttggacccaacttgcccaatcacaagtcaggttcctatcccaccccttcaccatgccatgaaaattttgtagtgggggcaaatagggcttcacatagggcagcagcaccacctcgacttctgcccccattgcccacttgctatgggcctctcaaagtgggaggcacaaaccccagctgtggtcatcctgaggtgggcaggctaggagggggtcctgccttgtaccctcctcccgaaggacaggtatgtaaccccctggactctcttgatcttgacaacactcagctggactttgtggctattctggatgagccccaggggctgagtcctcctccttcccatgatcagcggggcagctctggacataccccacctccctctgggccccccaacatggctgtgggcaacatgagtgtcttactgagatccctacctggggaaacagaattcctcaactctagtgcctaaagagtagggaatctcatccatcacagatcgcatttcctaaggggtttctatccttccagaaaaattgggggagctgcagtcccatgcacaagatgccccagggatgggaggtatgggctgggggctatgtatagtctgtatacgttttgaggagaaatttgataatgacactgtttcctgataataaaggaactgcatcagaaaaaaaaaaaaaaaaaa

SEQ ID NO: 34 - protein, 978 aa, Homo sapiens
  GLI family zinc finger 1 (GLI1) isoform 2
MSPSLGFPAQMNHQKGPSPSFGVQPCGPHDSARGGMIPHPQSRGPFPTCQLKSELDMLVGKCREEPLEGDMSSPNSTGIQDPLLGMLDGREDLEREEKREPESVYETDCRWDGCSQEFDSQEQLVHHINSEHIHGERKEFVCHWGGCSRELRPFKAQYMLVVHMRRHTGEKPHKCTFEGCRKSYSRLENLKTHLRSHTGEKPYMCEHEGCSKAFSNASDRAKHQNRTHSNEKPYVCKLPGCTKRYTDPSSLRKHVKTVHGPDAHVTKRHRGDGPLPRAPSISTVEPKREREGGPIREESRLTVPEGAMKPQPSPGAQSSCSSDHSPAGSAANTDSGVEMTGNAGGSTEDLSSLDEGPCIAGTGLSTLRRLENLRLDQLHQLRPIGTRGLKLPSLSHTGTTVSRRVGPPVSLERRSSSSSSISSAYTVSRRSSLASPFPPGSPPENGASSLPGLMPAQHYLLRARYASARGGGTSPTAASSLDRIGGLPMPPWRSRAEYPGYNPNAGVTRRASDPAQAADRPAPARVQRFKSLGCVHTPPTVAGGGQNFDPYLPTSVYSPQPPSITENAAMDARGLQEEPEVGTSMVGSGLNPYMDFPPTDTLGYGGPEGAAAEPYGARGPGSLPLGPGPPTNYGPNPCPQQASYPDPTQETWGEFPSHSGLYPGPKALGGTYSQCPRLEHYGQVQVKPEQGCPVGSDSTGLAPCLNAHPSEGPPHPQPLFSHYPQPSPPQYLQSGPYTQPPPDYLPSEPRPCLDFDSPTHSTGQLKAQLVCNYVQSQQELLWEGGGREDAPAQEPSYQSPKFLGGSQVSPSRAKAPVNTYGPGFGPNLPNHKSGSYPTPSPCHENFVVGANRASHRAAAPPRLLPPLPTCYGPLKVGGTNPSCGHPEVGRLGGGPALYPPPEGQVCNPLDSLDLDNTQLDFVAILDEPQGLSPPPSHDQRGSSGHTPPPSGPPNMAVGNMSVLLRSLPGETEFLNSSA

SEQ ID NO: 35 - DNA, 3483 bp, Homo sapiens
  GLI family zinc finger 1 (GLI1), transcript variant 3 cDNA
cccagactccagccctggaccgcgcatcccgagcccagcgcccagacagagtgtccccacaccctcctctgagacgccatgttcaactcgatgaccccaccaccaatcagtagctatggcgagccctgctgtctccggcccctccccagtcagggggcccccagtgtggggacagaagtcaagttgaccaagaagcgggcactgtccatctcacctctgtcggatgccagcctggacctgcagacggttatccgcacctcacccagctccctcgtagctttcatcaactcgcgatgcacatctccaggaggctcctacggtcatctctccattggcaccatgagcccatctctgggattcccagcccagatgaatcaccaaaaagggccctcgccttcctttggggtccagccttgtggtccccatgactctgcccggggtgggatgatcccacatcctcagtcccggggacccttcccaacttgccagctgaagtctgagctggacatgctggttggcaagtgccgggaggaacccttggaaggtgatatgtccagccccaactccacaggcatacaggatcccctgttggggatgctggatgggcgggaggacctcgagagagaggagaagcgtgagcctgaatctgtgtatgaaactgactgccgttgggatggctgcagccaggaatttgactcccaagagcagctggtgcaccacatcaacagcgagcacatccacggggagcggaaggagttcgtgtgccactgggggggctgctccagggagctgaggcccttcaaagcccagtacatgctggtggttcacatgcgcagacacactggcgagaagccacacaagtgcacgtttgaagggtgccggaagtcatactcacgcctcgaaaacctgaagacgcacctgcggtcacacacgggtgagaagccatacatgtgtgagcacgagggctgcagtaaagccttcagcaatgccagtgaccgagccaagcaccagaatcggacccattccaatgagaagccgtatgtatgtaagctccctggctgcaccaaacgctatacagatcctagctcgctgcgaaaacatgtcaagacagtgcatggtcctgacgcccatgtgaccaaacggcaccgtggggatggccccctgcctcgggcaccatccatttctacagtggagcccaagagggagcgggaaggaggtcccatcagggaggaaagcagactgactgtgccagagggtgccatgaagccacagccaagccctggggcccagtcatcctgcagcagtgaccactccccggcagggagtgcagccaatacagacagtggtgtggaaatgactggcaatgcagggggcagcactgaagacctctccagcttggacgagggaccttgcattgctggcactggtctgtccactcttcgccgccttgagaacctcaggctggaccagctacatcaactccggccaatagggacccggggtctcaaactgcccagcttgtcccacaccggtaccactgtgtcccgccgcgtgggccccccagtctctcttgaacgccgcagcagcagctccagcagcatcagctctgcctatactgtcagccgccgctcctccctggcctctcctttcccccctggctccccaccagagaatggagcatcctccctgcctggccttatgcctgcccagcactacctgcttcgggcaagatatgcttcagccagagggggtggtacttcgcccactgcagcatccagcctggatcggataggtggtcttcccatgcctccttggagaagccgagccgagtatccaggatacaaccccaatgcaggggtcacccggagggccagtgacccagcccaggctgctgaccgtcctgctccagctagagtccagaggttcaagagcctgggctgtgtccataccccacccactgtggcagggggaggacagaactttgatccttacctcccaacctctgtctactcaccacagccccccagcatcactgagaatgctgccatggatgctagagggctacaggaagagccagaagttgggacctccatggtgggcagtggtctgaacccctatatggacttcccacctactgatactctgggatatgggggacctgaaggggcagcagctgagccttatggagcgaggggtccaggctctctgcctcttgggcctggtccacccaccaactatggccccaacccctgtccccagcaggcctcatatcctgaccccacccaagaaacatggggtgagttcccttcccactctgggctgtacccaggccccaaggctctaggtggaacctacagccagtgtcctcgacttgaacattatggacaagtgcaagtcaagccagaacaggggtgcccagtggggtctgactccacaggactggcaccctgcctcaatgcccaccccagtgaggggcccccacatccacagcctctcttttcccattacccccagccctctcctccccaatatctccagtcaggcccctatacccagccaccccctgattatcttccttcagaacccaggccttgcctggactttgattcccccacccattccacagggcagctcaaggctcagcttgtgtgtaattatgttcaatctcaacaggagctactgtgggagggtgggggcagggaagatgcccccgcccaggaaccttcctaccagagtcccaagtttctggggggttcccaggttagcccaagccgtgctaaagctccagtgaacacatatggacctggctttggacccaacttgcccaatcacaagtcaggttcctatcccaccccttcaccatgccatgaaaattttgtagtgggggcaaatagggcttcacatagggcagcagcaccacctcgacttctgcccccattgcccacttgctatgggcctctcaaagtgggaggcacaaaccccagctgtggtcatcctgaggtgggcaggctaggagggggtcctgccttgtaccctcctcccgaaggacaggtatgtaaccccctggactctcttgatcttgacaacactcagctggactttgtggctattctggatgagccccaggggctgagtcctcctccttcccatgatcagcggggcagctctggacataccccacctccctctgggccccccaacatggctgtgggcaacatgagtgtcttactgagatccctacctggggaaacagaattcctcaactctagtgcctaaagagtagggaatctcatccatcacagatcgcatttcctaaggggtttctatccttccagaaaaattgggggagctgcagtcccatgcacaagatgccccagggatgggaggtatgggctgggggctatgtatagtctgtatacgttttgaggagaaatttgataatgacactgtttcctgataataaaggaactgcatcagaaaaaa

SEQ ID NO: 36 - protein, 1065 aa, Homo sapiens
  GLI family zinc finger 1 (GLI1) isoform 3
MFNSMTPPPISSYGEPCCLRPLPSQGAPSVGTEVKLTKKRALSISPLSDASLDLQTVIRTSPSSLVAFINSRCTSPGGSYGHLSIGTMSPSLGFPAQMNHQKGPSPSFGVQPCGPHDSARGGMIPHPQSRGPFPTCQLKSELDMLVGKCREEPLEGDMSSPNSTGIQDPLLGMLDGREDLEREEKREPESVYETDCRWDGCSQEFDSQEQLVHHINSEHIHGERKEFVCHWGGCSRELRPFKAQYMLVVHMRRHTGEKPHKCTFEGCRKSYSRLENLKTHLRSHTGEKPYMCEHEGCSKAFSNASDRAKHQNRTHSNEKPYVCKLPGCTKRYTDPSSLRKHVKTVHGPDAHVTKRHRGDGPLPRAPSISTVEPKREREGGPIREESRLTVPEGAMKPQPSPGAQSSCSSDHSPAGSAANTDSGVEMTGNAGGSTEDLSSLDEGPCIAGTGLSTLRRLENLRLDQLHQLRPIGTRGLKLPSLSHTGTTVSRRVGPPVSLERRSSSSSSISSAYTVSRRSSLASPFPPGSPPENGASSLPGLMPAQHYLLRARYASARGGGTSPTAASSLDRIGGLPMPPWRSRAEYPGYNPNAGVTRRASDPAQAADRPAPARVQRFKSLGCVHTPPTVAGGGQNFDPYLPTSVYSPQPPSITENAAMDARGLQEEPEVGTSMVGSGLNPYMDFPPTDTLGYGGPEGAAAEPYGARGPGSLPLGPGPPTNYGPNPCPQQASYPDPTQETWGEFPSHSGLYPGPKALGGTYSQCPRLEHYGQVQVKPEQGCPVGSDSTGLAPCLNAHPSEGPPHPQPLFSHYPQPSPPQYLQSGPYTQPPPDYLPSEPRPCLDFDSPTHSTGQLKAQLVCNYVQSQQELLWEGGGREDAPAQEPSYQSPKFLGGSQVSPSRAKAPVNTYGPGFGPNLPNHKSGSYPTPSPCHENFVVGANRASHRAAAPPRLLPPLPTCYGPLKVGGTNPSCGHPEVGRLGGGPALYPPPEGQVCNPLDSLDLDNTQLDFVAILDEPQGLSPPPSHDQRGSSGHTPPPSGPPNMAVGNMSVLLRSLPGETEFLNSSA

SEQ ID NO: 37 - DNA, 3414 bp, Homo sapiens; identical to SEQ ID NO: 33
  GLI family zinc finger 1 (GLI1), transcript variant 2 cDNA

SEQ ID NO: 38 - protein, 978 aa, Homo sapiens; identical to SEQ ID NO: 34
  GLI family zinc finger 1 (GLI1) isoform 2

SEQ ID NO: 39 - DNA, 51 bp, Artificial Sequence
  synthetic TaqMan assay probe sequence specific for GLI1 SNP rs2228224 allele
taccagagtcccaagtttctgggggrttcccaggttagcccaagccgtgct

SEQ ID NO: 40 - DNA, 51 bp, Artificial Sequence
  synthetic TaqMan assay probe sequence specific for MDR1 SNP rs2032582 allele
tatttagtttgactcaccttcccagmaccttctagttctttcttatctttc

SEQ ID NO: 41 - DNA, 51 bp, Artificial Sequence
  synthetic TaqMan assay probe sequence specific for MDR1 SNP rs2032582 allele
tatttagtttgactcaccttcccagyaccttctagttctttcttatctttc

SEQ ID NO: 42 - DNA, 51 bp, Artificial Sequence
  synthetic TaqMan assay probe sequence specific for ATG16L1 SNP rs2241880 allele
cccagtcccccaggacaatgtggatrctcatcctggttctggtaaagaagt

SEQ ID NO: 43 - DNA, 51 bp, Artificial Sequence
  synthetic TaqMan assay probe sequence specific for GLI1 SNP rs2228224 allele
taccagagtcccaagtttctgggggattcccaggttagcccaagccgtgct

SEQ ID NO: 44 - DNA, 51 bp, Artificial Sequence
  synthetic TaqMan assay probe sequence specific for GLI1 SNP rs2228224 allele
taccagagtcccaagtttctggggggttcccaggttagcccaagccgtgct

SEQ ID NO: 45 - DNA, 51 bp, Artificial Sequence
  synthetic TaqMan assay probe sequence specific for MDR1 SNP rs2032582 allele
tatttagtttgactcaccttcccagcaccttctagttctttcttatctttc

SEQ ID NO: 46 - DNA, 51 bp, Artificial Sequence
  synthetic TaqMan assay probe sequence specific for MDR1 SNP rs2032582 allele
tatttagtttgactcaccttcccagaaccttctagttctttcttatctttc

SEQ ID NO: 47 - DNA, 51 bp, Artificial Sequence
  synthetic TaqMan assay probe sequence specific for MDR1 SNP rs2032582 allele
tatttagtttgactcaccttcccagtaccttctagttctttcttatctttc

SEQ ID NO: 48 - DNA, 51 bp, Artificial Sequence
  synthetic TaqMan assay probe sequence specific for ATG16L1 SNP rs2241880 allele
cccagtcccccaggacaatgtggatactcatcctggttctggtaaagaagt

SEQ ID NO: 49 - DNA, 51 bp, Artificial Sequence
  synthetic TaqMan assay probe sequence specific for ATG16L1 SNP rs2241880 allele
cccagtcccccaggacaatgtggatgctcatcctggttctggtaaagaagt